SEQUENCE LISTING (1 sequence)

SEQ ID NO: 1 - DNA, 77 bp, synthetic construct
  source 1..77: mol_type = other DNA
tttttggcaggaagacaaacacgatgggggggtatgatttgatgtggttgttgcatgatcgtggtctgtggtgctgt

(19) United States
(12) Patent Application Publication
(10) Pub. No.: US 2026/0072021 A1
(43) Pub. Date: Mar. 12, 2026

(54) SYSTEM, METHOD, AND APPARATUS FOR MICRONEEDLE ARRAY-BASED IMMUNOSENSORS FOR MULTIPLEX DETECTION OF BIOMARKERS

(71) Applicant: The General Hospital Corporation, Boston, MA (US)

(72) Inventors: Meixiong Wu, Boston, MA (US); Zuan-Tao Lin, Boston, MA (US)

(21) Appl. No.: 18/864,139

(22) PCT Filed: May 8, 2023
(86) PCT No.: PCT/US2023/066737

§ 371 Date: Nov. 8, 2024

Related U.S. Application Data
(60) Provisional application No. 63/339,487, filed on May 8, 2022.

(51) Int. Cl. G01N 33/543; G01N 21/64; G01N 33/58

(52) U.S. Cl. CPC G01N 33/54353; G01N 21/6456; G01N 33/54366; G01N 33/581; G01N 2333/908

(57) ABSTRACT

A system for identifying a plurality of biomarkers in a sample, including: a substrate including a plurality of microneedles projecting therefrom, each of the plurality of microneedles including a plurality of biomarker recognition molecules attached thereto, and the plurality of microneedles including a first microneedle and a second microneedle, the first microneedle including a first plurality of biomarker recognition molecules configured to recognize a first biomarker, and the second microneedle including a second plurality of biomarker recognition molecules configured to recognize a second biomarker different from the first biomarker.

### CROSS-REFERENCE TO RELATED APPLICATIONS

The present application is the national stage entry of PCT/US2023/066737, filed on May 8, 2023, which is based on and claims priority from U.S. Patent Application Ser. No. 63/339,487, filed on May 8, 2022, the entire disclosure of which is incorporated herein by reference.

### STATEMENT REGARDING FEDERALLY SPONSORED RESEARCH

This invention was made with government support under grant number FA9550-17-1-0277 awarded by the Air Force Office of Scientific Research and grant number HU0001-17-2-0009 awarded by Military Medicine Technology Transformation Collaborative via the Uniformed Services University of the Health Sciences (USU). The government has certain rights in the invention.

### SEQUENCE LISTING

This application includes a sequence listing in XML format titled “2023-05-08_125141_04326_WIPO_Sequence_listing.xml”, which is 1,945 bytes in size and was created on May 8, 2023. The sequence listing is electronically submitted with this application via Patent Center and is incorporated herein by reference in its entirety.

### BACKGROUND

In the past two decades, we have witnessed an ever-growing demand for point-of-care (PoC) or onsite diagnostic tools that can measure a wide range of analytes. This trend is driven by a rapid rising in digital health eCare that confronts with a relatively long turnaround time through clinical laboratories equipped with a full spectrum of cutting-edge facilities, a routine clinical practice for a half century. Indisputably, the faster the physicians receive test results, the sooner they can make a critical care decision giving rise to much better outcomes, especially for infection diseases, traumatic injuries, and cardiovascular disorders. Moreover, monitoring of both disease progress and treatment effectiveness remotely have become common medical practices in cancer treatments and care of chronic diseases and elderly. The PoC device under this invention acting as a remote extension to clinical laboratories would benefit millions of these patients in both urgent and long-term cares in hospitals and at home around the world.

Disease diagnosis, prognosis, and monitoring, and pathogenic process determination usually involve complicated biological activities and could not be readily determined by a single biomarker. To sufficiently diagnose or assess pathogenic process, several or a panel of specific biomarkers must be taken into consideration. Clinical studies have clearly demonstrated that a panel of biomarkers can improve the precise of disease diagnosis considerably for many diseases. Traditionally, detection of a panel of biomarkers relies on sophisticated and expensive instruments in laboratory settings and is conducted by skilled medical staff, especially for some biomarkers at a low abundance in blood or in some body fluids due to a low detection limit of the instruments. In most of cases, it takes days from sample collection to reports of the test results, which is not acceptable for some acute diseases like infection and traumatic injuries. In the past two decades, home-based technologies for measuring blood glucose in management of diabetes, urine chorionic gonadotropin (HCG) for pregnancy, and recently COVID-19 viral Spike protein demonstrate a great success and benefit millions of patients. However, these tests each can measure only one biomarker and the technologies are not extending to other diseases or healthcare needs. A large group of scientists have devoted enormous efforts to replicate the success of these home-based tests for measuring multi-biomarkers in the past two decades. But, as of today, there are no commercial point-of-care diagnostic tools that can simultaneously measure a panel of biomarkers remotely without a laboratory. While the golden standard enzyme-linked immunoassay (ELISA) is a routine assay for multi-biomarker detection in the clinics, the assay depends on sophisticated instruments and trained technicians in fully equipped laboratories. Other technologies capable of detecting multi-biomarkers, such as protein microarray, rely on expensive equipment and extensively trained staffs and hardly become a lab-free routine.

Currently, there are no assays that can detect a panel of biomarkers as reliable, accurate, and sensitive as traditional ELISA without the need for a laboratory although sensors capable of measuring a single analyte like glucose, chorionic gonadotropin (HCG) are widely used at the home. ELISA relies on expensive instruments and highly skilled technicians in the laboratory setting.

### SUMMARY OF THE INVENTION

Accordingly, new systems, methods, and apparatus for identifying a plurality of biomarkers in a sample are desirable.

In one embodiment, a system for identifying a plurality of biomarkers in a sample, including: a substrate including a plurality of microneedles projecting therefrom, each of the plurality of microneedles including a plurality of biomarker recognition molecules attached thereto, and the plurality of microneedles including a first microneedle and a second microneedle, the first microneedle including a first plurality of biomarker recognition molecules configured to recognize a first biomarker, and the second microneedle including a second plurality of biomarker recognition molecules configured to recognize a second biomarker different from the first biomarker.

In another embodiment, a method for identifying a plurality of biomarkers in a sample, including: providing a substrate including a plurality of microneedles projecting therefrom, each of the plurality of microneedles including a plurality of biomarker recognition molecules attached thereto, and the plurality of microneedles including a first microneedle and a second microneedle, the first microneedle including a first plurality of biomarker recognition molecules configured to recognize a first biomarker, and the second microneedle including a second plurality of biomarker recognition molecules configured to recognize a second biomarker different from the first biomarker; contacting the plurality of microneedles with the sample such that at least one biomarker of the plurality of biomarkers in the sample is coupled to at least one biomarker recognition molecule of the plurality of biomarker recognition molecules; and processing the plurality of microneedles to identify the at least one biomarker of the plurality of biomarkers in the sample.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIG. 1 shows design, fabrication, and working principle of MNA-immunosensors according to certain constructions. Panel a. An optical transparent MNA is prepared by casting PMMA into a female PDMS MNNA mold, follow by modification with PEI. PAMAM dendrimer is covalently attached to the surface of each MN via PEI and further enrich amine groups and increase the conjugated rate of capture antibody. Panel b. MNA-aptasensor, which is modified with specific aptamers, can detect target molecules directly by using florescent signal. Panel c. Capture antibody is conjugated with amine groups of the dendrimer on the MNA and subjected to a micro-ELISA including blockade on non-specific binding, biomarker incubation, 2^{nd} antibody addition, and substrate reactions. Panel d. Direct imaging of each MN in the array. Panel e. NIA-linked gel assay enables transfer of colorimetric signal on MNA to a gel, converting the three-dimensional (3D) signal from each of the MNs to a 2D signal.

FIG. 2 shows two alternative methods to conjugate capture antibodies or other biomarker recognition molecules on the surface of individual MNs in the MNA in a one-antibody-on-one microneedle fashion. Panel a. A piece of double-side tape is punched by laser mirroring the MNA pattern to cover the MNA base. Each hole in tape and the surface of MNA forms a micro-container, allowing conjugation of a capture antibody occurring around the individual MN. Panel b. A PMDS female MNA mold is fabricated with an array of micro-columns in place of microneedle geometric array, each of which can hold a specific antibody reaction solution for individual MNs in the array.

FIG. 3 shows functionality of MNA color assays on a gel. Panel a. Image of a stained gel in response to various human CRP from 0.925 to 75 pg/mL in PBS or 75 pg/mL BSA and PBS controls, which is enlarged from b (low panel). Scale bar: 200 μm. Panel b. Image of a stained gel corresponding to 75, 50, 37.5, 18.5, 9.25, 4.75, 2.25, and 0.925 pg/mL of human CRP in duplicate (left to right). Scale bar: 1 mm. Panel c. 3D images of stained spots on gel corresponding to the spots displayed in (Panel a). Scale bar: 200 μm. Panel d. Kinetics of CRP, IFNα-2a and PCT MNA colorimetric assays on a single gel in response to various concentrations of human CPR, mouse CRP, IFNα-2a and PCT or BSA in PBS. Panel e. Calibration curves of CRP, IFNα-2a and PCT MNA colorimetric assay are obtained from a single gel.

FIG. 4 shows calibration curves of MNA colorimetric assays on a gel based on MNA with (right) and without (left) dendrimer modification. About an 20× increase in sensitivity is attained in the presence compared to the absence of dendrimer.

FIG. 5 shows functionality of colorimetric assays on the surface of MNs. Panel a. Images of stained MNs in response to human IGFBP2 at concentrations from 18 to 480 pg/mL in PBS, 200 pg/mL BSA and PBS controls. Panel b. Kinetics of four colorimetric assays on a single MNA corresponding to various concentrations of human CPR, mouse CRP, IGFBP2, TNFRII, VCAM-1, or BSA in PBS. Panel c. Calibration curves of CRP, IGFBP2, TNFRII and VCAM-1 colorimetric assays on surface of MNs in a single MNA.

FIG. 6 shows calibration curves of CRP colorimetric assays on an MNA with and without dendrimer modification. About a 55× increase in sensitivity is attained in the presence compared to the absence of dendrimer.

FIG. 7 shows detection of various biomarkers using MNAs. Panel a. IGFBP2, TNFRII, and VCAM-1 levels in serum samples from lupus patients and healthy controls were measured by the colorimetric assays on surface of MNs of MNA; Panel b. The IGFBP2, TNFRII and VCAM-1 levels in the same serum samples were determined by commercial ELISA kits. Panel c. The paired correlations are analyzed to confirm similar sensitivity, accuracy, and reproductivity of the two assays.

FIG. 8 shows detection of anti-double stranded DNA (anti-dsDNA) antibodies using MNAs. Panel a. Anti-dsDNA antibody levels in serum samples collected from lupus patients and healthy controls were measured by the colorimetric assays on surface of MNs in a single MNA; Panel b. Anti-dsDNA antibody levels in the same serum samples were determined by commercial ELISA kits showing similarity of the two assays.

FIG. 9 shows that use of an optically transparent MNA enhances light transmittance. Panel A. Schematic of light transmittance measurement: Subpanel a. Laser passes through a micro-hole array; Subpanel b. Laser passes through an MNA base with corresponding micro-holes; Subpanel c. Laser illuminates via inverted transparent MNA; and Subpanel d. Laser penetrates via a transparent MNA with enhancing transmittance (deep green). The green colors simulate light transmittance efficiency. Panel B. Percentages of transmittance spectra over the visible and near infrared laser. Note. The higher light transmittance is obtained via an optically transparent MNA (Subpanel d or red in Panel B).

FIG. 10 shows schematic light transmission efficiency via a transparent MNA in the presence of skin tissue. Panel a. Initial laser intensity for laser irradiation; Panel b. laser irradiation via a micro-hole array; Panels c, d, e. Laser irradiation via a transparent MNA inserted through the micro-hole array in the absence of skin tissue (Panel c) or in the presence of 0.13-mm-thick ear skin tissue (Panel d) or 0.45-mm-thick skin tissue (Panel e). The numbers in Panels b-e were the laser intensity transmitted via the indicated barrier when 1.99 mW green laser (Panel a) was applied onto the barrier.

FIG. 11 shows a construction of an MNA-aptasensor. (Panel A) A “Signal-On” aptasensor for cocaine (COC) detection is formed on individual MNs in an MNA. AF594-labeled FDNA is the fluorescence “OFF” state due to FRET between AF594 and BHQ-2. Addition of COC or BZE results in the release of BHQ-2-labeled QDNA and the formation of cocaine-aptamer complex, upon which AF594 is activated to the fluorescence “ON” state. (Panel B) Fluorescence spectra of MN-based aptasensor in the presence of various concentrations of COC (0-5 mM) dissolved in 1×PBS buffer (pH=7.4).

FIG. 12 Panel A shows a schematic of dendrimer-based signal amplification. Panel B shows fluorescence spectra of MNA-based aptasensor in the presence of various concentrations of COC (0-1 mM) dissolved in 1×PBS buffer (pH=7.4).

FIG. 13 Panel a shows an optical fiber for delivering excitation light and sensitively collecting emission light from or into a single microneedle in an MNA. An optical fiber of 500 μm in diameter was engineered to include 6 emission light collection fibers each at 125 m in diameter, surrounding the single excitation light fiber at the center [a longitudinal section (Panel a) and cross-section (Panel c)]. Panel b. Photos of the fiber outlet (left) and the fiber (right).

FIG. 14 shows real-time detection of COC and BZE in mice. Panel A. Time-dependent blood concentrations of COC and BZE in mice receiving 1.5 mg/Kg cocaine per mouse via IP injection. Panel B. Experimental setup of in vivo detection of circulating COC/BZE using MN-based aptasensor on a B6 black mouse. Panel C. Time-dependent fluorescence intensity of an individual MN that pierces into the mouse skin.

FIG. 15 shows a miniDia device and its accessories according to various constructions. There are three versions mainly in plasma separation as depicted in Panels a, b, and c. Panel a. Plasma separation is accomplished by a membrane placed in the sample chamber of the regents-prefilled microfluidic array. Panel b. The miniDia equipped with a disk holding a capillary tube. A detachable capillary blood collection tube and disk-centrifuge is integrated into the miniDia device. Panel c. The miniDia equipped with a disk that contains a capillary blood collection tube for blood loading. A capillary blood collection tube and disk centrifuge are needed.

FIG. 16 shows systems and procedures for self-sample collection and processing. Self-blood sample collection starts with the lancet to prick the finger, collect the blood sample, and transfer it to the sample chamber of the reagents-prefilled microfluidic array. The blood sample collection device has a capillary channer, buffer, and a buffer barrel in the syringe-life cylinder (Panel a). The sample is drawn into the capillary channel and mixed with the buffer, followed by pressing it into the sample chamber of the microfluidic array (Panel b). The blood cells are removed by the plasma separation membrane. Continuously pressing the syringe plunger allows the buffer to go through the membrane and also dilute the sample.

FIG. 17 shows design and structure of disk-centrifuges. A disk-centrifuge may be embedded in the phone case. The disk-centrifuge includes a mini motor, mini motor chamber, mini motor chamber cover, phones, desk, and disk cover. A disk-centrifuge for capillary tubes (Panel a) or capillary channels (Panel b).

FIG. 18 shows sample collection and preparation with a disk-centrifuge containing a capillary tube. To balance the centrifuge, there are two capillary tube holders on the opposing sides of the disk. A detachable capillary tube is designed to collect a blood sample. Two blockers are utilized to seal the tube, and a capillary tube with a similar weight (not shown) is attached to the kit for balance. After centrifugation, the tube is taken out and separated by pushing the connector. The tube with serum is inserted to the sample chamber of the microfluidic array.

FIG. 19 shows sample collection and preparation for a disk centrifuge containing capillary channels. The transparent disk contains two capillary channels. The blood sample collected by a capillary blood collection tube is unloaded into the capillary channel through the inlet. The same volume of blood sample or water is injected into the other capillary channel for balance. Once centrifugation is completed, plasma is collected and transferred to the microfluidic array.

FIG. 20 shows a microfluidic array pre-filled with reagent. Panel a. Reagent pre-filled microfluidic array may be 3D printed using transparent material for monitoring the fluid influx into the MNA housing chamber visually. The sample may be loaded into the sample chamber via a connector. A plasma separation membrane may be embedded in the bottom of the connector (left) for blood samples, but not (right) for plasma samples collected from a disk-centrifuge. Reagents used in assays are injected via reagent inlet holes. A vent hole is designed to remove the air within the microfluid channel during the reagent and sample loading steps. The outlet close to the sample chamber is connected to the immunoassay station and the other outlet is installed with a one-way valve. Panel b. One-way valve and pre-filled microfluidic array. Reagents used in assay such as washing buffer, detection antibodies, and substrate are pre-filled sequentially in the corresponding segments.

FIG. 21 shows detachable components for miniDia. Panel a. Four accessories for inserting MNA or gel into the MNA housing chamber or covering the gel. Panel b. The structure and working principle of the detachable components. A gel cover, or MNA can be placed onto the handle or removed from the handle.

FIG. 22 shows a vertical cross-sectional view of a miniDia apparatus. The MiniDia may include an immunoassay station (blue outline) and imaging section (red outline) (Panel a). Panel b. The immunoassay station encompasses a one-way valve to create vacuum in the vacuum chamber that can draw the reagents pre-filled in the microfluidic array into the MNA housing chamber where an immunoassay is conducted. The immunoassay station must be air-tight sealed. Panel c. The imaging section contains (*) gel station for MNA-inked gel assay only, and phone, App, micro-lens, and LED light together to capture and analyze the images on the MNA or a gel.

FIG. 23 shows the working principle of the immunoassay station. Two one-way valves are used to draw an influx of reagents sequentially from the microfluidic array to the MNA housing chamber. One is connected to a vacuum chamber, and the other to the reagent-pre-filled microfluidic array. When the button of vacuum chamber is pressed, the air in the vacuum chamber is let out through the one-way valve in the MNA housing (pink arrows, left) and, as the spring-loaded plunger retracts, negative pressure is generated in the vacuum chamber and reagent-pre-filled microfluidic array. The negative pressure pulls the reagents to the MNA housing chamber from the microfluidic arrays, and the solutions are finally collected in the reagent waste container (pink arrows, right). The solutions from the pre-filled capillary array are delivered to and move across the MNA in the order in which they are filled in the capillary array: first the diluted sample, followed by one or more boluses of wash solution, then the detection antibody mixture followed by the substrate, and finally one or more wash solutions.

FIG. 23A shows an alternative design and working principle for the immunoassay station. The immunoassay station is outlined in blue dash lines. The imaging station is outlined in red dash lines (3) and placed on top of the MNA housing chamber (MNA). The immunoassay is operated by a stepper motor (1) to draw reagents prefilled in the microfluidic array (2) sequentially into the MNA housing chamber (pink arrows).

FIG. 24 shows a vertical cross-sectional view of the imaging section. Imaging acquisition of the immunoassay station for (Panel a) MNA-inked gel assay can be accomplished with a single micro lens, while (Panel b) a magnifier with a micro lens set is required for direct imaging of each MNA. Other components are similar for the two modes (gel-based vs. direct imaging) including ring LED light, phone holder, and smartphone.

FIG. 25 shows a vertical cross view of the mechanical stage. The mechanical stage is used to hold and lift MNA for precisely imaging the MNA or a gel.

FIG. 26 shows an imaging section of the device. The MNA and the gel are inserted into the MNA housing chamber and a gel holder respectively, using the detachable handle (Panel a), with a gel cover placed between the two (Panel b). After the immunoassay is completed, the gel cover is removed and the MNA is lifted directly into the gel. Finally, the LED light is turned on to illuminate the gel and the cell phone camera can capture the image via a micro lens (Panel c).

FIG. 27 shows direct imaging of individual microneedles in the MNA. After the immunoassay is completed, the MNA is lifted, the LED light is turned on to illuminate the MNA, and images of each microneedle in the MNA are acquired one-by-one.

FIG. 28 shows the workflow (flow chart, left) and screenshots (right) of the smartphone app.

FIG. 29 shows the workflow of a construction of the miniDia. Panel A. Preparation is completed by registering and scanning QR, insertion of MNA and a gel, and connection of the microfluidic array to the miniDia. Panel B. The samples can detect a drop of blood or any other body fluid using a sample volume of as little as 50-100 l. The sample is added to the sample chamber for the microfluidic array. Panel C. The immunoassay is performed by repeat pressing of the button following the instruction of a specific test. Panel D. Photos or images of the MNA are taken by smartphone and analyzed by App for result reports.

FIG. 30 shows: (Panel a) a schematic of the portable optical prototype: 1) CCD camera connected with smartphone; 2) long-pass filter; 3) zoom lens; 4) objective lens; 5) cube beam splitter; 6) multi-beam source system; 7) MNA-aptasensor that is wearable and directs light into the upper dermis. (Panel b) Light source system. A micro lens array is placed in the front of a laser source. The micro lens array is covered with a mask to provide multi-beams of the lasers in order to direct a separate beam into each of the MNs of the MNA. In some cases, a convex microlens array is mounted directly on the top of MNA in a microlens-on-one microneedle fashion to increase light penetration into the epidermis and dermis, especially for people with dark skin color. (Panel c) A representative fluorescent photograph of the four microneedles, excited under 532 nM laser, was captured by a smartphone. The four microneedles were covalently modified on their surface by a fluorophore Cy3.5-linked cocaine-specific aptamer at concentrations of 1, 10, 100, or 1,000 nM, respectively.

FIG. 31 shows steps of preparing an MNA gel assay. Panel a. Schematic illustration of surface modification of MNA. The MNA was prepared by casting PMMA into a PDMS-MNA mold. After plasma treatment, the surface of the MNA was coated with PEI, followed by conjugation with PAMAM dendrimer. PAMAM dendrimer is employed to further enrich amine groups to increase the efficiency of capture antibody conjugation, thereby enhancing the sensitivity of assay. Panel b. The scanning electron microscope (SEM) image of MNA. Panel c. Method of immobilization of multiplex capture elements on a single MNA in one capture element on one MN fashion. The MNA base was covered by a double-side tape that was pre-treated with a laser to punch an array of tiny holes precisely aligning with the microneedles in the MNA, such that each hole formed a micro-container, allowing the capture elements reaction mixture to surround the individual microneedle to immobilize specific capture antibody via EDC/NHS coupling reaction. Panels d and e. Process and working principle of MNA gel assay. Upon the addition of a serum or other type of sample on the MNA, the specific capture antibodies on the surface of microneedles capture the target molecules. After addition of biotin-detection antibody mixtures and streptavidin-HRP, an immunoassay sandwich formation is formed on the surfaces of each of the microneedles. Since gel is immersed in and saturated with colorimetric substrate, a blue precipitate formed by the substrate in presence of HRP of the immunoassay sandwich formation on microneedles and accumulates surrounding the locations where microneedles are inserted into the gel. Therefore, MNA gel assay allows for transfer of colorimetric signal on MNA to a gel, making the three dimensions (3D) signal to 2D signal, which can simplify the imaging method.

FIG. 32 shows functionality of the MNA gel assay for detection of high abundance protein and low abundance protein. CRP and TNFRII were selected as a representative of high abundance protein (>100 ng/mL in serum) and low abundance protein (<100 ng/mL in serum), respectively to examine the functionality of MNA gel assay. Panel a. Top left: Image of stained gel in response to various human CRP in a range from 80 to 1,000 pg/mL in PBS and 500 pg/mL BSA. Scale bar: 200 μm. Bottom, left: Image of stained gel in response to 1,000, 750, 500, 250, and 80 pg/mL of human CRP (left to right). Scale bar: 1 mm. Bottom, right: 3D image of stained spots on gel corresponding to the spots which are displayed in stained gel image Scale bar: 200 μm. Panels b-c. Kinetics of MNA gel assay response to various concentrations of human CPR, mouse CRP, or BSA in PBS, and the calibration curve of MNA-inked gel assay for CRP detection. Panel d. Standard curve of commercial CRP ELISA kit (R&D). Panel e. Comparison of signal intensity ratio of MNA for CRP detection where CRP captures antibody immobilized on surface of MNA with different treatments (n=3 independent tests), compared with the original MNA. Panel f Standard curve of MNA gel assay using MNA without dendrimer decoration. Panel g. Comparison of kinetics of MNA gel assay based on MNA with and without dendrimer decoration response to various concentrations of CPR. Panels h-j. The performance of MNA decorated with dendrimers. Calibration curves of MNA gel assays using MNA with decoration of dendrimer G0, G4, and G6 (left to right) were used to evaluate performance of MNA decorated with different dendrimers. The LoD value are 18.30 pg/mL, 1.41 pg/mL, 0.89 pg/mL, and 1.36 pg/mL for G0, G4 and G6. Panels k-l. LoD improvement obtained by MNA with decoration of dendrimer G0, G4, G5 and G6 in comparison with MNA without dendrimer decoration (Panel k) and with commercial CRP ELISA (Panel 1). Panels m-n. Image of stained MNA without microneedles in response to various human CRP in a range from 80 to 1,000 pg/mL in PBS, and its calibration curve. Panel o. Comparison of signal intensity of MNA gel assay and stained MNA without microneedles for CRP detection. Panels p-q. Image of stained gel based on MNA without using the method of immobilization of capture elements in one capture element on one MN fashion in response to various human CRP in a range from 80 to 1,000 pg/mL in PBS, and its calibration curve. It is notable that the background staining is evident due to the lack of protection of tape. Panels r-s. Image of stained MNA in response to various human CRP in a range from 80 to 1,000 pg/mL in PBS, and its calibration curve. Panel t. LoD improvement obtained by MNA gel assay in comparison to direct MNA staining, MNA modified with capture antibody without use of tape and MNA without microneedles. Panels u-v. Kinetics of MNA gel assay response to various concentrations of human TNFRII or BSA in PBS, and its calibration curve. Panel w. Standard curve of commercial TNFRII ELISA kit (R&D). Panel x. The LoD ratio of ELISA and MNA gel assay for detection of CRP and TNFRII. (Data show mean±s.d. a.u., arbitrary units.)

FIG. 33 shows functionality and validation of the MNA gel assay using ELISA. Panels a-b. Kinetics of MNA gel assay response to various concentrations of human IGFBP2, VCAM-1 or BSA in PBS, and the calibration curve of MNA gel assay for IGFBP2 and VCAM-1 detection. Panel c. The LoD ratio of ELISA and MNA gel assay for detection of IGFBP2 and VCAM-1. Panel d. LoD improvement obtained by MNA with dendrimer decoration in comparison with MNA without dendrimer decoration. Panel e. The CRP, IGFBP2, TNFRII, VCAM-1 and anti-dsDNA antibody levels in serum samples from lupus patients (n=42) and healthy controls (n=34) were measured by MNA-inked gel assays and corresponding ELISA kits; The paired correlation test was carried out for the MNA-inked gel assay versus the ELISA results. (Data show mean±s.d. NS, not significant. ** P=0.0016, ***=0.0008, **** P<0.0001 by two-tailed unpaired t-test. a.u., arbitrary units.)

FIG. 34 shows the fabrication and performance of MNA for multiplex biomarker detection and validation by ELISA. Panel a. The method of immobilization of multiplex capture elements on a single MNA in one capture element on one MN fashion. After MNA was covered by a lased pre-treated double-side tape, capture antibodies coupling reaction solutions were added into the targeted micro-container formed by the tape, which located in the biomarkers map on MNA surface (Panel b). For detection of anti-dsDNA antibody, the microneedle was conjugated with mBSA and then incubated with dsDNA. Panel c. Plot exhibiting the cross-reactivity study. We fixed the concentration of detected biomarker at 20 pg/mL while increased the concentration of other biomarkers from 10 to 400 pg/mL to prepare various solutions for this test. Panels d-e. Representative gel images of healthy controls and SLE patients. Panel f. The paired correlation test was carried out for the MNA-inked gel assays versus the ELISA results.

FIG. 35 shows the profiling and statistical analysis of a biomarker panel for SLE diagnosis. Panel a. Schematic illustration of our pipeline to develop a biomarker panel for diagnosis of SLE. Patients with dsDNA antibody) detected by ELISA SLE (n=42) and healthy controls (n=34) were recruited, and blood samples were collected, followed by centrifuge and storage. MNA gel assay and ELISA were carried out for side-by-side measurement of serum levels of selected biomarkers on the same samples. A logistic regression model with five-fold cross-validation was employed to evaluate the diagnostic ability of selected biomarkers, and the results are summarized in Table 4. Panel b. For each group, top: The heat map showing the normalized expression concentration of five biomarkers (CRP, IGFBP2, TNFRII, VCAM-1, and anti- and MNA gel assay. Bottom: According to the diagnostic statistics from Table 4, we develop and define a diagnostic index, ITA, which is calculated by a weighted sum of three biomarkers (IGFBP2, TNFRII, and anti-dsDNA antibody) using the application of logistic regression analysis. The biomarkers panel is able to distinguish SLE patients from healthy controls measured by ELISA and MNA-inked assays and ELISA kits. Panels c.d. Receiver operating characteristic (ROC) analysis was utilized to examine the capability of these three biomarkers to discriminate SLE patients and healthy controls. Both biomarker panel detected by NMA gel assay and ELISA showed excellent discriminative capability in distinguishing SLE patients from healthy control, with AUC values of 0.9881 and 0.9853 respectively. In both cases, all the AUC values of single biomarkers (ELISA group, IGBPF2: 0.8725, TNFR2: 0.9461, anti-dsDNA antibody: 0.8901) (MNA gel assay group, IGBPF2: 0.8662, TNFR2: 0.9531, anti-dsDNA antibody: 0.9104) was smaller than that of the biomarker panel. Panel e, Cut-off values are 1.5240 and −0.9133 for ELISA and MNA gel assay, respectively, which are determined by the highest value of the sum of sensitivity and specificity generated by ROC analysis. The ITA values in SLE patients were significantly higher than those in controls. (**** P<0.0001 by two-tailed unpaired t-test. a.u., arbitrary units.), Panel f. The cut-off value of ITA can differentiate SLE patients from healthy controls.

FIG. 36 shows a schematic of fabrication of an MNA. PDMS and cure solution were poured into a well of well plate and an original male MNA was added. After polymerization, the PDMS-MNA mold was obtained by removal of the original male MNA mold. The PMMA MNA was prepared by casting PMMA solution into the PDMS-MNA mold.

FIG. 37 shows the impact of the concentration of capture antibody and detection antibody. The optimal concentration of capture antibody and detection was identified by determine the signal intensity obtained to measure the lowest concentration of the biomarker in the detection dynamic range of the corresponding ELISA kits.

FIG. 38 shows the impact of staining time. It is found that the signal intensity was increased over time, while it was slightly reduced, which might be attributed to the elevation of background.

FIG. 39 shows the calibration curve of an MNA-inked gel assay without decoration of dendrimer for detection of IGFBP2, TNF-RII, and VCAM-1 (left). Plot showing MNA-inked gel assays, compared with traditional ELISA kits (right).

FIG. 40 shows the calibration curves of ELISA kits for detection of human IGFBP2 and VCAM-1; calibration curves for CRP and TNFRII are shown in FIGS. 32d and 32w, respectively, and thus are not repeated here.

FIG. 41 shows the paired correlation test was conducted for NIA-inked gel assay for single biomarker detection versus MNA-inked gel assay for multi-biomarker detection.

Table 3. Functionality comparison of commercial ELISA kits with MMA-inked gel assay.

Table 4. The comparison of SLE diagnostic statistics of selected biomarker panel and traditional ANA testing.

Table 5. The serum levels of human CRP, IGFBP2, TNF-RII, and VCAM-1, anti-dsDNA antibody measured by MNA-inked gel assay and ELISA kits.

Table 6. Feature comparison of emerging MNA-based sensor with MMA-inked gel assay.

### DETAILED DESCRIPTION

In accordance with some embodiments of the disclosed subject matter, mechanisms (which can include systems, methods, and apparatus) for identifying a plurality of biomarkers in a sample are provided.

The present disclosure relates generally to methods and a small “all-in-one” portable device, named mini-device for immunoassay or miniDia, for multiplex quantification of biomarkers at the home, bedside, and anyplace remotely in a laboratory-free manner, which builds upon earlier work by the present group (see U.S. Pat. No. 10,500,412, the entire disclosure of which is incorporated herein by reference) in related technology.

In one embodiment, a device is provided which includes three portions: (1) sample processing accessories, (2) NIA-immunosensor, and (3) a cellphone-based imaging platform. The sample processing accessory can process any fluid samples collected from humans or animals having a volume as little as 50-100 μl. The fluid samples include but are not limited to, a drop of blood, nasal or throat swabs, urine, stools, tears, and the like.

The immunosensor includes a functionalized microneedle array (MNA) in which the surface of each microneedle is covalently conjugated with a specific antibody (Ab), aptamer, or ligand, collectively called capture elements, in a one-microneedle-one capture element fashion. The resultant MNA can measure a few, dozens, or hundreds of biomarkers in a single array simultaneously. We have successfully measured several biomarkers in a single array in sera of lupus patients and found that the sensitivity, reliability, and accuracy of the MNA are at least 10-times better than the traditional enzyme-linked immunosorbent assay (ELISA), a primary assay that measures biomarkers for diagnosing a variety of diseases in the clinics currently. We have also fabricated an MNA for distinguishing bacterial infection from viral infection or for diagnosing traumatic brain injury (TBI). Different MNAs can be fabricated specifically to detect a set of biomarkers in 2-3 hrs with a drop of blood samples or any samples as small as 50-100 μl prepared from the nasal, throat, oral, and so on. The immunosensor can be processed in the immunoassay station including a reagent-prefilled microfluidic array that can direct the pre-filled reagents to sequentially influx into the MNA housing chamber. The immune assay station functions semi-automatically or automatically in replicating the traditional immune assay on a scale 50-×smaller than ELISA without the need for a laboratory. The fluorescence or colorimetric substrate deposited on each microneedle in the array as a consequence of immunoassay can be acquired and analyzed by smartphone via the imaging platform. The MNA can be tailored specifically to identify specific biomarkers for diagnosis, monitoring, and prognosis of a variety of diseases, which are consumable products.

The technology potentially revolutionizes biomarker detections for point-of-care, monitoring of treatment, and disease progress and has billions of dollars market. It can also connect to a large data processing center in the cloud and meet the high demand of a rising trend of virtual healthcare.

Potential commercial products include:
Sample processing accessories (disposable)
Specific microneedle arrays for different diseases (disposable)
Small “all-in-one” portable devices (maybe a few of them)
Reagents-prefilled microfluidic arrays (disposable)
Gel containing a colorimetric (chromogenic) substrate for horseradish peroxidase (HRP) or alkaline phosphatase (AP) or a fluorescence amplifier like plasmonic fluor, among others (disposable)
Software installed in a smartphone for acquiring and analyzing the data on each MNA (the App is tailored specifically to a specific MNA as a standard biomarker curve is programmed in the software for quantification of specific biomarkers)

Embodiments of the disclosure provide for a simple and accurate detection of multi-biomarkers in blood or any fluidic samples in a laboratory-free manner. A small “all-in-one” portable device, which is referred to herein as “Minidevice for immunoassay” (“miniDia”), is engineered to quantify the biomarkers captured on individual microneedles in a microneedle array (MNA) at home and bedside. The invention takes the advantages of a large surface of a microneedle that can be facilely immobilized with a specific capture antibody, ligand, or aptamer covalently, collectively called capture elements to simultaneously measure a panel of biomarkers on a single MNA in a one-microneedle-one biomarker fashion. We have successfully measured several biomarkers in a single MNA in sera of lupus patients and found that the sensitivity, reliability, and accuracy of the MNA-immunosensors are at least equivalent or 10-times better than the traditional ELISA. We have also fabricated MNA immunosensors for distinguishing bacterial infection from viral infection or diagnosing traumatic brain injury, both of which are ready for validation in clinical samples. A variety of MNA immunosensors can be fabricated similarly to detect a set of biomarkers in 2-3 hrs with a drop of blood samples or any samples as small as 50-100 μl prepared from nasal, throat, oral, and so on. Built on the success of the MNA-immunosensor, we have designed and fabricated a small “all-in-one” portable prototype, miniDia that can process the MNA-immunosensors and acquire and analyze the data on individual microneedles in an automatic or semi-automatic fashion by integrating reagents-prefilled microfluidic capillary array, gel-inked data transfer, and cellphone-based image. The miniDia is engineered to complete all procedures from sample collection to detection results at home, bedside, or battlefield without a laboratory and it represents a billion-dollar market.

Various embodiments of the disclosure provide systems, methods, and apparatus for identifying a plurality of biomarkers in a sample. In some embodiments, the system may include a substrate including a plurality of microneedles (“MNs”) projecting therefrom, referred to herein as a microneedle array (“MNA”). In various embodiments, each MNA may include between 4-1000 MNs and the substrate to which the MNs are attached may range in size between 0.1 cm to 10 cm per side (e.g., as a square or rectangular shape or other shape).

The MNs may have various shapes and may include rounded, tapered, or truncated ends. In certain embodiments in which a sample (e.g., serum obtained from a blood sample) is applied to the MNA, there may be fewer constraints on the shape of the MNs, whereas in those embodiments in which the MNA is inserted into a sample such as skin (e.g., to penetrate the epidermis and obtain information from the dermis or other region) it may be preferable to use MNs which have a tapered or otherwise pointed shape to facilitate insertion into the sample.

In some embodiments, each of the plurality of microneedles may have a plurality of biomarker recognition molecules attached thereto. In various embodiments, the plurality of biomarker recognition molecules may include one or more of antibodies, aptamers, or ligands.

In particular embodiments, each MN, duplicated MNs, or triplicate MNs of the MNA may include biomarker recognition molecules that are directed to a different biomarker than any other MNs of the MNA. Thus, in some embodiments each MN of the MNA only includes biomarker recognition molecules that are directed to one particular biomarker and each MN includes biomarker recognition molecules directed to different biomarkers than all of the other MNs in the MNA.

In some embodiments, the MNA may include a first MN and a second MN where the first MN includes a first plurality of biomarker recognition molecules that are configured to recognize a first biomarker and the second MN includes a second plurality of biomarker recognition molecules configured to recognize a second biomarker different from the first biomarker. Thus, at least two of the MNs of the MNA may have biomarker recognition molecules that are directed to different biomarkers, although other MNs in the MNA may also be directed to the same biomarkers.

In various embodiments, each of the plurality of biomarker recognition molecules is coupled to a respective MN of the MNA using a plurality of dendritic linking molecules. In particular embodiments, each of the plurality of dendritic linking molecules may couple multiple biomarker recognition molecules to the respective MN. As discussed further herein, in one embodiment the dendritic linking molecules may include PAMAM dendrimers.

In certain embodiments, when the MNA is exposed to a sample which includes a plurality of sample biomarkers, each of the plurality of biomarker recognition molecules associated with each of the respective plurality of MNs of the MNA is configured to recognize and couple to a respective biomarker of the plurality of sample biomarkers. After exposure to the sample, each of the plurality of MNs of the MNA is processed to include a labeling compound to identify each of the plurality of sample biomarkers, where the labeling compound may include horseradish peroxidase (HRP) or a fluorescent compound.

In some embodiments, a gel overlay may be provided which contacts the MNA, in a procedure sometimes referred to herein as an “MNA-inked gel assay” (as opposed to a “direct image assay” for reading biomarker testing results by directly imaging the MNA). The gel overlay may include a labeling substrate embedded therein which is configured to form a precipitate within the gel overlay when contacted by the labeling compound. In those embodiments in which the labeling compound is HRP, the labeling substrate may be an HRP substrate such as 3,3′,5,5′-tetramethylbenzidine (TMB) and/or 2,2′-azino-di-[3-ethylbenzthiazoline-6-sulfonic acid](ABTS).

In some embodiments the system may include an imaging adapter that is configured to collect an image from at least one of the entire MNA (e.g., a single image containing all or most of the MNs of the array) or an image of a gel overlay that has contacted the MNA. As disclosed herein, the gel overlay may provide a more convenient way of reading the MNA results and may also provide a higher signal level with less background. In various embodiments, the MNs of the MNA may transfer a reaction product signal to the gel overlay or (e.g., in the case of an enzymatic label such as HRP) the reaction product may be formed within the gel overlay matrix.

In certain embodiments, the imaging adapter may include a phone holder configured to align a camera of a phone (or other camera device, with or without a phone) with an imaging system, where the imaging system may include lenses for projecting an image onto the camera. In some embodiments the imaging system may include at least one lens, a light source, and a specimen holder.

In various embodiments the specimen holder may be configured to hold at least one of the gel overlay or the MNA, which allows the imaging system to collect imaging data from either the gel overlay or the MNA. In one embodiment in which the specimen holder includes the gel overlay associated therewith, the camera of the phone may be configured to obtain an image of the gel overlay using the imaging system.

In some embodiments, the specimen holder may include a mechanical stage that is configured to adjust a position of the at least one of the gel overlay or the MNA to obtain a suitable image. In one embodiment in which the specimen holder includes the MNA, wherein the lenses may include a micro lens and a magnifying lens that are arranged so as to obtain an enlarged image of a MN of the MNA. In addition, the mechanical stage may be configured to adjust the position of the MNA in three dimensions (e.g., laterally/X-Y-directions and/or toward or away from the camera and lenses/Z-direction) such that the camera of the phone obtains an enlarged image of each of the MNs of the MNA. In such embodiments, the mechanical stage may be configured to move (e.g., manually or through an automated process) between images so that each MN of the MNA is within the field of view of the camera.

In various embodiments, the phone holder may further include a disk centrifuge that is configured to process the sample using centrifugal force. For example, the disk centrifuge may spin the sample to separate a test portion of the sample (such as serum) from a remaining portion of the sample (such as blood). In some embodiments, the disk centrifuge may include a circular disk including a sample holder attached thereto in a radial configuration. In particular embodiments, the sample holder may include at least one of a capillary tube holder or a sample channel.

Certain embodiments may further include a biomarker recognition molecule preparation chamber which includes a plurality of microwells. Each of the plurality of microwells may be configured to accommodate a single MN of the MNA to separately attach each of the plurality of biomarker recognition molecules to each of the MNA. Creating separate microwells around each MN of the MNA permits each MN to be linked to biomarker recognition molecules that are directed to a separate biomarker from all of the MNs in the MNA.

In one embodiment, the biomarker recognition molecule preparation chamber may include an overlay (e.g., a piece of double-sided tape having a series of holes arranged to match the MNs of the MNA), where the plurality of microwells may include a plurality of openings extending through the overlay. The overlay may be placed over the MNA and attached to the substrate such that each of the plurality of openings provides a microcontainer for attaching a biomarker recognition molecules to an MN.

In various embodiments, the system may further include an immunoassay station including a microfluidic array that is configured to prepare the sample. In some embodiments, the microfluidic array may include at least one of a sample dilution fluid, a washing buffer, a detection antibody mixture, or a substrate solution disposed therein. In one embodiment, the immunoassay station may further include a manual vacuum system that is configured to draw at least one of the sample dilution fluid, the washing buffer, the detection antibody mixture, or the substrate solution from the microfluidic array to contact the plurality of microneedles.

In certain embodiments, each of the MNs of the MNA may include a tapered end and may include or be made from an optically transparent material. The tapered end of each MN of the MNA may be configured to penetrate at least one of an epidermis or a dermis of a subject. In some embodiments, the system may further include a light source that is configured to deliver light to the subject using each MN of the MNA. In various embodiments, the light source may include a laser including a microlens array that is configured to create a plurality of beams to deliver light to the subject using each MN of the MNA. In certain embodiments, each of the plurality of beams may be transmitted to each MN of the MNA using a respective plurality of fiber bundles, where each fiber bundle of the plurality of fiber bundles may include a central fiber for delivering excitation light surrounded by a plurality of peripheral fibers for collecting emission light.

Various embodiments of the disclosure also provide methods for identifying a plurality of biomarkers in a sample. The method may include providing a substrate including an MNA projecting therefrom, where each of the MNs of the MNA may include a plurality of biomarker recognition molecules attached thereto. The MNA may include a first MN and a second MN where the first MN may include a first plurality of biomarker recognition molecules configured to recognize a first biomarker and the second MN may include a second plurality of biomarker recognition molecules configured to recognize a second biomarker that is different from the first biomarker. The method may also include contacting the MNA with the sample such that at least one biomarker of the plurality of biomarkers in the sample is coupled to at least one biomarker recognition molecule of the plurality of biomarker recognition molecules. The method may additionally include processing the MNA to identify the at least one biomarker of the plurality of biomarkers in the sample.

In some embodiments, each of the plurality of biomarker recognition molecules may be coupled to a respective MN of the MNA using a plurality of dendritic linking molecules, where each of the plurality of dendritic linking molecules may couple multiple biomarker recognition molecules to the respective MN. In some embodiments, the plurality of biomarker recognition molecules may include at least one of antibodies, aptamers, or ligands.

In particular embodiments, the method may further include exposing the MNA to the sample, where the sample may include a plurality of sample biomarkers. Each of the plurality of biomarker recognition molecules associated with each of the respective plurality of microneedles may recognize and couple to a respective biomarker of the plurality of sample biomarkers.

In various embodiments, the method may additionally include processing each MN of the MNA to include a labeling compound to identify each of the plurality of sample biomarkers. In some embodiments, the method may also include contacting the MNA with a gel overlay. The gel overlay may include a labeling substrate that is configured to form a precipitate, such that contacting the MNA with the gel overlay may include contacting the labeling substrate with the labeling compound and forming the precipitate within the gel overlay based on contacting the labeling substrate with the labeling compound. In particular embodiments, the labeling compound may include horseradish peroxidase (HRP) and the labeling substrate may include an HRP substrate.

In certain embodiments, the method may further include collecting, using an imaging adapter, an image from at least one MN of the MNA or from a gel overlay that has contacted the MNA. The imaging adapter may include a phone holder and collecting an image may further include aligning a camera of a phone with an imaging system using the imaging adapter.

In some embodiments, the imaging system may include at least one lens, a light source, and a specimen holder. In various embodiments, the method may further include holding, by the specimen holder, at least one of the gel overlay or the MNA. In certain embodiments the specimen holder may include the gel overlay and collecting an image may further include obtaining, using the camera of the phone, an image of the gel overlay using the imaging system.

In one embodiment, the specimen holder may include a mechanical stage and the method may further include adjusting a position of the at least one of the gel overlay or the MNA using the mechanical stage.

In some embodiments the specimen holder may include the MNA and the at least one lens may include a micro lens and a magnifying lens. In particular embodiments of the method, collecting an image may further include obtaining, using the micro lens and the magnifying lens, an enlarged image of a MN of the MNA; adjusting, using the mechanical stage, the position of the MNA in three dimensions; and obtaining, using the camera of the phone, the enlarged image of each MN of the MNA based on adjusting the mechanical stage.

In certain embodiments of the method, the phone holder may further include a disk centrifuge. In some embodiments, prior to contacting the MNA with the sample, the method may further include processing the sample using centrifugal force to separate a test portion of the sample from a remaining portion of the sample. In various embodiments, the disk centrifuge may include a circular disk including a sample holder attached thereto in a radial configuration. In some embodiments, the sample holder may include at least one of a capillary tube holder or a sample channel.

In particular embodiments of the method, providing a substrate including an MNA may further include providing the substrate which includes a biomarker recognition molecule preparation chamber including a plurality of microwells. Each of the plurality of microwells may be configured to accommodate an MN of the MNA to separately attach each of the plurality of biomarker recognition molecules to each MN of the MNA. Some embodiments may further include providing the substrate including the biomarker recognition molecule preparation chamber and an overlay, where the plurality of microwells include a plurality of openings extending through the overlay and where the overlay may be placed over the MNA and attached to the substrate such that each of the plurality of openings provides a microcontainer for attaching a biomarker recognition molecules to an MN.

In some embodiments, providing a substrate including an MNA may further include providing the substrate which includes an immunoassay station including a microfluidic array and preparing the sample using the microfluidic array. In one embodiment, the microfluidic array may include at least one of a sample dilution fluid, a washing buffer, a detection antibody mixture, or a substrate solution disposed therein. In various embodiments the immunoassay station may further include a manual vacuum system and preparing the sample using the microfluidic array may further include drawing at least one of the sample dilution fluid, the washing buffer, the detection antibody mixture, or the substrate solution from the microfluidic array to contact the MNA.

In particular embodiments, each MN of the MNA may include a tapered end and each MN of the MNA may include an optically transparent material. The method may further include penetrating at least one of an epidermis or a dermis of a subject using the tapered end of each of the plurality of microneedles. In other embodiments, the method may further include delivering, using a light source, light to the subject using each MN of the MNA.

In various embodiments, delivering light to the subject may further include delivering light to the subject using the light source, where the light source may include a laser which includes a microlens array that is configured to create a plurality of beams to deliver light to the subject using each MN of the MNA.

In some embodiments, delivering light to the subject may further include transmitting each of the plurality of beams to each MN of the MNA using a respective plurality of fiber bundles. Each of the plurality of fiber bundles may include a central fiber for delivering excitation light surrounded by a plurality of peripheral fibers for collecting emission light.

Some embodiments provide software, for example a smartphone app or other software associated with a local or remote computing system for controlling components, transmitting and saving data, and processing raw data (e.g., images) to generate results. The software may be stored on a computer-readable storage medium such as a non-transitory computer-readable medium. The non-transitory computer-readable medium may have stored thereon instructions that, when executed by the processor, cause a processor (e.g., a processor of a smartphone or other computing device) to execute at least a portion of the methods described herein. Light or other data obtained from the MNA may also be stored on the non-transitory computer-readable medium. The non-transitory computer-readable medium can be local to the computing device or may be remote from the device, so long as it is accessible by the processor.

In various embodiments, the software may include instructions to carry out any of the methods disclosed herein, e.g., for collecting and/or processing data obtained using the MNAs. In various embodiments, the software may include instructions for processing the plurality of microneedles to identify the at least one biomarker of the plurality of biomarkers in the sample which further include: obtaining an amount of each biomarker of the plurality of biomarkers associated with each of the plurality of microneedles, comparing the amount of each biomarker of the plurality of biomarkers associated with each of the plurality of microneedles to a reference data set, and quantifying a level of each biomarker of the plurality of biomarkers associated with each of the plurality of microneedles based on comparing the amount of each biomarker of the plurality of biomarkers to the reference data set (e.g., a standard curve that may be specific to a particular MNA or category of MNAs). In certain embodiments, the software may include instructions for at least one of presenting or transmitting information identifying the level of each biomarker of the plurality of biomarkers associated with each of the plurality of microneedles to a user (e.g., a patient, a clinician, a researcher, etc.). In various embodiments, the software may be or include a smartphone app.

Embodiments of the disclosure are disclosed further below:

### Materials:

Poly(methyl methacrylate) (PMMA) (Mw˜120000), poly(ethyleneimine) (PEI) (Mn˜60000; Mw˜750000), ethyl acetate, PAMAM dendrimer (ethylenediamine core, generation 4.0 solution), 1-Ethyl-3-(3-dimethylaminopropyl)carbodiimide (EDC), N-hydroxysuccinimide (NHS), suberic acid bis(3-sulfo-N-hydroxysuccinimide ester), double-stranded DNA (dsDNA), albumin methylated from bovine serum (mBSA), and bovine serum albumin (BSA) were purchased from Sigma-Aldrich (Waltham, MA, USA). SeramunBlau spot dark 3,3′,5,5′-Tetramethylbenzidine (TMB) substrate was obtained from Seramun Diagnostica GmbH (Heidesee, Germany). Streptavidin-horseradish peroxidase (HRP) was purchased from Abcam. Polydimethylsiloxane silicone (PDMS) elastomers base and curing agent (SYLGAR 184 Silicone Elastomer Kit) were obtained from Dow (Midland, MI, USA). PBS was purchased from Life Technologies (Carlsbad, CA, USA). Human C-reacted protein (CRP) was obtained from Lee Biosolutions (Maryland Heights, MO, USA). RP conjugated C-reactive protein antibody was purchased from Novus Biologicals (Littleton, CO, USA). EnzMet RP detection kit was obtained from Nanoprobes (Yaphank, NY, USA). Carboxyl group conjugated CRP aptamer (/5Carboxyl/TTTTTGGCAGGAAGACAAACACGATGGGGGGGTATGATTTGATGTGGTT GTTGCATGATCGTGGTCTGTGGTGCTGT (SEQ ID NO: 1)) was purchased from Integrated DNA Technologies (Coralville, IA, USA). Human IFN-α 2a capture antibody, human IFN-α 2a, and biotinylated human IFN-α 2a detection antibody were obtained from PBL assay science (Piscataway, NJ, USA). Human procalcitonin (PCT), Human insulin-like growth factor binding protein-2 (IGFBP-2), Human sTNF RII/TNFRSFlB, and Human VCAM-1/CD106 DuoSet ELISA kits were purchased from R&D System (R&D Systems, Minneapolis, MN, USA). Distilled water was obtained by using a Millipore Milli-Q ultrapure water purification system (Burlington, MA, USA). Vivid™ plasma separation membrane was purchased from Fisher Scientific (Hampton, NH, USA).

Clinical samples: Serum samples from Lupus patients (N=42) and healthy controls (N=34) were provided by University of Houston, Houston, Texas. The samples were collected under an institutional approved IRB protocol. All samples were aliquoted and stored at −80° C. until use.

### Fabrication of MNA and Surface Modifications

A PDMS-MNA was fabricated as previously detailed. 3 To fabricate a transparent MNA using PMMA in place of PDMS, PDMS elastomer base solution mixed with curing agent at a 10:1 ratio was poured into a well of a 6 well plate, and mixed well, followed by centrifuge at 2,000 rpm for 10 min to remove bubbles. A PDMS-MNA was placed into the mixture, and bubbles were removed under vacuum. The mixture was then heated at 85° C. for 3 hours and the PDMS MNA was peeled after cooling to obtain a female MNA mold. To the female MNA mold, PMMA of 1 mL solution was added, followed by centrifugation at 4,000 rpm for 15 min. The PMMA solution was prepared by dissolving at 20 g/100 mL ethyl acetate and stirring at 78° C. for overnight prior to its addition to the female mold. The casting process occurred at 80° C. for 4 h to remove ethyl acetate, followed by adding 1 mL of PMMA solution to cover the first layer of dried PMMA at 85° C. overnight. The process was repeated once.

After PMMA was dried and cooled, the resultant MNA was peeled from the PDMS mold, and immersed in PEI solution (10% v/v) at 60° C. and stirring for 6 hours. PAMAM dendrimers with ethylenediamine core was conjugated onto the PEI-modified surface of each microneedle in the MNA via suberic acid bis(3-sulfo-N-hydroxysuccinimide ester) sodium salt as a cross-linking reagent to conjugate aptamer or antibodies. Following dendrimer modification, two alternative methods were employed to conjugate capture antibody or aptamer on each MN. EDC and NHS were first dissolved in MES buffer (10 nM) and then capture antibody or carboxyl group-conjugated aptamer was mixed with EDC and NHS coupling agent solution to covalently conjugate it onto the targeted microneedles via EDC/NHS coupling reaction, allowing reaction for 5 hours. A washing buffer (PBS contained 0.05% Tween-20) was then added to wash the MNA every 5 min for a total of three times to remove unreacted reagents. Non-specific binding on the MNA was blocked by 2% skimmed milk at 36° C. for 1 hour and then washing every 5 min for a total of three times. For the detection of anti-ds-DNA antibody, the targeted microneedles were incubated mBSA solution, followed by addition of ds-DNA. The steps of washing and blocking non-specific binding were similar as described above. These MNAs with specific capture elements immobilized on individual MNs in the array are named MNA immunosensors.

### Immunoassays for Quantification of Multi-Biomarkers on Individual Microneedles in the Array

Two MNA-immunosensors were developed to sufficiently acquire and analyze the signals on individual microneedles by Smartphone. A specific MNA-immunosensor was either incubated with patients' serum or a known antigen at varying concentrations, followed by 3× washes. The immunosensor was then subject to detective antibody that was linked to biotin, fluorescence, or HRP either directly or indirectly. Similar to sandwich immunoassay, the precipitant substrate of HRP or fluorescence amplifier was added to the MNA and the colorimetric substrate deposited on each microneedle was photographed and analyzed directly, called “direct image assay” that is suitable for detecting a few biomarkers. Secondly, the colorimetric or fluorescent amplifier was embedded in a gel, onto which the MNA immunosensors “ink”, with which the signal on a 3D microneedle was transferred into a 2D gel for much more convenient and efficient acquisition of signals by cellphone. This assay is named as “MNA-inked gel assay” and can accommodate as many biomarkers as needed.

### ELISA Assay for Comparison

Serum CRP, IGFBP-2, TNF-RII, VCAM-1 levels of the patients with lupus and healthy controls were measured using commercial ELISA kits. The ELISA assays were conducted per the manufacturer's instructions. The results were read on a UV spectrophotometer (Epoch, Biotek, Winooski, VT, USA). The data were analyzed by GraphPad Prism 7.

Design and fabrication of an immunoassay station in miniDia

Apart from the rubbers, spring, LED, battery, and lens, all the other components of the miniDia were designed by utilizing a Solidwork 2017 and 3ds Max 2015, converted to STL formation, and printed by a 3D printer. Some complicated and tiny components of the portable platform were printed by commercial 3D printing service companies. Commercially available rubbers were first test for their durability and integrated them into the miniDia device.

### Results and Discussion

Built upon our previous success in the MNA-based sampling of blood biomarkers via laser-pretreated skin, we continuously developed functionalized MNAs aimed at measurement of a panel of biomarkers, rather than a single biomarker, for onsite diagnosis and monitoring. These functionalized MNAs are called MNA-immunosensors or aptasensor and generated via a new platform that can be applied to a variety of analytes or biomarkers for diagnosis, monitoring, and prognosis of various diseases. Three specific MNA-immunosensors and one aptasensor have been engineered and investigated for distinguishing viral infection from bacterial infection or monitoring lupus, traumatic brain injury, or cocaine ingestion. We also designed and fabricated a miniDia capable of processing and analysis of these MNA-immunosensor or aptasensor onsite without the need of a laboratory.

### A Novel Platform to Generate MNA-Immunosensors

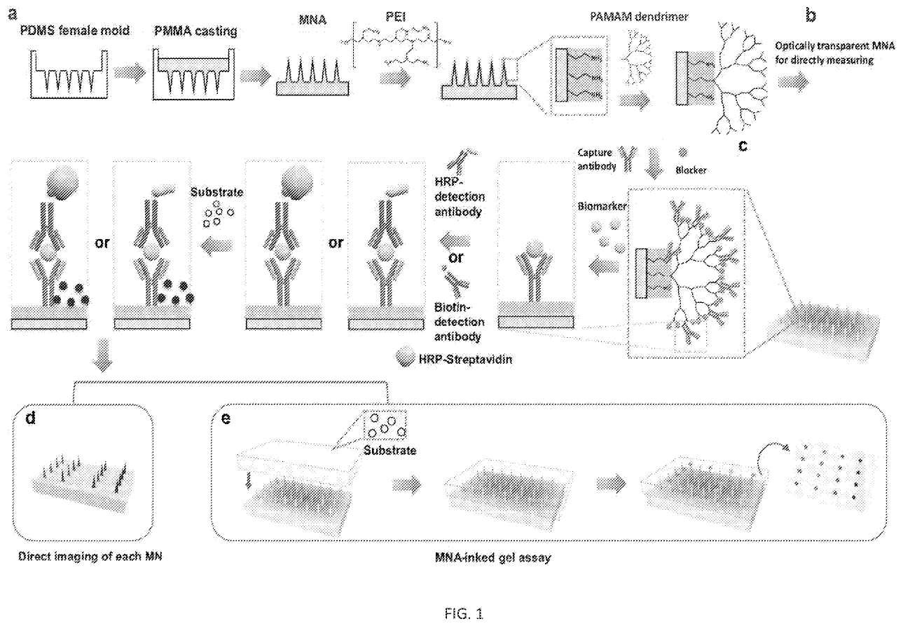

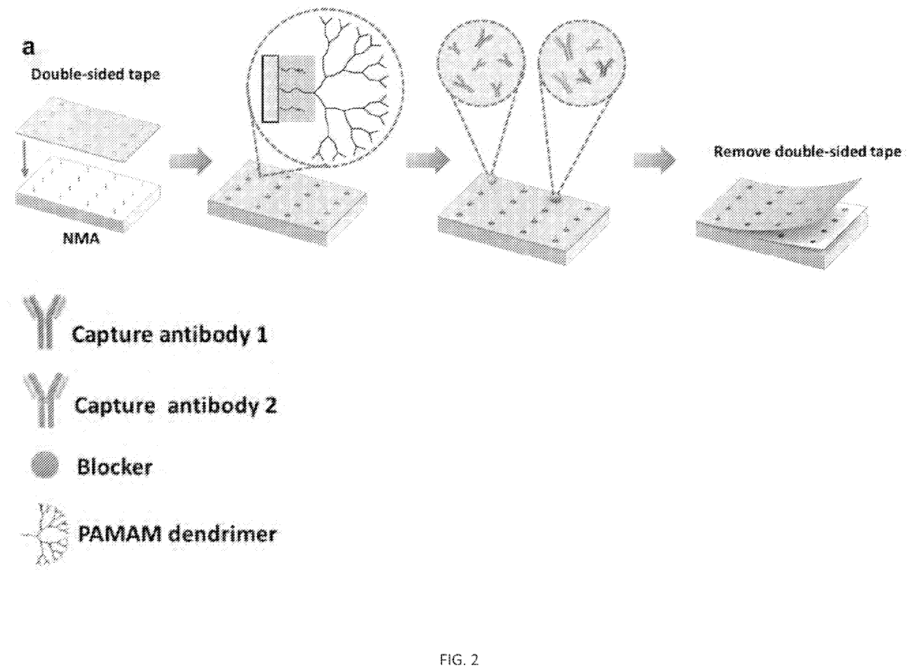

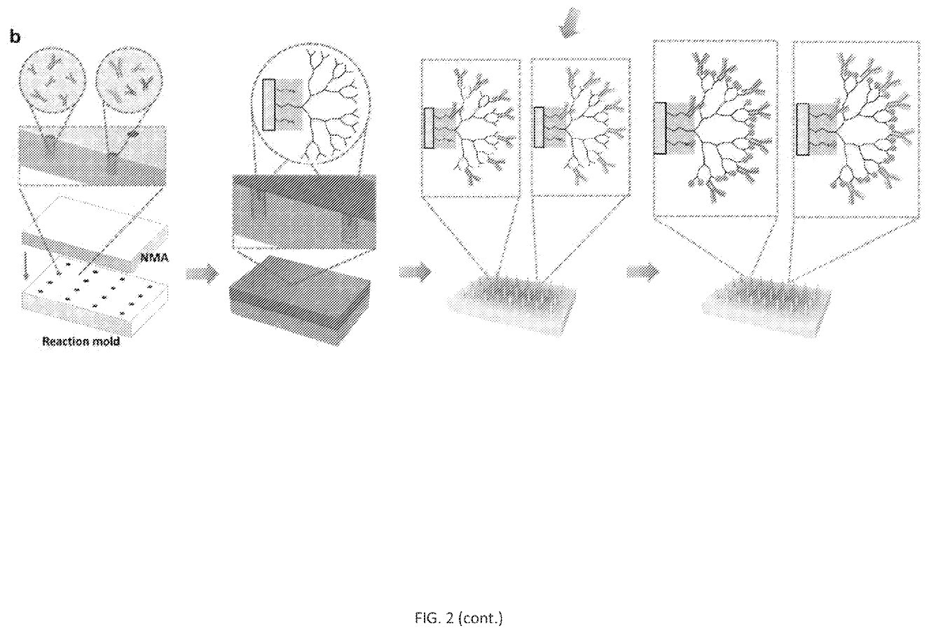

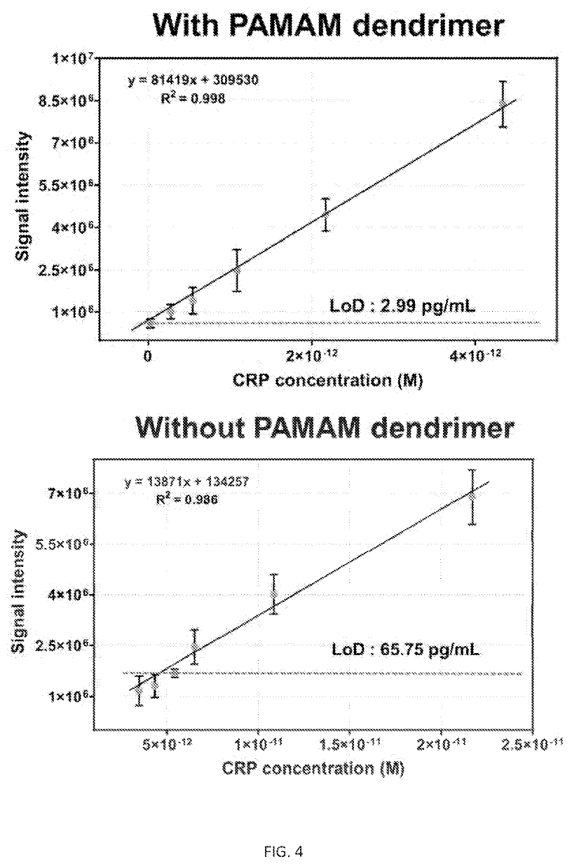

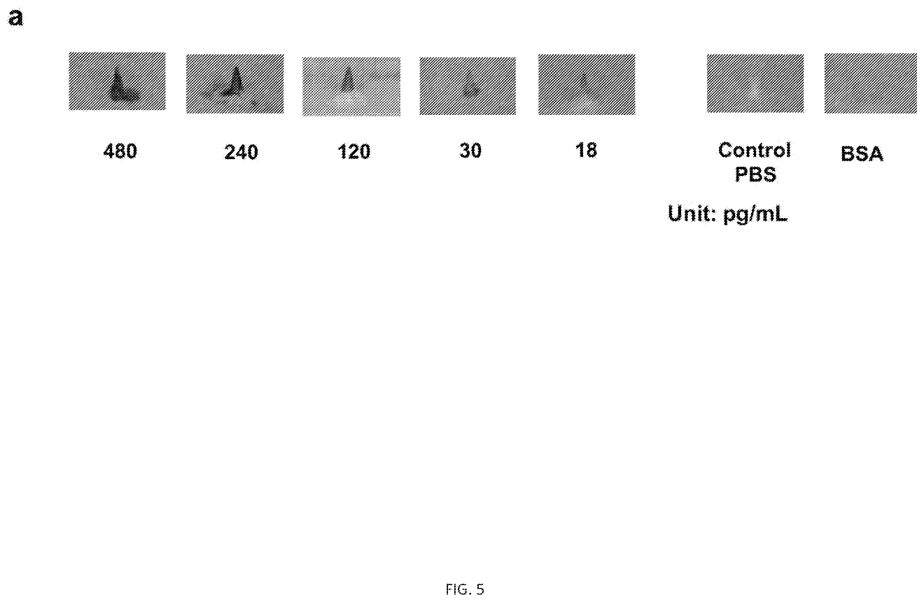

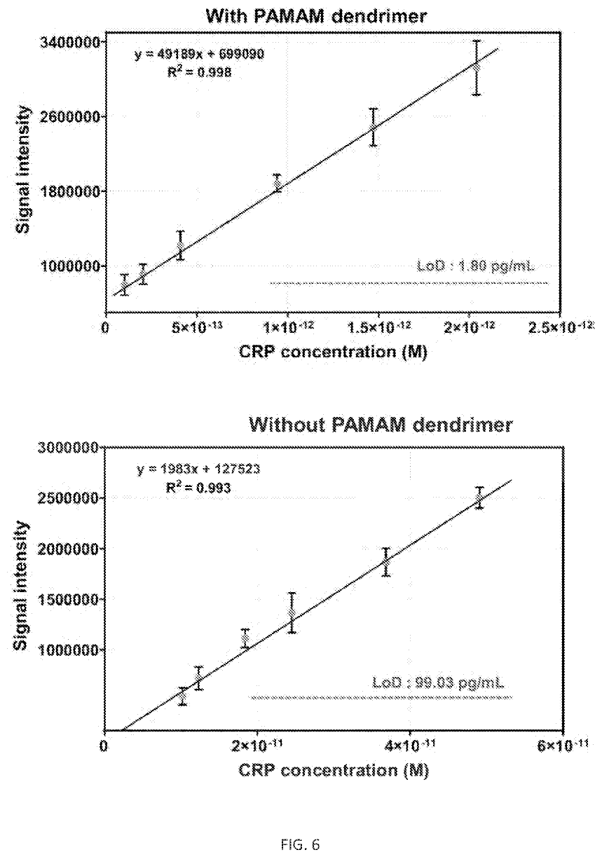

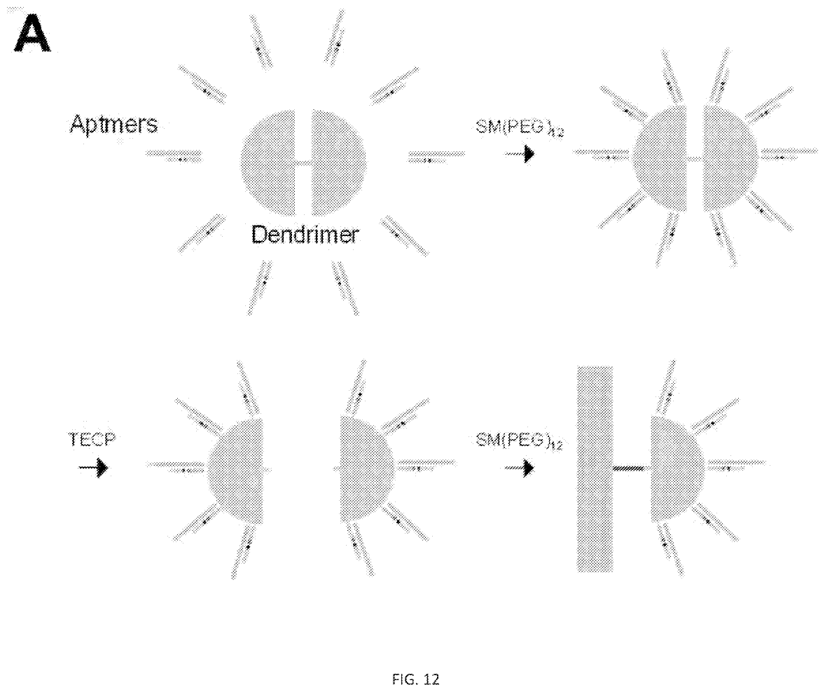

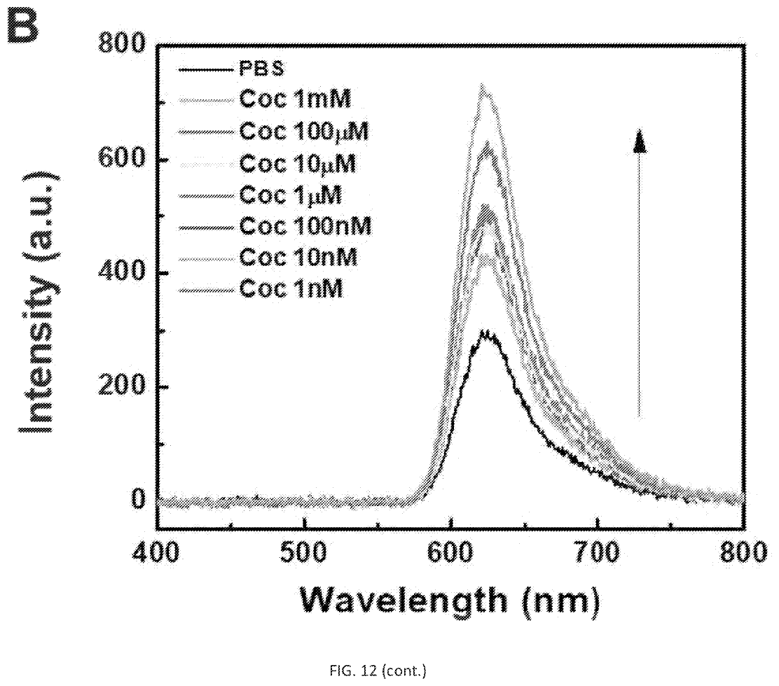

As shown in FIG. 1, a light transparent MNA was first fabricated from a PDMS female mold by casting crystally clear PMMA material (FIG. 1a). The PMMA MNA surface was then covalently modified by PAMAM dendrimers to magnify the binding sites on individual microneedles (MNs) in the array (FIG. 1b). The modification increased the detection sensitivity by 20-folds, 55-folds, or 5000-folds as shown in FIG. 4, FIG. 6, and FIG. 12B, respectively. The capture antibody was subsequently linked to the branches of the dendrimer via two alternative novel approaches so that a specific antibody could be covalently linked to a tar get MN in the MNA in a one antibody-on-one microneedle fashion. First, the MNA base was covered by a double-side tape that was pre-treated with laser to punch an array of tiny holes precisely aligning with the microneedles in the MNA (FIG. 2a). These tiny holes function as a microcontainer to hold a specific antibody solution surrounding each MN (FIG. 2a). Second, a PDMS MNA female mold was slightly modified by replacing the microneedle shape with a cylinder shape using a 3D printer (FIG. 2b). Each of the cylinders functions as a micro-container and can be filled by a specific antibody solution (FIG. 2b). These two approaches not only make it possible to mount many different capture elements in a single MNA in a one-antibody-on-one-microneedle fashion, but also effectively minimize non-specific background signals on the MNA base, which is highly significant for enhancing the specificity and accuracy of the assay. These innovations are critical for multiplex detection of biomarkers, in contrast to the MNA-based assays under development or clinical or preclinical studies that can detect only a single biomarker on one MNA.

After conjugation of specific antibodies in individual MNs in the MNA, the MNA could be processed similarly as traditional ELISA but with 50× less reagent solutions, including biomarker binding, detective antibody reaction to an epitope distinct to the one recognized by the capture antibody (FIG. 1c). The colorimetric or fluorescent substrate could be directly deposited on each microneedle, photographed, and analyzed directly by Smartphone, called “direct microneedle image assay” (FIG. 1d). The direct microneedle image assay is suitable for detecting a few biomarkers and requires fewer steps as compared to MNA-inked gel assay” described in the following. Distinguished from direct microneedle image assay, the “MNA-inked gel assay” is based on embedding the colorimetric or fluorescent amplifier in a gel (FIG. 1e). When the MNA bearing HRP- or Biotin-detective antibody is inserted into the substrate-saturated gel, the signal on a 3D microneedle is converted into a 2D gel for much more convenient and efficient acquisition of signals by smartphone (FIG. 1e). This MNA-inked gel assay can accommodate as many biomarkers as needed, offering more consistent analysis of the binding signals on each microneedle in the MNA.

In brief, there are three innovations in this new platform: First, we are the first to conjugate a specific antibody in a single microneedle in the array, enabling many antibodies to be conjugated on an MNA in a one-antibody-on-one microneedle fashion; Second, greatly amplifying the binding signals by dendrimer modification of the surface of each microneedle; and third, the MNA-inked gel assay allows conversion of the signal on a 3D microneedle into a 2D gel for much more convenient and efficient acquisition of signals by smartphone.

Distinguishing Bacterial Infection from Viral Infections by MNA-Inked Gel Assay

Many bacterial and viral infections manifest similar and overlapping clinical symptoms such as fever and are difficult to diagnose. Clinicians often prescribe antibiotics immediately to avoid a potential risk of severe and possibly life-threatening bacterial infections, especially in low- or middle-income countries where the result of confirmative bacterial cultures would not be available until 48 to 72 hrs later and the golden window of antibiotic treatment would be missing terribly by then, leading to sepsis. Sepsis kills millions of people each year globally and disables millions more, in part owing to a delay in diagnosis and treatment. This leads to unnecessary antibiotic uses, greatly contributing to the growing public health crisis of antibiotic resistance. The cost of treating antibiotic resistance-related illness is huge today. For instance, management of methicillin-resistant Staphylococcus aureus (MRSA) alone estimates $30 billion per year in the USA. A point-of-care diagnosis with 100% sensitivity for bacterial infections and a high specificity for viral infections can substantially slow down the development of multi-drug resistant (MDR) microbes and greatly minimize the risk of sepsis saving millions of lives.

In the past decade, a set of biomarkers have been well identified and extensively investigated in clinics to distinguish bacterial infection from viral infection. One group has found that interferon (IFN) signaling pathway would be activated specifically for viral infection after examining 47,300 probes hybridized with RNA samples covering the whole human transcriptome from 30 febrile children and 35 afebrile children. IFNα-2a is contributed to innate antiviral immunity against viruses through upregulation of antiviral genes. Various viral infections would result in the increase of IFNα-2a, yet bacterial infections or acute inflammation in patients do not (at a body temperature≥38.5° C., with a specificity of 0.92). In contract to IFNα-2a, numerous clinical studies have shown that procalcitonin (PCT) production doesn't rise significantly with viral or non-infectious inflammations, and has been regarded as the best biomarker for bacterial infection with a sensitivity of 90% (≥0.5 ng/mL) or 100% (≥0.2 ng/mL) depending on the cutoff level. In addition, CRP, an inflammatory biomarker, is routinely used to indicate the severity of inflammation in acute conditions such as serious bacterial infections of the lung or skin or coinfection or inflammation causing fever.

These three biomarkers alongside a control BSA were investigated in the gel inked MNA colorimetric assay in an attempt to distinguish bacterial infection from viral infections in the emergency room. We first evaluated a pair of capture and detective antibodies for each of these biomarkers and compared the results between the NIA-based assay and traditional ELISA. To this end, various concentrations of human CRP, mouse CRP, IFNα-2a, PCT, or BSA solutions were prepared in PBS solution containing 2% BSA as capture antibodies, and then added onto each microneedle in the MNA as described above, followed by 2 hr incubation at room temperature (RT). Mouse CRP and BSA were used as negative controls to confirm the specificity. The MNA was rinsed with washing buffer every 5 min for a total of three times to remove the non-specific binding. The detective antibodies for CRP, IFN-α 2a, and PCT were HRP-conjugated anti-human CRP antibody, biotinylated human IFN-α 2a or PCT antibody, which were added to the MNA sequentially and incubated for I hour, followed by incubation with or without streptavidin-HRP for 20 minutes. The MNA was then washed every 5 min for a total of three times. Gel was tailored to a size of the MNA, air dried, and then immersed with colorimetric substrate solution (SeramunBlau spot dark TMB substrate) until it was saturated. The MNA was inserted into the gel and pressed firmly for 15 minutes, after which the gel was removed and washed using distilled water and carefully transferred to a plastic dish for imaging. The images of stained gel were captured by a microscope and analyzed by ImageJ.

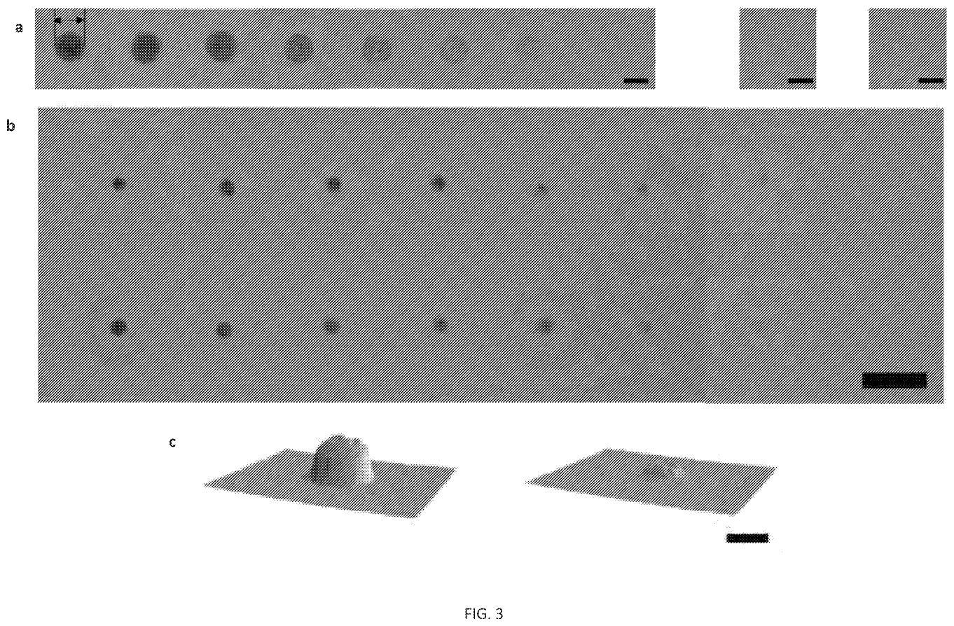

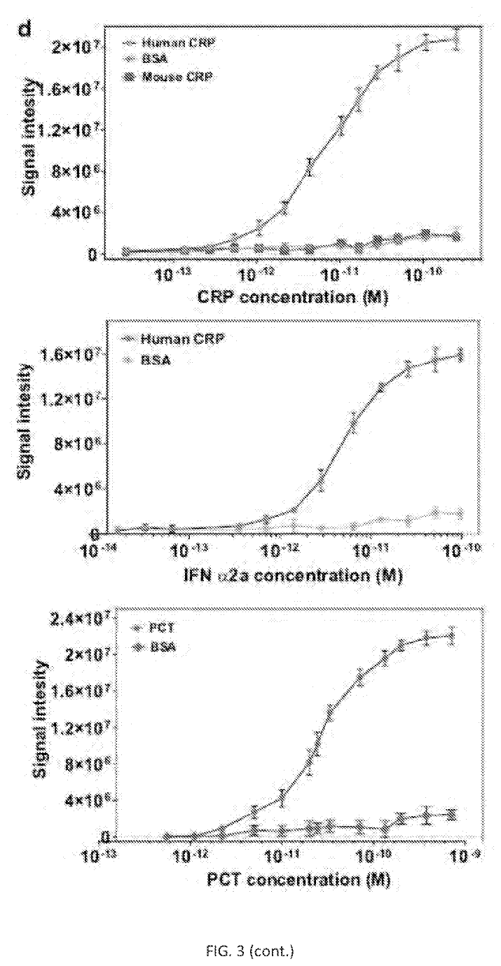

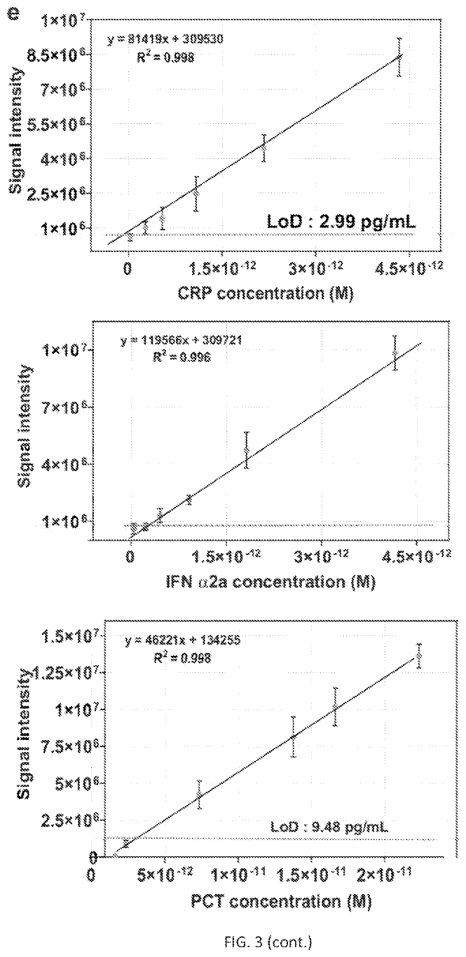

A representative gel image of one MN row was given in FIG. 3a and the spots on the gel corresponded to human CRP in a range from 0.925 to 75 pg/mL in PBS compared to negative controls 75 pg/mL BSA and PBS alone. The image is enlarged from FIG. 3b that showed duplicate samples of each concentration of the biomarker in two rows of the MNA confirming reproducibility and specificity. Due to the cone shape of the microneedles, the spots were mostly of a round shape with an approximate diameter of 237 μm and their color intensities and sizes are proportional to the human CRP concentrations in the test region, in good agreement with the results of 3D images of stained spots on gel in FIG. 3c. To quantify the color intensity, the image mode was converted to “grayscale” and the corresponding intensity of each spot was plotted with the known concentration to establish the binding kinetics and standard curves (FIGS. 3d and 3e). FIG. 3d shows the dose-dependent responses of MNA colorimetric assays on gel to indicated concentrations of human CPR, IFNα-2a, and PCT in PBS. FIG. 3e exhibits the calibration curves of CRP, IFNα-2a, and PCT of MNA colorimetric assay on gel, which are used to identify the limit of detection (LOD). LOD for human CRP, IFNα-2a, PCT, could reach 2.9874 pg/mL (1.2962×10⁻¹³ M), 3.5077 pg/mL (1.8231×10⁻¹³ M), and 9.48 pg/mL (6.7771×10⁻¹³ M) in PBS, respectively, and the linear ranges are from 0.625 to 100 pg/mL, 0.4 to 80 pg/mL, 10 to 300 pg/mL, respectively. For detection of CRP and PCT, this assay confers a superior sensitivity to the most of commercial ELISA kits (LOD of CRP ELISA from ABCam: 4 pg/mL; LOD of CRP ELISA from R&D: 22 pg/mL; LOD of PCT ELISA from Raybiotech: 30 pg/mL). For detection of IFNα-2a, the sensitivity of this assay is also competitive with commercial ELISA kit (LOD of CRP IFNα-2a from PBL: 1.92 pg/mL). The results are summarized in Table 1. Another major modification of the MNA surface was decoration of dendrimers that possesses a myriad of primary amine groups on the surface of each microneedle, with which the activated conjugation sites on each microneedle for capture antibody were dramatically increased. Consequently, the sensitivity of the MNA-immunosensor with dendrimers increases significantly, 20× higher with dendrimer modification than that without dendrimer (FIG. 4).

TABLE 1
Functionality comparison of commercial ELISA
kits with MNA colorimetric assays on gel

| Assay | LOD | | Linear Range | |
|---|---|---|---|---|
| Human C-Reactive Protein/ | 0.022 | ng/mL | 0.8-50 | ng/mL |
| CRP Quantikine ELISA Kit | | | | |
| (R&D) | | | | |
| Our work | 0.00299 | ng/mL | 0.625 to 100 | pg/mL |
| PBL assay science IFN-α-2a | 1.92 | pg/mL | 1.95-124 | pg/mL |
| ELISA kit | | | | |
| Our work | 3.5 | pg/mL | 0.4-80 | pg/mL |
| Ray biotech PCT ELISA kit | 30 | pg/mL | 30-20000 | pg/mL |
| Our work | 9.48 | pg/mL | 10-300 | pg/mL |

Of note, the clinical cut-off for CRP, IFNα-2a, and PCT level is 1.0 mg/L, 6 pg/mL, and 0.25 ng/mL, respectively, all of which are higher than the LOD values of our assay, confirming that the sensitivity of the MNA immunosensor meets the clinical application.8,9 Moreover, the signal intensity for the assay was at baseline levels in the presence of the negative controls (mouse CRP and BSA), confirming a high specificity of the MNA colorimetric assay. We are actively seeking sera from various infected patients to validate the assay in comparison with ELISA side-by-side.

### Monitoring Lupus by Direct Imaging of Each Microneedle in the MNA

We next fabricated and investigated a specific MNA-immunosensor to assay serum samples from patients with lupus and healthy controls. Systemic lupus erythematosus is a chronic and inflammatory autoimmune disease presenting various abnormalities in a wide range of organs for different patients. It requires a panel of serum biomarkers for better diagnosis so that false positive or false negative results can be averted. The disease is also required for continuously monitoring during the treatment to guide further therapy. Immune disorders cause a large number of varying types of autoantibodies capable of attacking body's normal tissues or organs, such as anti-double-stranded DNA antibody (anti-dsDNA), in patients with active lupus, leading to the formation of immune deposits and vascular inflammation in many organs. During this process, inflammatory molecules stimulate endothelial cells to express E-selectin that binds to carbohydrate groups of leucocytes. As a result, leucocytes adhere to endothelium and up-regulate adhesion molecules such as vascular cell adhesion molecule-1 (VCAM-1) for facilitating transmigration of inflammatory cells. Some researchers have reported that the VCAM-1 levels in patients with active lupus were elevated. Although the exact mechanisms underlying the increased human insulin-like growth factor binding protein-2 (IGFBP-2) in lupus are unknown, some have found significantly higher levels of IGFBP-2 and tumor necrosis factor receptor type II (TNF-RII) in patients with active lupus over the control groups, suggesting that they could be the biomarkers of active lupus. IGFBPs might be also involved in IGF-1-dependent or IGF-1-independent signaling pathways to regulate immune cell proliferation. TNF-RII, which is expressed by T lymphocytes, can inhibit activations of tumor necrosis factor α (TNFa) that is regarded as a protective role in lupus. It participates in regulation of B-cell activation and related production of autoantibodies, a hallmark for the development of lupus.

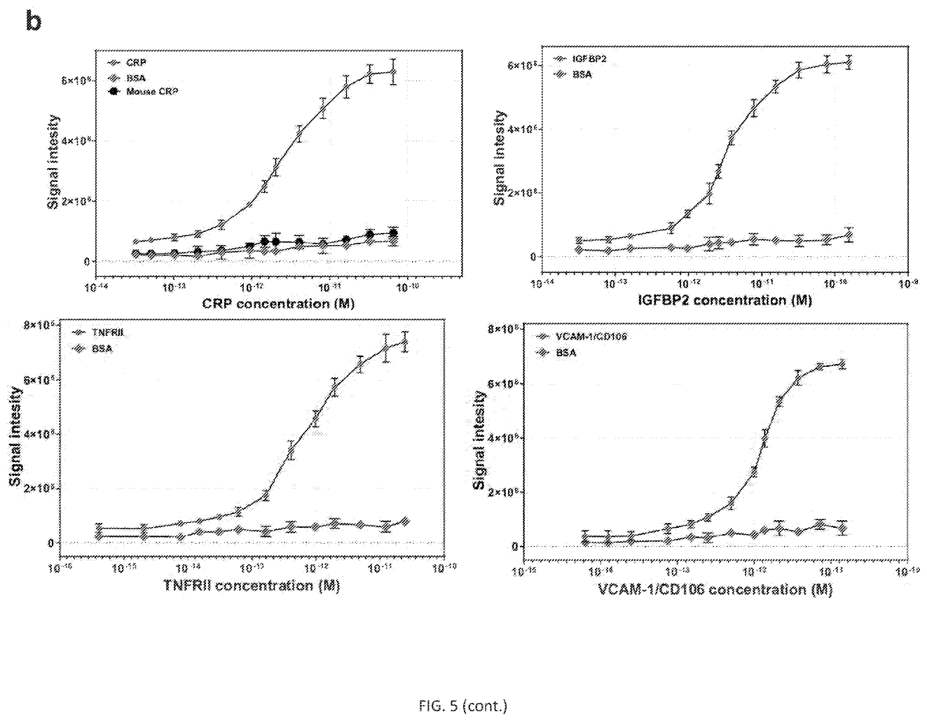

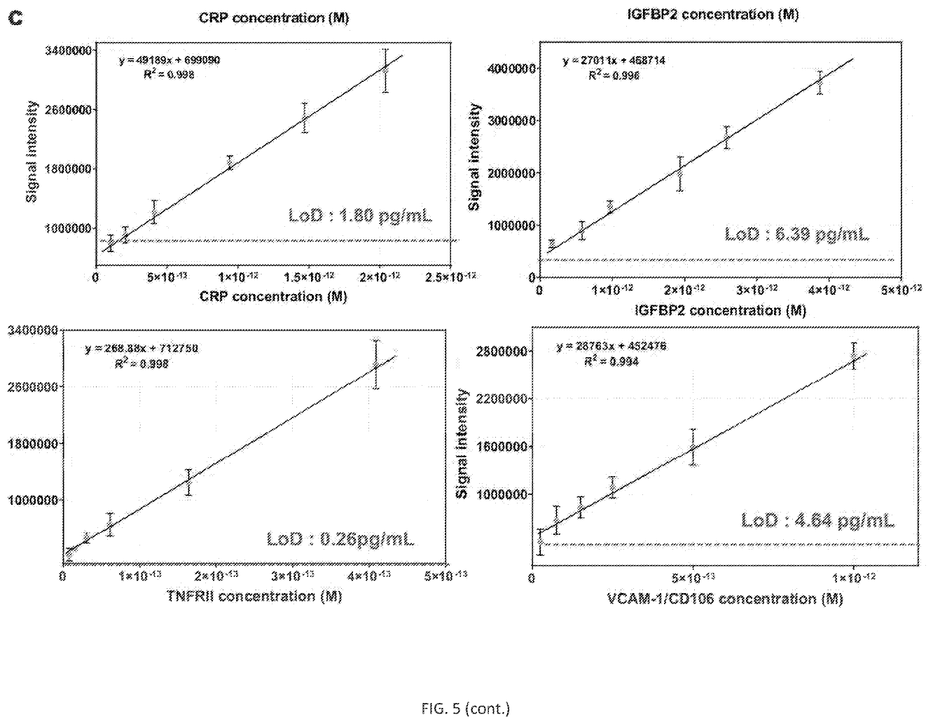

These five biomarkers (human CRP, IGFBP-2, TNF-RII, VCAM-1, and anti-dsDNA antibody) were selected for the investigation. Similar to the MNA functionalization process described above, MNA were incubated with a series of concentrations of capture antibodies directed at human CRP, mouse CRP, IGFBP-2, TNF-RII, VCAM-1, or bovine serum albumin (BSA) solutions in PBS containing 2% BSA for 2 hours, followed by 3× washes to remove non-specific binding. Detective antibodies were HRP-conjugated anti-human CRP antibody and biotinylated human IGFBP-2, TNF-RII, or VCAM-1 antibody, which were sequentially added onto each microneedle in the array and incubated for 1 hour, followed by 3 washes. The MNA was then reacted with streptavidin-HRP for 20 minutes and washed. Substrate solution A, B, and C (EnzMet HRP detection kit) were then successively added onto the MNA and mixed every 2 min in PBS. FIG. 5a shows representative images of individual microneedles capturing human IGFBP2 from 18 to 480 pg/mL and 200 pg/mL of BSA. The color intensity on the surface of each microneedle in the array was proportional to the concentrations of IGFBP2 in the test region. In FIG. 5b, shown are the dose-dependent responses of the colorimetric assays on the microneedles to indicated concentrations of human CRP, IGFBP-2, TNF-RII, or VCAM-1. The calibration curves of human CRP, IGFBP-2, TNF-RII, and VCAM-1 offer the LOD of the corresponding biomarkers 1.8 pg/mL (2.06×10-13 M), 6.4 pg/mL, 0.26 pg/mL (1.09×10-14 M), and 4.6437 pg/mL (5.8×10-13 M), respectively, and the linear ranges from 2.5 to 50 gp/mL, 5 to 120 pg/mL, to 10 pg/mL, or 2 to 80 pg/mL, respectively, (FIG. 5c). Table 2 summarizes the comparison of commercial ELISA assays of human CRP, IGFBP-2, TNF-RII, VCAM-1 with the results of our MNA immunosensors. Clearly, in comparison with the commercial ELISA kits, the LOD values of our colorimetric assays are an order of magnitude lower than those of ELISA kits, suggesting at least Ox higher sensitivity than ELISA in addition to a 50× reduced amount of reagents used for cost-effectiveness. Again, microneedle surface decoration with dendrimers increased the sensitivity of the assay by about 55-fold (FIG. 6). Furthermore, the high specificity of the assay was demonstrated by negligible signal on the microneedles immobilized with negative controls mouse CRP and BSA.

TABLE 2
Comparison of commercial ELISA kits with colorimetric
assays on individual MNs of an MNA

| Assay | LOD | | Linear Range | |
|---|---|---|---|---|
| Human C-Reactive Protein/CRP | 0.022 | ng/mL | 0.8-50 | ng/mL |
| Quantikine ELISA Kit (R&D) | | | | |
| Our work | 0.0018 | ng/mL | 2.5-50 | pg/mL |
| Human IGFBP-2 Quantikine | 90 | pg/mL | 300-2000 | pg/mL |
| ELISA kit (R&D) | | | | |
| Our work | 6.39 | pg/mL | 5-120 | pg/mL |
| Human TNFRII Quantikine | 2.3 | pg/mL | 7.8-500 | pg/mL |
| ELISA kit (R&D) | | | | |
| Our work | 0.26 | pg/mL | 0.18-10 | pg/mL |
| Human VCAM-1/CD106 | 0.06 | ng/mL | 0.3-20 | ng/mL |
| Quantikine ELISA kit (R&D) | | | | |
| Our work | 0.00464 | ng/mL | 2-80 | pg/mL |

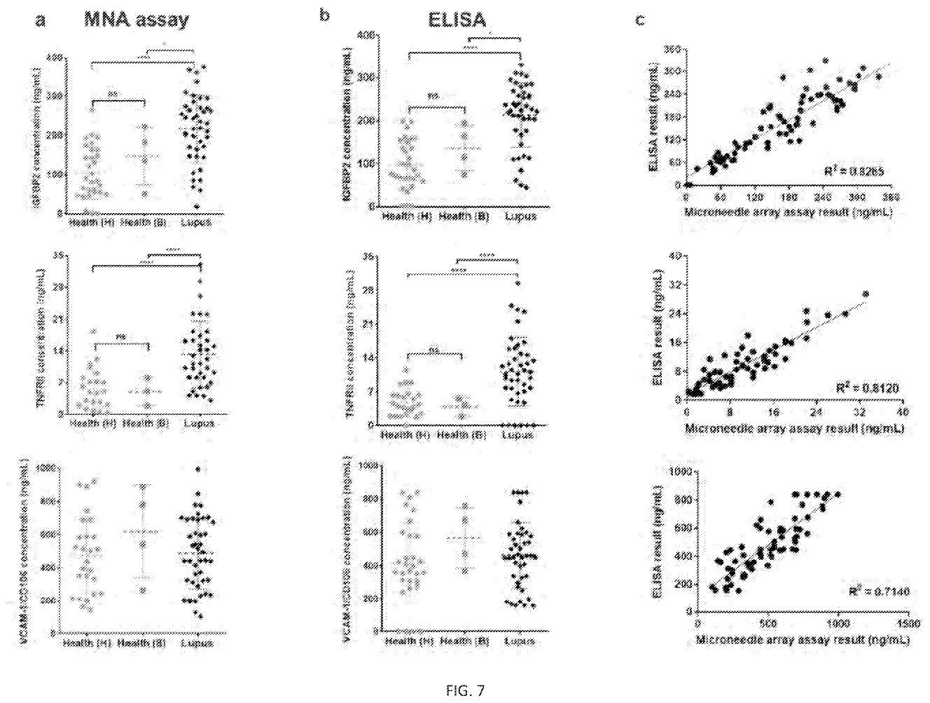

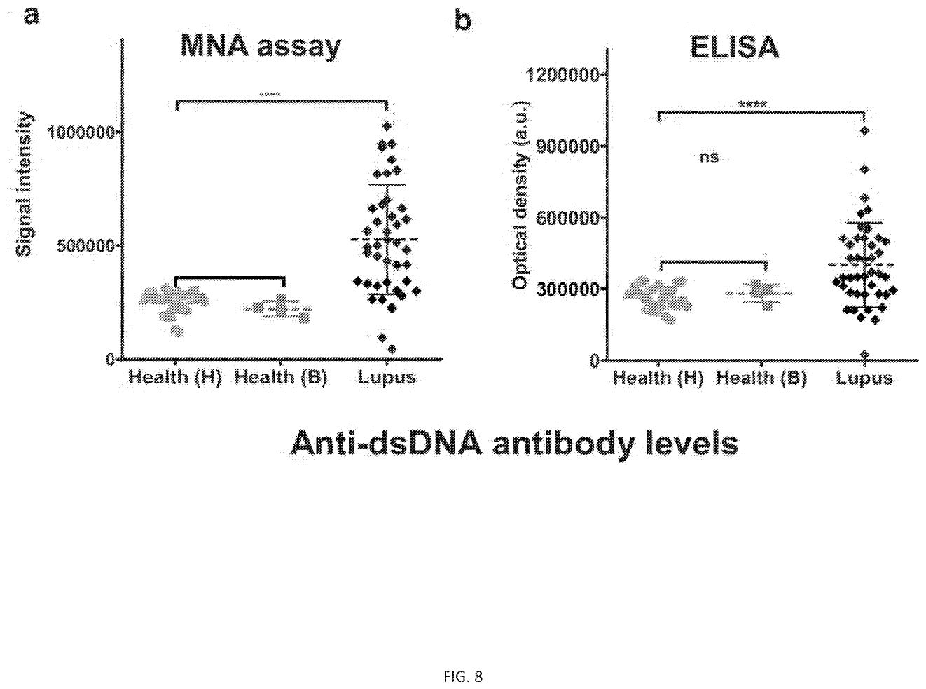

Serum samples collected from patients with lupus (N=42) and healthy controls (N=34) were next evaluated by the MNA-immunosensor to validate the clinical potential of colorimetric assays. ELISA was run in parallel for comparisons. The data are shown in FIGS. 7-8, highly correlation of the two assays and similarity of the two results. Thus, the same conclusion can be drawn from the two assays: i.e. in comparison with healthy controls, levels of IGFBP2, TNF-RII and anti-dsDNA antibody rose significantly in the serum of patients with lupus and especially, the level of TNF-RII and anti-dsDNA antibody were significantly higher in patients with lupus than that in healthy controls. However, there was no significant difference in the level of VCAM-1 between lupus patients and healthy controls.

A paired correlation analysis of the results between ELISA and the MNA immunosensors showed strong corrections with R² values of 0.8265, 0.8120, and 0.7140, for IGFBP2, TNF-RII, and VCAM-1 detection, respectively, suggesting that the reliability, sensitivity, and accuracy of the MNA immunosensors are comparable to clinically ELISA kits.

An optically transparent MNA for real-time measuring cocaine and its derivative in blood

Illicit drug use has risen substantially across the US over the past decade, wherein cocaine, one of the most addictive and harmful drugs, plays an inglorious role. It is estimated that the annual economic impact from cocaine misuse alone is $442 billion. Abuse of cocaine and other drugs yearly incurs over $740 billion in the cost to the national economy. There is no cookie-cutter solution to end illicit drug use, but many common-sense measures lessen the impact of. As such, a rapid and reliable screening assay, especially, the real time measurement of cocaine, may stop any sample manipulation considerably, which is a serious issue for urine samples. We have shown substantial accumulation (>10,000) of circulating biomarkers in the epidermis and upper dermis following brief green laser irradiation of skin because the green laser (532-590 nm) is preferably absorbed by hemoglobin (Hb) and oxygenated Hb (oxyHb) inducing capillary dilation and biomarker extravasation beneath the laser-treated skin. When a functionalized MNA is applied to the laser-pretreated skin, biomarkers extravasated from the skin capillary can bind to the capture elements on the MNA so that blood biomarkers can be detected without drawing any blood. Moreover, the MNA can stay in the skin for an extending period of time for real time measurement of biomarkers if necessary. However, the light penetration efficiency via the intact skin varies considerably with skin colors. For instance, black skin can block the light transmission substantially, jeopardizing the biomarker measurement.

### Light Transparent MNA

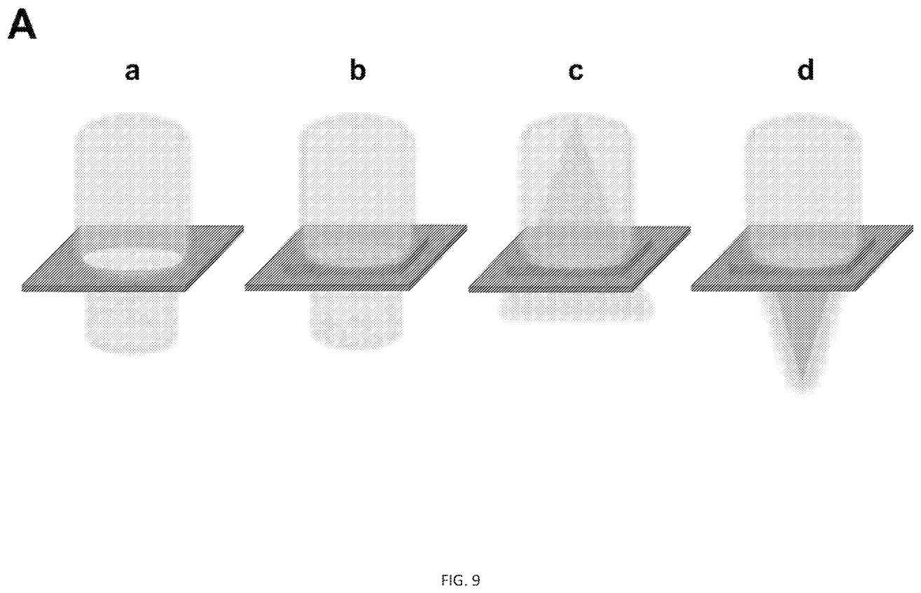

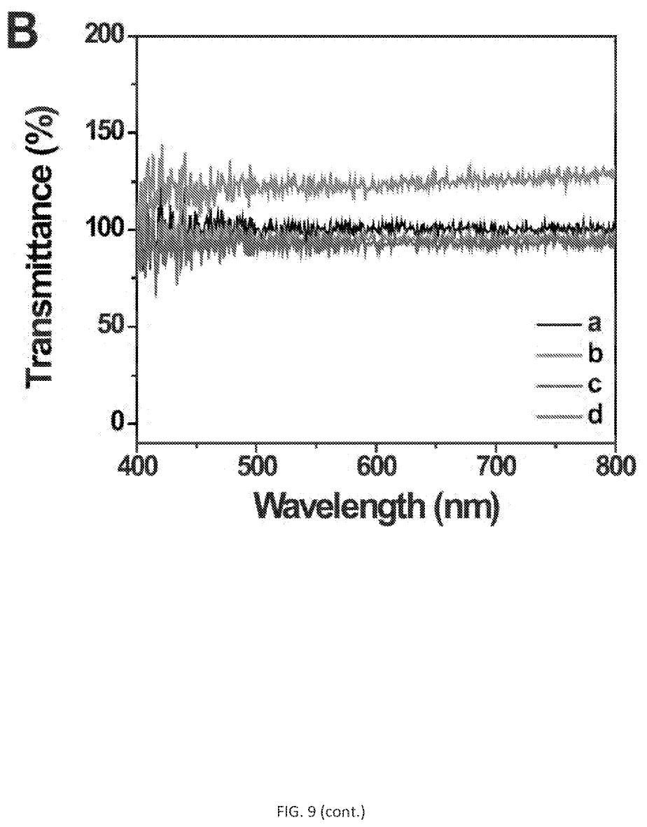

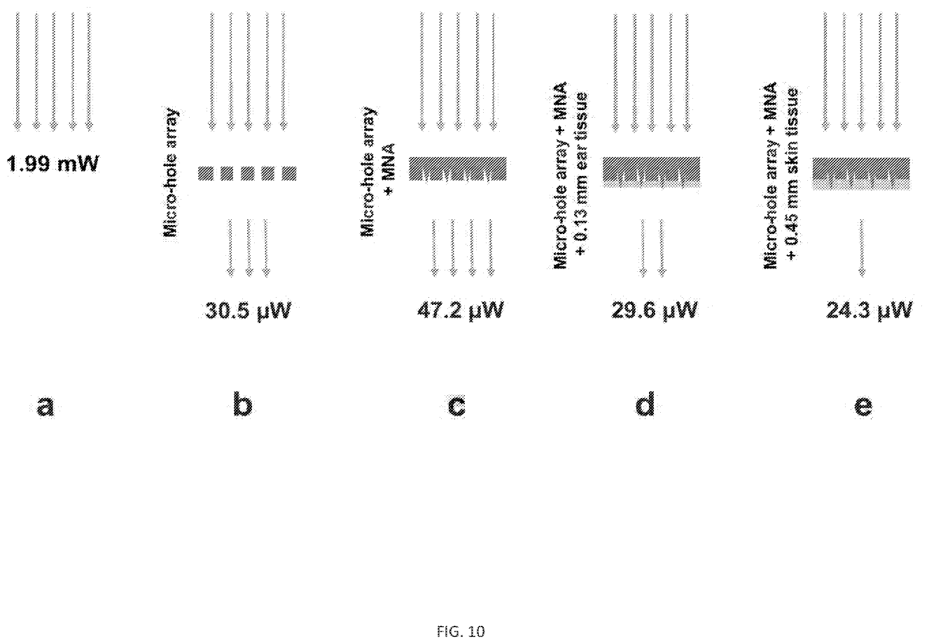

To circumvent the adverse impact of skin color on laser-induced extravasation, we fabricated optically transparent MNA with polymethyl methacrylate (PMMA) as described in FIG. 1a. The optically transparent MNA can direct light to the upper dermis bypassing the epidermal layer. To evaluate the light transmittance of the MNA, we first compared its transmittance in the presence (gray) or absence (black) of 1-mm-thick PMMA-synthesized MNA base (no microneedle in both situation) (FIG. 9A) where the transmittance efficiency sets 100% arbitrarily in the absence of the MNA base (FIG. 9a and FIG. 9B, black trace), slightly lower in the presence of the MNA base ring than its absence (compare structures of FIG. 9b vs. FIG. 9a). Against all expectations, the light penetration rate through an optically transparent MNA was significantly increased, rather than decreasing, relative to the 100% baseline (FIG. 9a vs. FIG. 9d and red in FIG. 9B). An increase of the light transmission by the transparent MNA is presumably due to the cone-shaped microneedle that acts as a concave lens (FIG. 9d). The concave lens' effect was proven because inversion of the microneedle reduced the light penetration (FIG. 9c and blue in FIG. 9B). The enhancement was demonstrated consistently over light spectra from the visible (400-700 nm) to near-infrared (700-800 nm) wavelengths (FIG. 9B). To assess an efficiency of light penetration of the skin via the MNA, MNA penetrated a 0.45-mm-thick mouse skin or a 0.13-mm-thick mouse ear tissue and then the light was administered on top (FIG. 10). The light transmission efficiency in the presence of MNA and the ear skin tissue was comparable to or only slightly lower than that obtained in the absence MNA or ear skin tissue (FIG. 10d vs FIG. 10b), owing to the enhanced transmission by the MNA (FIG. 10b vs. FIG. 10c). Increased tissue thickness reduced the light transmission as anticipated (FIG. 10e.) Conceivably, as the MNA penetrates skin and bypasses the epidermic layer to direct the light to upper dermis, the light transmission is likely to be independent on the skin color.

### Cocaine Detection (In Vitro)

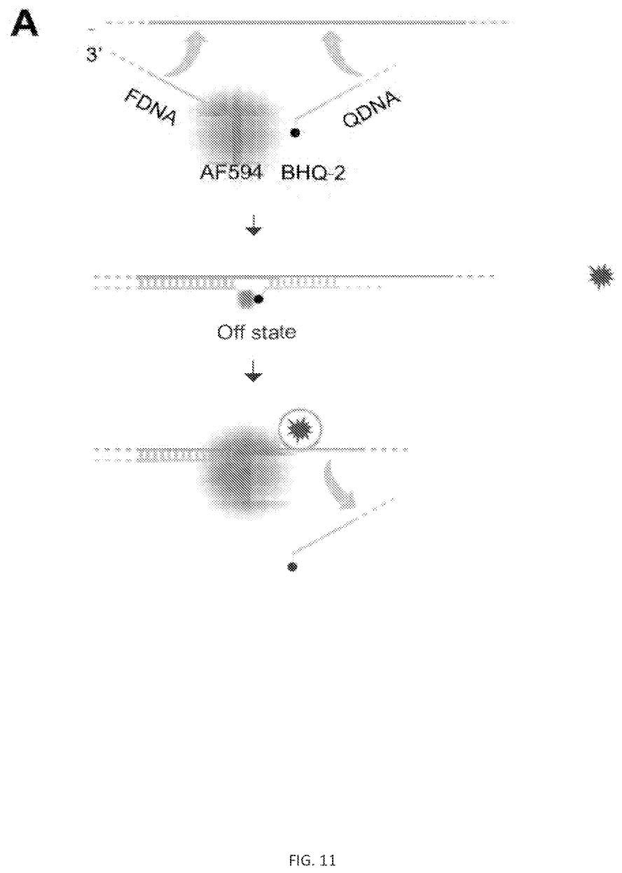

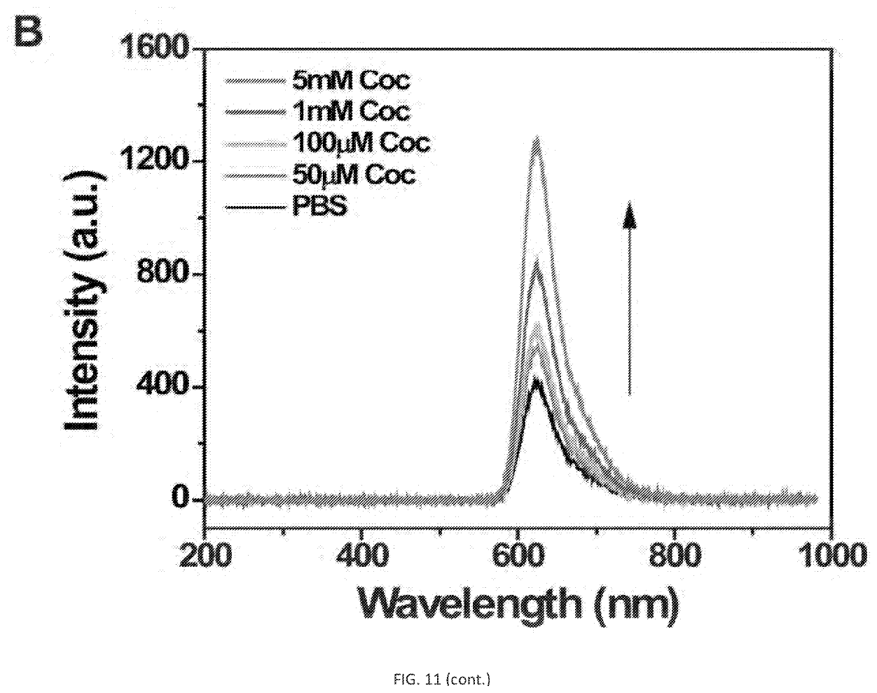

We developed a “Signal-On” fluorescent aptasensor to sensitively and specifically detect both cocaine and its major metabolite benzoylecgonine (BZE). The design takes advantages of cocaine-binding aptamer to create a DNA-cocaine complex as well as a DNA-DNA duplex with complementary DNA sequence. A DNA duplex is composed of an extended DNA strand (StemDNA) partially hybridized to complementary fluorophore-conjugated DNA (FDNA) in the 3″ end and Black Hole Quencher™ (BHQ-2) labeled QDNA in the other side, which leads to a low fluorescent signal, due to proximity of the fluorophore (Alexa Fluor 594) to the quencher (the “OFF” state). When cocaine is introduced, the structural transition of StemDNA occurs so that QDNA is displaced to form the DNA-cocaine complex, giving rise to a significantly higher fluorescent signal (the “ON” state; FIG. 11A). As such, the cocaine-binding signal is endowed onto the MNA by immobilizing the DNAs on its surface. However, this MNA-based aptasensor displayed a limit of detection (LoD) 50 μM, which was not sufficient for clinical measurement of cocaine (FIG. 11B). To improve the sensitivity, dendrimers were covalently attached on the surface of the MNA to amplify the signals as described in FIG. 1a. As shown in FIG. 12A, the aforementioned Coc-aptasensor (FIG. 11A) was coupled to a PAMAM dendrimer via chemical crosslinking using SM(PEG)24; second, the disulfide bond of cystamine core was cleaved with a mild redox reagent of Tris(2-carboxyethyl)phosphine (TCEP), generating two DNA-conjugated thiol dendrons; and third, the dendrons are immobilized on the MNA surface by chemical crosslinking using SM(PEG)24. Dendron-modification greatly increased the sensitivity and resulted in an astonishing LoD value of 10 nM for cocaine detection, a 5,000× increase (FIG. 12B). Hence, our innovative MNA-aptasensor provides a robust and easy-to-use platform for cocaine detection with a high sensitivity and selectivity.

An optical fiber for delivering excitation light and collecting emission light from a single microneedle

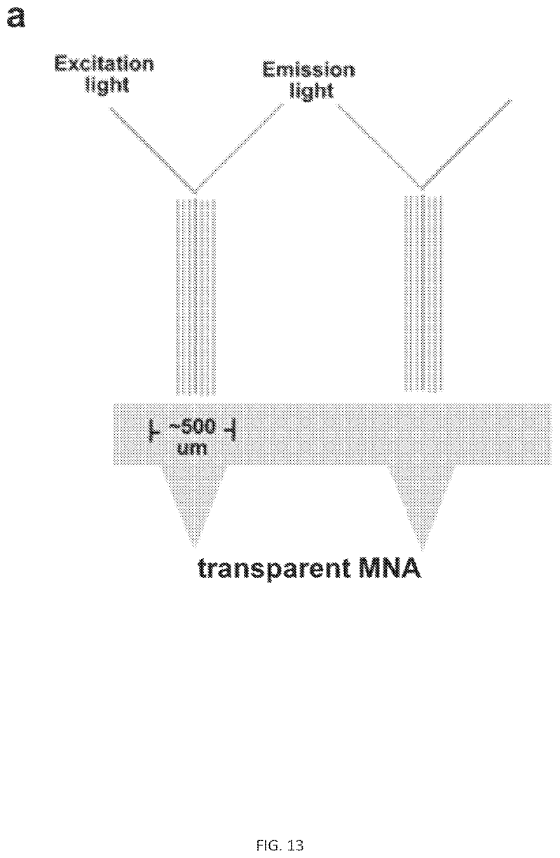

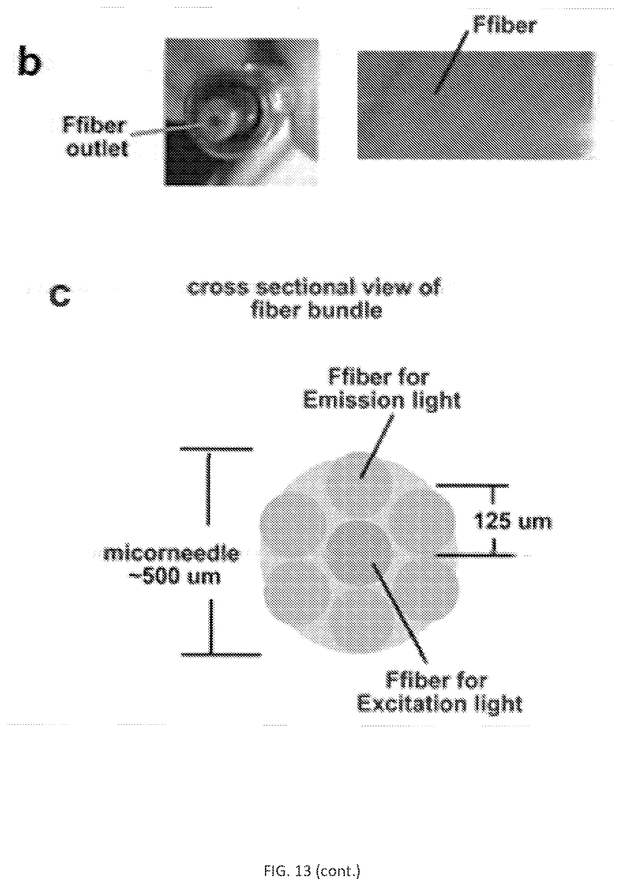

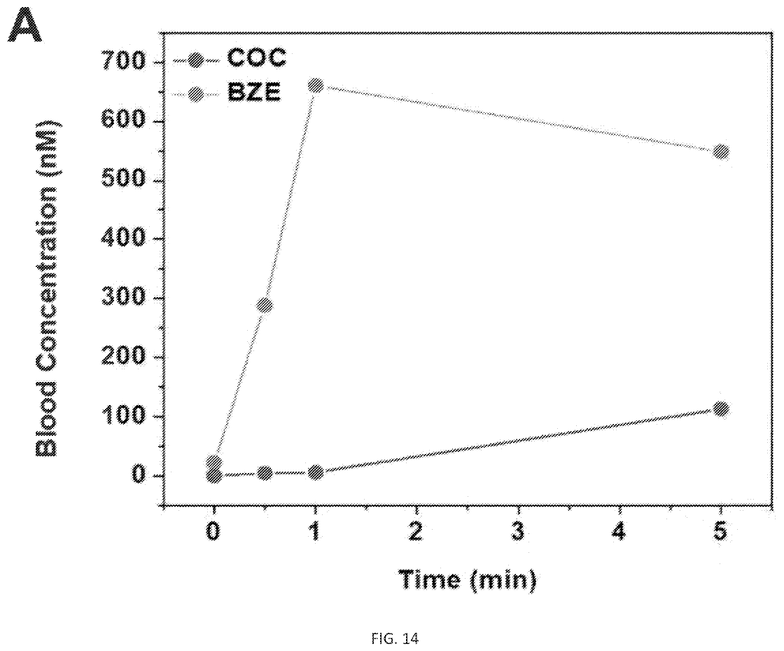

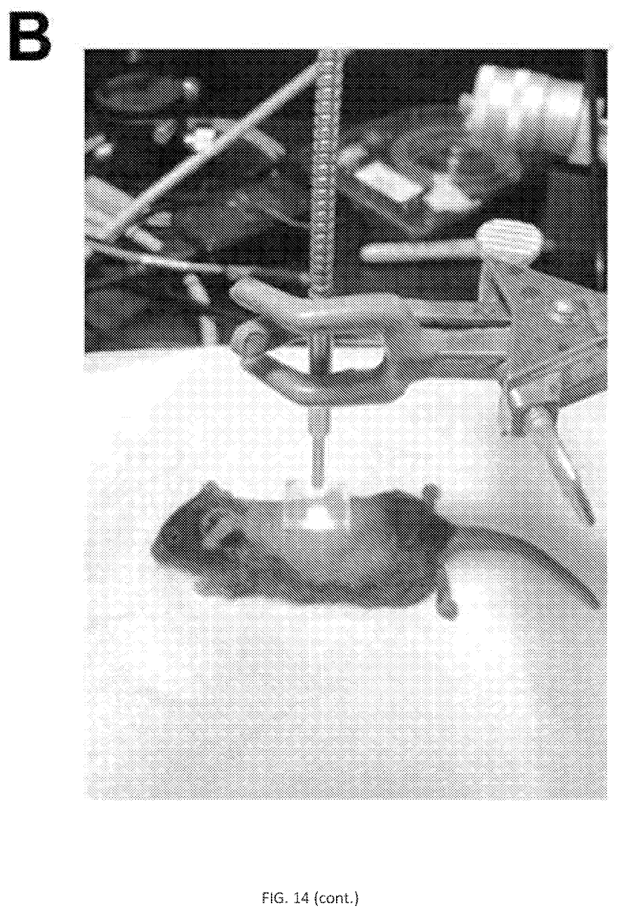

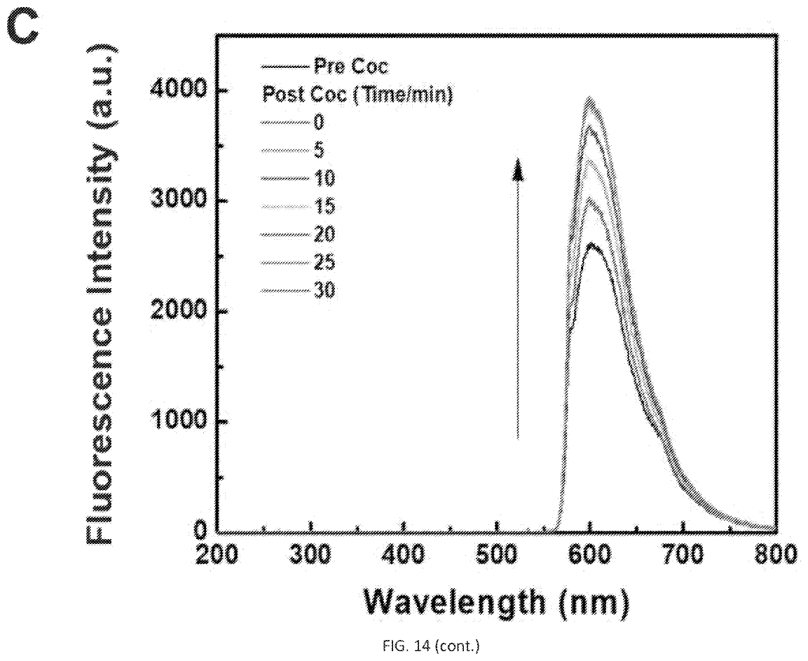

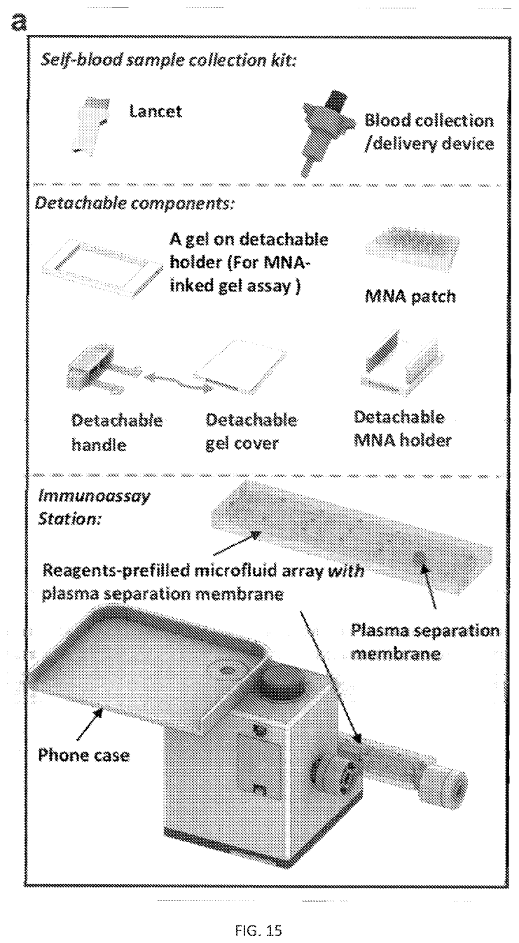

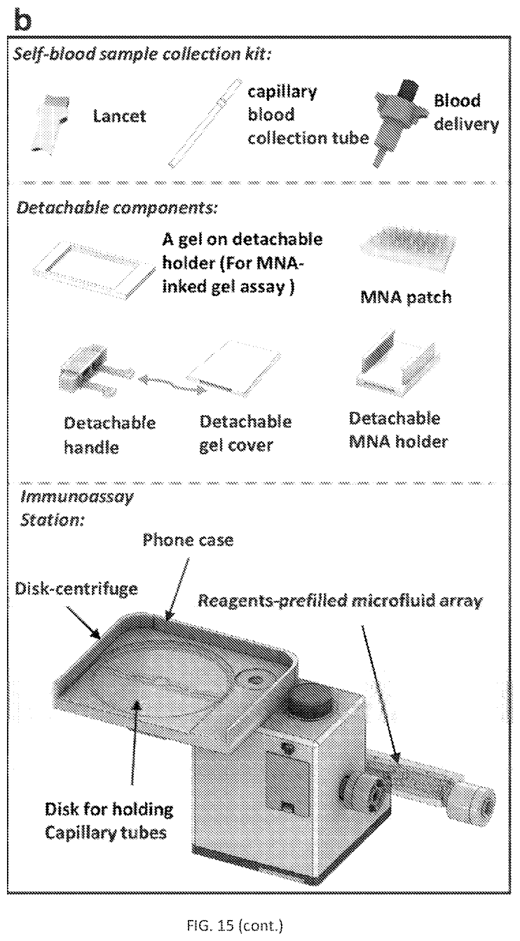

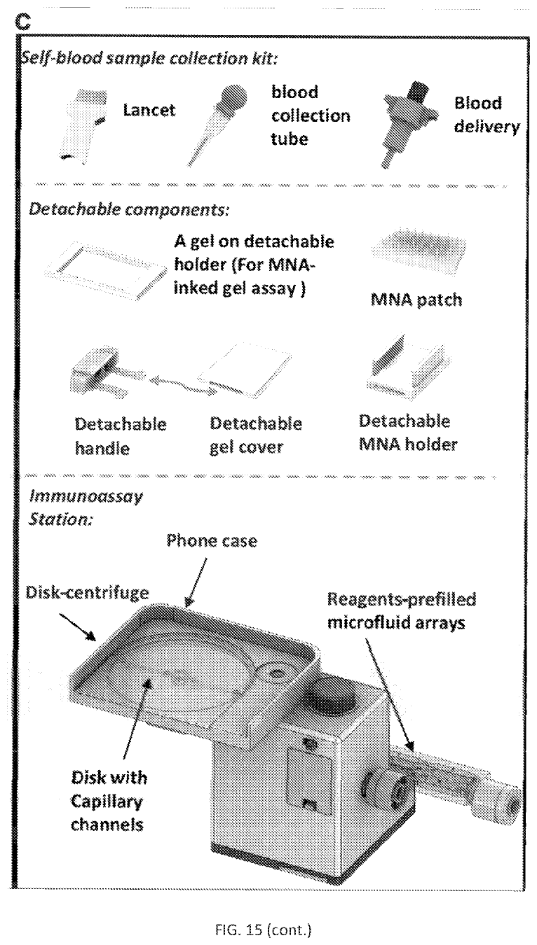

An optical fiber of 500 um in diameter was engineered including 6 emission light collection fibers each at 125 um in diameter, surrounding the single excitation light fiber at the center (FIGS. 13a & 13c). A photo of the fiber is shown in FIG. 13b. The excitation laser (532-589 nm) is administered directly to the upper dermis via the fiber and transparent MNA to induce skin capillary leakage or extravasation of biomarkers. The six light collection fibers are significantly more effective than a single collection fiber and greatly enhance the sensitivity of cocaine detection. To validate this, cocaine was IP administered at 1.5 mg/Kg per mouse and blood was sampled at 0, 0.5, 1, and 5 min, post-injection. Pharmacokinetic study showed that cocaine (blue line) concentration slightly increased over the 5 min, as it was rapidly metabolized into BZE that was increasing sharply at the first 1 min and then stayed in plateau (red line) (FIG. 14A). When the MNA aptasensor was inserted into the black B6 mice receiving 1.5 mg/kg cocaine intraperitoneally and illuminated with 532 nm laser on the dorsal skin (FIG. 15B), a significant growth of fluorescence intensity as a function of time due to displacement of QDNA by cocaine was clearly seen on individual microneedles (FIG. 15C), confirming the ability of the aptasensor to detect the illicit drug in real time. The amount of illicit drug detected on the MNA aptasensor was consistent with a sum of cocaine and BZE pharmacokinetics revealed by LC-MS in corresponding blood samples collected from mice (FIG. 14A). In humans, cocaine concentration in blood is about 0.06-0.1 mg/L or 0.06-0.1 μg/ml and our MNA aptasensor can detect cocaine as low as 1 ng/ml, far more sensitive than the detective level required in clinics. The ability of measuring illicit drug in real time effectively addresses any sample manipulation, a serious issue for drug screening onsite.

In brief, the MNA immunosensors and MNA aptasensors can measure proteins, DNA, or chemical analytes. The measurements can be real time or ex-vivo and this flexibility allows unlimited potentials for broad applications to diagnosis and monitor of a variety of diseases in clinics, pharmacy, at home, or bedside. A portable device is being engineered to assay analytes or biomarkers without the need of a laboratory as follows.

Design and fabrication of a small “all-in-one” prototype miniDia for assaying and acquiring signals on MNA-immunosensors

To assay the biomarkers at home or remotely, we designed and fabricated a small “all-in-one” prototype miniDia including an immunoassay station, a reagent-pre-filled microfluidic array, imaging acquisition chamber (Bottom, FIG. 15). It also includes MNA, and several detachable components (middle), and a self-sample collection kit for the user to collect blood sample from fingertip (upper, FIG. 15) or other body fluids. The miniDia can come as different versions dependent on the sample collection or data acquisition. We explain each part in detail in the following.

### Self-Sample Collection and Preparation

The miniDia can be used for detection of biomarkers from various types of samples. Sample collection includes, but are not limited to blood, oral and nasal swabs, urine, stool, etc. Herein, we take blood sample collection as an example. Since blood cells would impact the result, we need to remove blood cells during the sample collection and preparation.

Integration of a Plasma Separation Membrane into the Sample Chamber of the Reagents-Prefilled Microfluidic Array

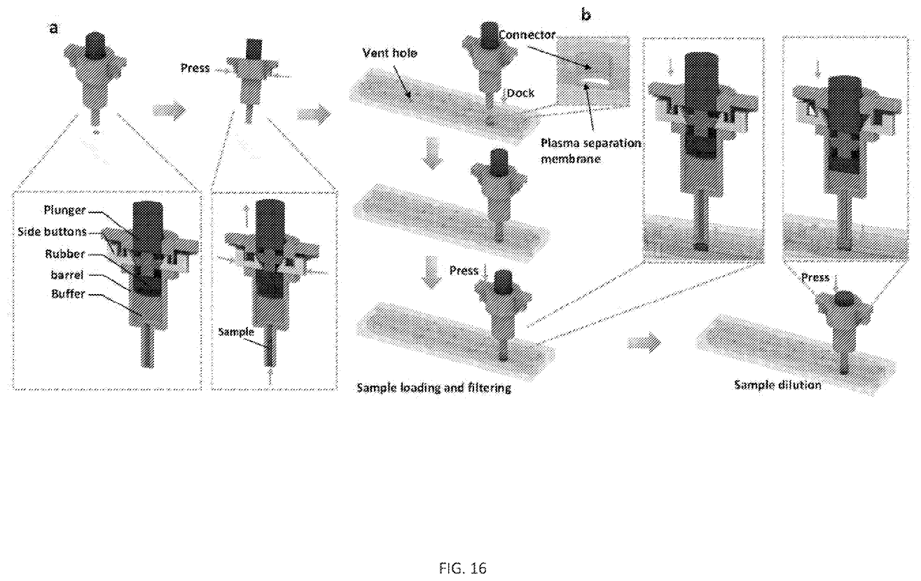

A self-blood sample collection kit contains a lancet to prick the finger and a blood collection/delivery accessory (FIG. 16a). The accessory has a capillary tube on the tip and a sample dilution buffer in the bottom of a syringe-like device that can suck the blood on the finger, mix it with the dilution buffer, and then deliver it to the sample chamber in the reagent-prefilled microfluidic array (FIG. 16b). The sample chamber has a plasma separation membrane to block the cells from entering the microfluidic array system. Due to high sensitivity of MNA-immunosensors, blood sample is usually diluted 50-200× depending on specific biomarkers or the disease.

### A Disk-Centrifuge for Sample Preparation

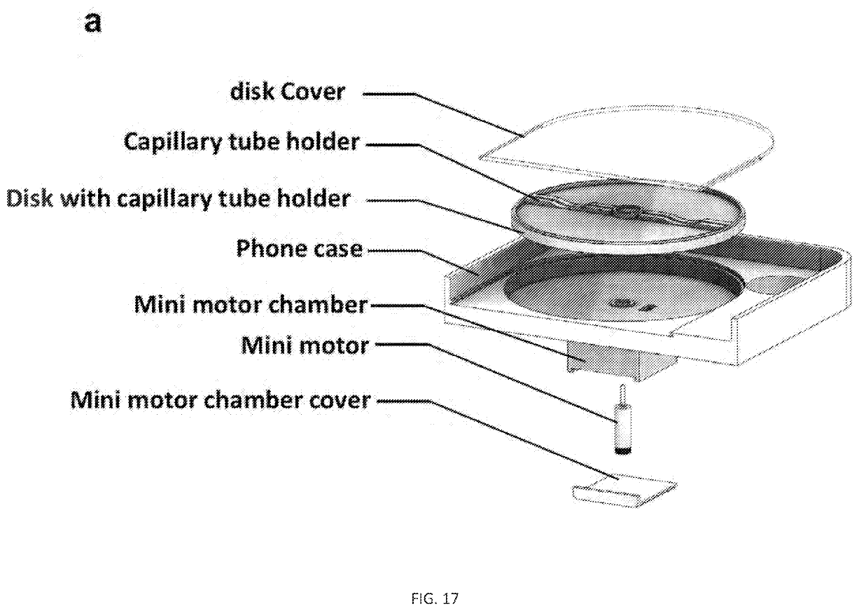

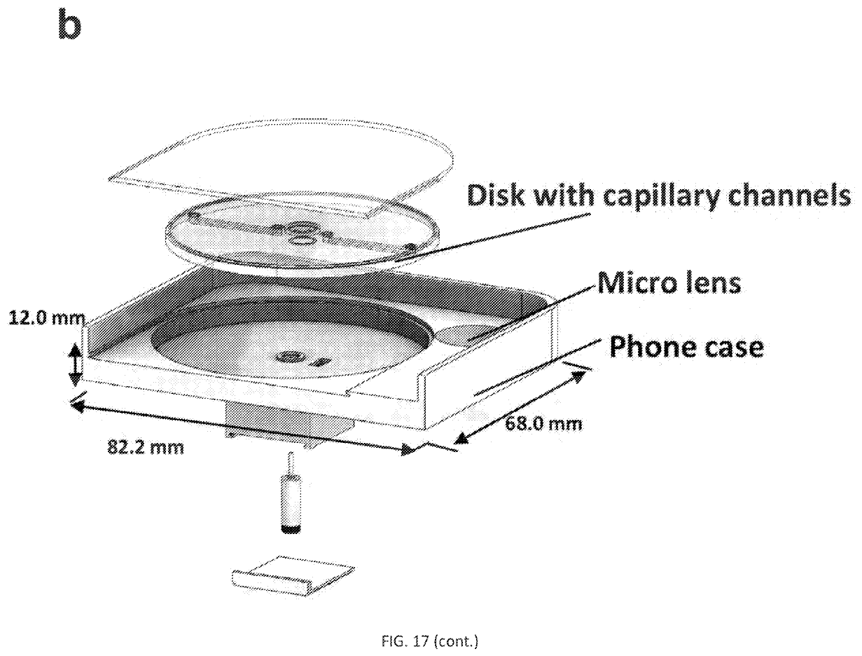

Alternatively, a disk-centrifuge is designed and fabricated as shown in FIG. 17. Two different disk-centrifuges are designed: a plastic disk holding a capillary tube (FIG. 17a) or have a capillary channel caved in the disk (FIG. 17b). The disk-centrifuge is integrated into the smartphone holder (Bottom, FIG. 16). A cost-effective mini coreless motor with a speed of 40,000 RPM (6 mm×12 mm) is installed in the mini motor chamber and operated completely by 3V battery. The disk is 58 mm in diameter and 1-2 mm in height and made with transparent material, so does its cover for easily seeing through. The size of phone case is 68.0×82.2×12.0 mm and can accommodate the disk perfectly. The disk can hold one or more capillary tubes or channels.

### A Disk-Centrifuge for Preparing Samples

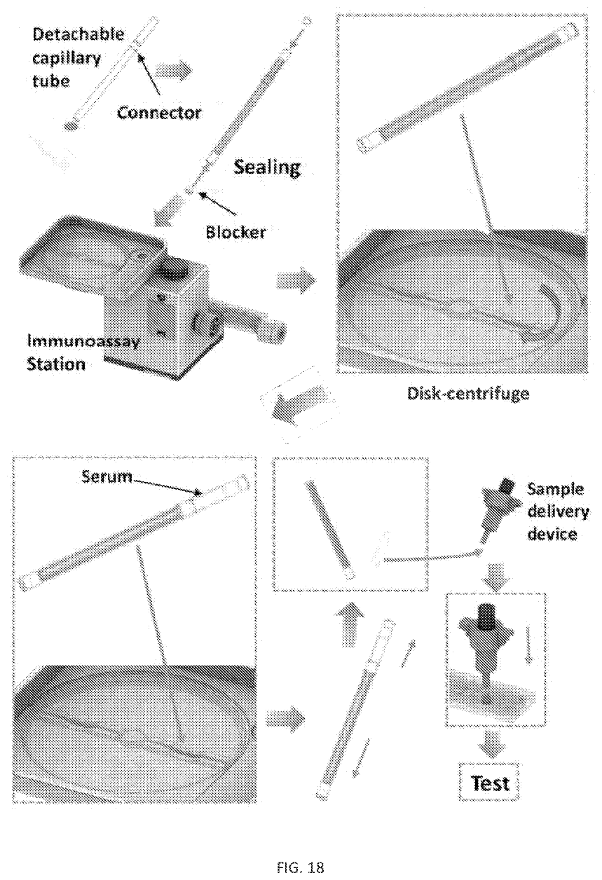

As shown in FIG. 18, a capillary tube can collect blood sample from pricked finger and sealed in both ends followed by inserting it into one of the two capillary tube holders in the disk for balancing. The disk cover is then placed to protect the sample prior to centrifugation. The design of the connector allows the user to separate the tube after centrifugation to obtain the plasma sample free of blood cells and then insert it into the sample delivery device to load the plasma sample into the microfluid array for the MNA assay.

A Disk-Centrifuge with a Capillary Channel for Sample Preparation

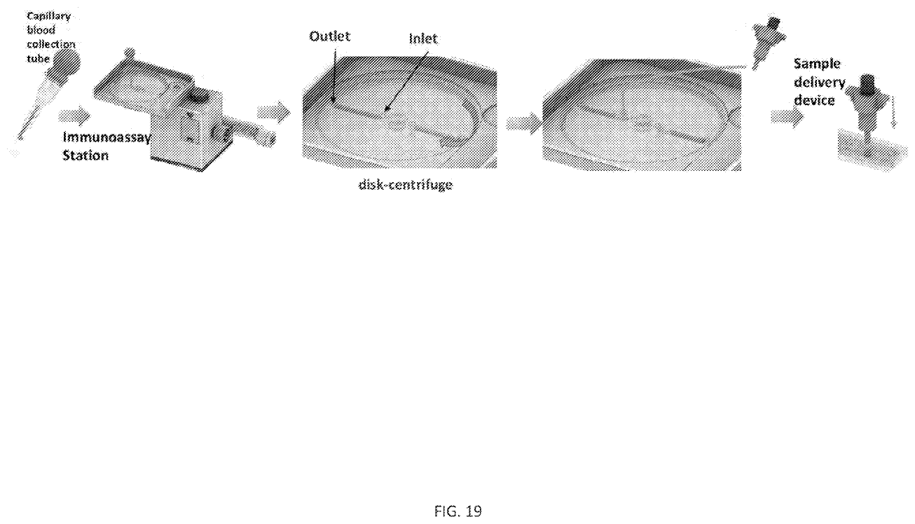

The capillary blood collection tube with a rubber bulb on top is used to absorb the blood sample and transfer the sample into the capillary channel via the inlet. The inlet and outlet are sealed to prevent sample leakage. In virtue of the interaction of forces generated by the disk rotation including centrifugal force, capillary force, Coriolis force, and Euler force, the blood cells are forced toward to the outlet part while plasma moving to the inlet part. The plasma can be collected by sample collection/delivery device from inlet. The plasma is loaded into the reagent-pre-filled microfluidic array. The disk is disposal for safety as it contains blood sample (FIG. 19).

Pre-filled reagent microfluidic array to deliver various agents and wash buffers into the MNA housing chamber sequentially.

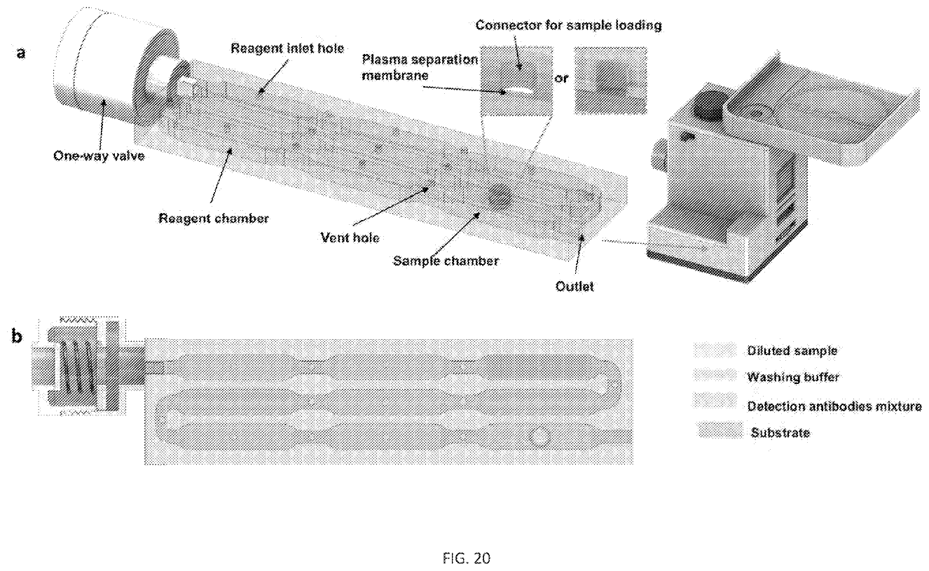

A reagent pre-filled microfluidic array with a dimension of 78×20×5 mm (height) is fabricated (FIG. 20). While microfluidic arrays have been used in different medical devices, this would be the first one engineering for immunoassay on MNA. The reagent-pre-filled microfluidic array includes 9 segments, one inlet port which is equipped with a one-way valve for air flow control, and one outlet port which is connected to immunoassay station (FIG. 20a). Dependent on the substrate in a specific MNA-immunosensor, the number of segments can be expanded to 15 or more. Reagent segments each are pre-filled sequentially with 180 pL via an inlet hole, and air is filled between two segments to eliminate intermixing of the reagents as illustrated (FIG. 20b). The volume of reagent is sufficient to cover the MNA. The first reagent is prefilled to the outlet end like plasma sample. The second and third reagent segments are filled with washing buffer, and then HRP-conjugated detection antibody, HRP-conjugated aptamer, washing buffer, substrate, and so on. It can be readily modified according to reagents required for a given immunosensor.

### Detachable Components

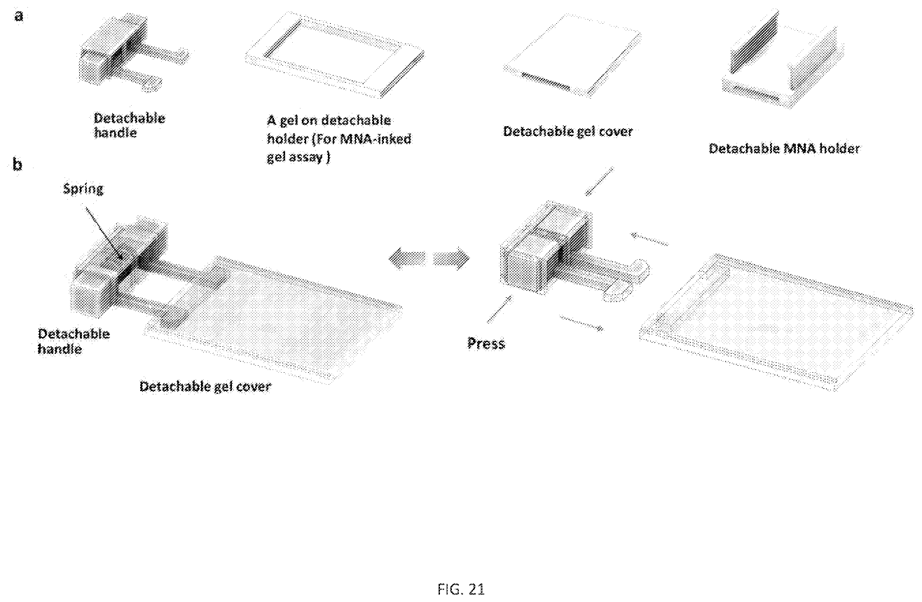

The detachable components include a handle, a gel holder, a gel cover, and an MNA holder for MNA and gel loading, gel staining, and imaging (FIG. 21a). The MNA and gel can be placed in a corresponding holder and inserted into the MiniDia device with a handle (FIG. 21b). We also include a gel cover to protect the gel from impact of washing and antibody reaction occurring in the immune reaction station beneath, offering more reliable results.

### MiniDia Design and Function

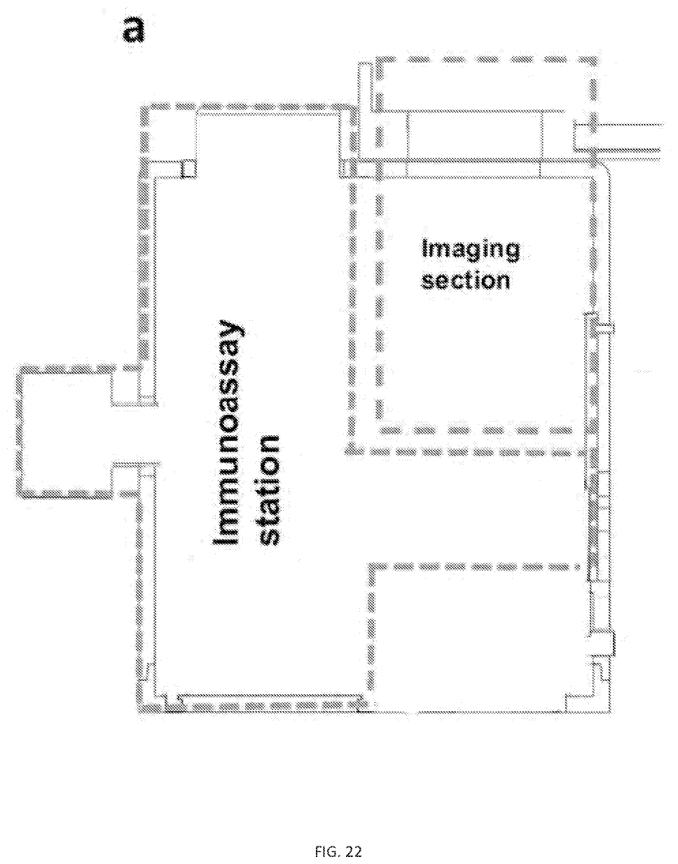

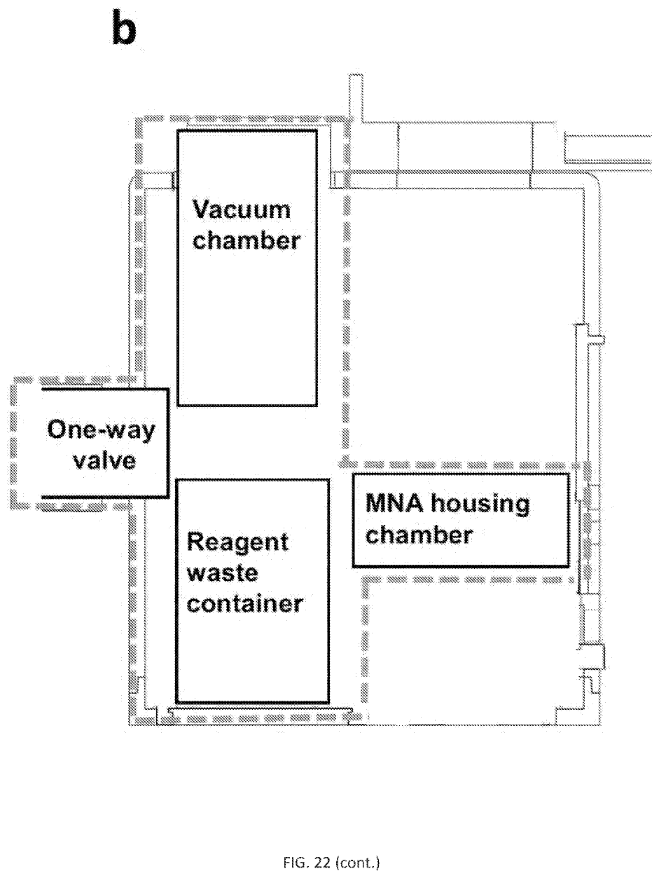

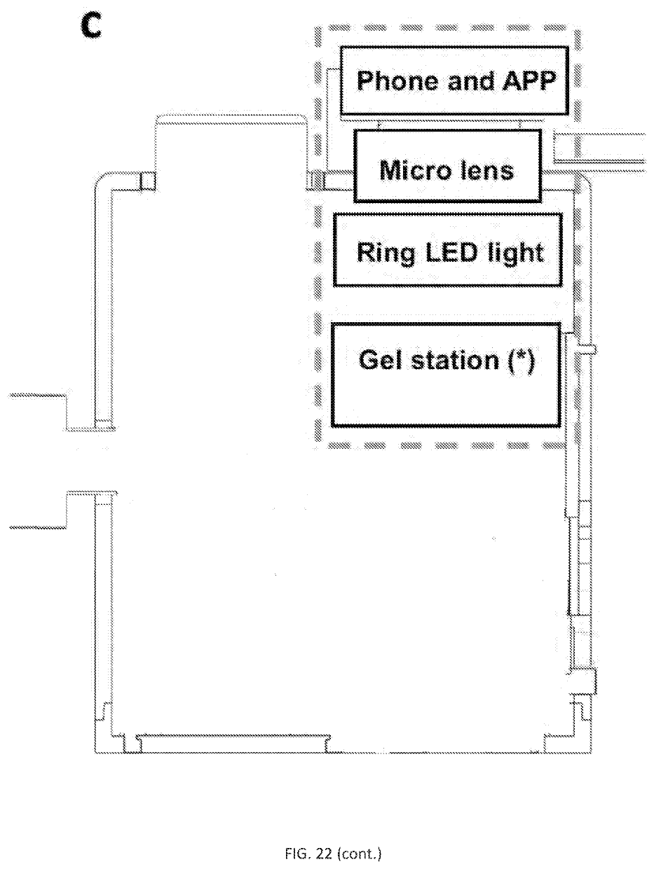

MiniDia includes two main parts: the imaging station to capture the signals on each MNA (red dash outline) and immunoassay station enabling the immunostaining on the surface of individual microneedles in the MNA (blue dash outline (FIG. 22a). The immunoassay station includes a vacuum chamber, a waste container, MNA housing chamber, and one way valve (FIG. 22b). Smartphone is required to take the imaging on top of the imaging station that holds microlens, ring LED light, and gel station (FIG. 22c). The MiniDia has a size of 67.5 (L)×59 (W)×69 (H) mm, just about a palm size.

### Immunoassay Operated by “On-Demand Vacuum”

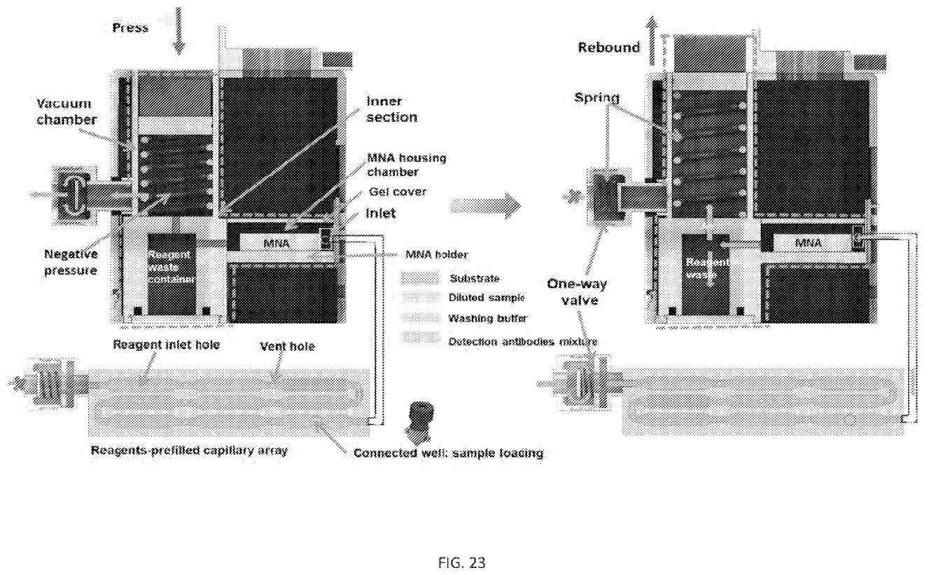

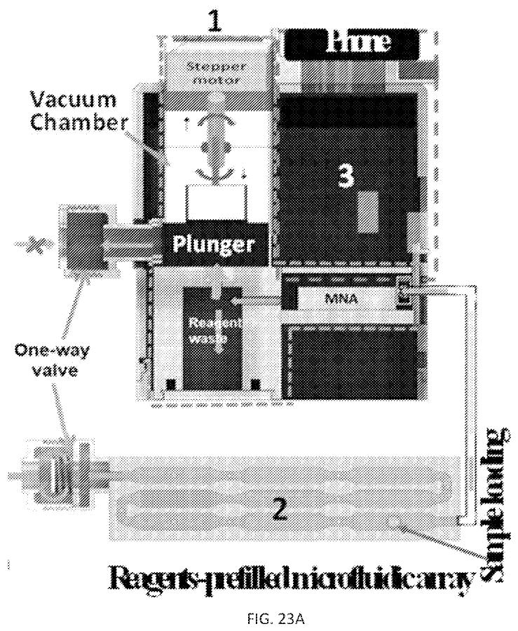

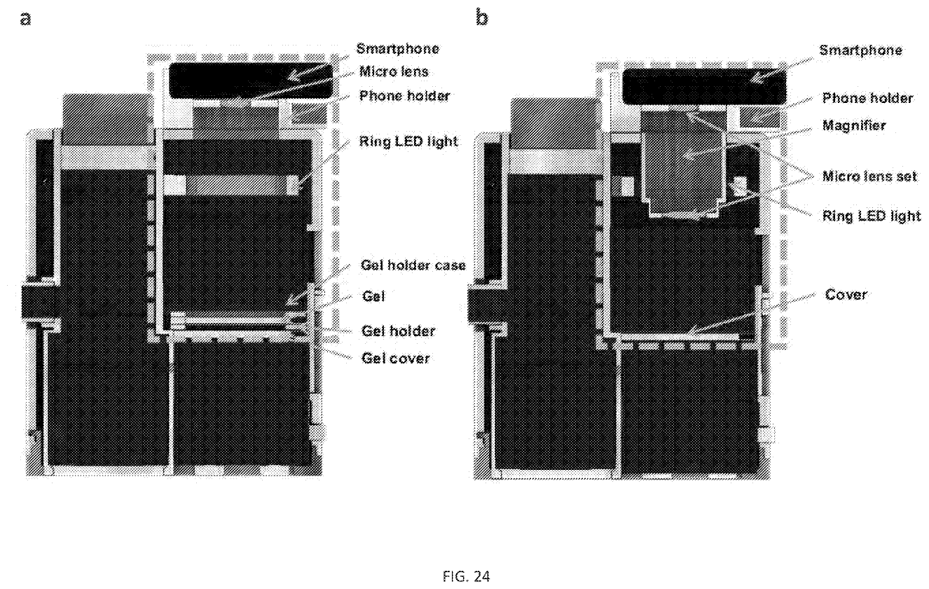

The MNA housing chamber is connected to the microfluidic array in one end and waste container in the other end (FIG. 23). It can accomplish sample loading, biomarker binding, washing, detection antibody binding, and washing after an MNA-immunosensor is inserted into the MNA chamber using a detachable MNA holder. To circumvent a power-consuming electrical pump, enable semi-automatic functionality, and reduce the cost and size of device, the design of “on-demand vacuum” allows a user mechanically to generate a negative pressure within the chamber to sequentially draw the pre-filled reagents in the microfluidic array into the immunoassay station (pink arrow, right). This vacuum chamber is constructed by a syringe-like chamber with a pressure button and a rubber circle on the button and spring and also connected to reagent waste container at the bottom. The principle of on-demand vacuum is as follows: a negative pressure is created in the chamber when the button is pressed squeezing all air out from immunoassay station, waste container, and the microfluidic array because the on-way valve only allows air out but not in (pink arrows, left). The negative pressure pulls the prefilled solutions in the microfluidic array into the immunoassay station sequentially, followed by drawing the solutions into the waste container (pink arrows, right). The volume of vacuum chamber is subject to the properties of the spring and reagent volume. The “on-demand vacuum” design requires an airtight sealed space, and the switch is designed to close the window to keep the airtight sealing condition of the entire immunoassay station outlined with blue dash line. The semi-automatic operation can be readily engineered to a fully automatic system for a more demanding site like bedside, clinics, pharmacy, battlefield using a time-controller to turn on and off the pressure button.

In another embodiment, the immunoassay station may be operated by a stepper motor that is controlled by a stepper motor driver to sequentially draw the pre-filled reagents in the microfluidic array into the immunoassay station (FIG. 23A, pink arrows). The stepper motor operation is accomplished by a plunger, a stepper motor, a stepper motor driver, an Arduino board, and a switch button. A 12V power supply can provide the required voltage for the stepper motor driver and Arduino board. The Arduino will control the entire process of the system. A switch button will start the process by lowering the plunger to squeeze the air out of the vacuum chamber, but not allowing air in using a one-way valve. The stepper motor driver will be adjusted for its speed, and the force of the motor will be transferred from a torque to a linear force. The timing specification values will be provided with the commands given by Arduino. The stepper motor rotates in a reverse way to rebound the plunger to its early position (up) to create negative pressure and draw the reagent into the MNA housing chamber from the microfluidic array.

### Imaging Station

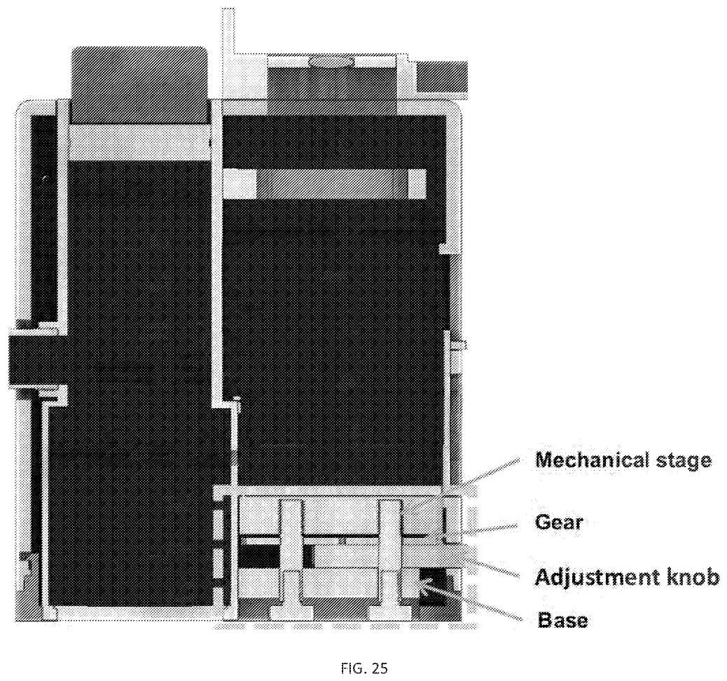

FIG. 24 shows the component of imaging section. The steps of gel staining and imaging are accomplished in the section. The imaging station contains a micro lens (FIG. 24a) or magnifier (FIG. 24b) to amplify the gel or microneedle images, respectively. In addition, a ring of multiple emitting diodes (LEDs) is placed encircling the entire chamber wall to obtain uniform illumination of the gel or microneedles in all dimensions. A mechanical stage beneath the MNA housing chamber can lift the MNA and penetrate the substrate-saturated gel prior to imaging (FIG. 25). The LEDs are operated by the same battery power used for the disk-centrifuge, but they are not competitive because they are used in different times. An optimized distance between the stained gel and micro-lens is pre-justified to ensure perfect focus.

### MNA-Inked Gel Assay

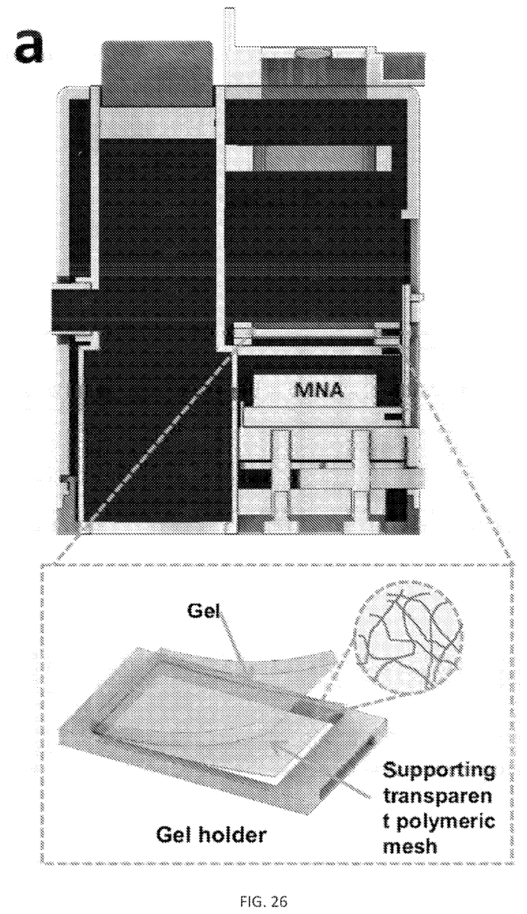

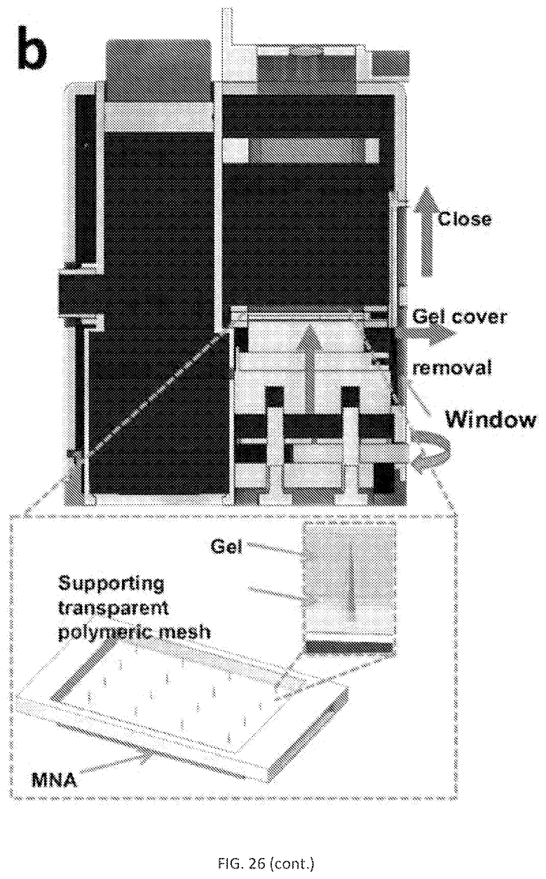

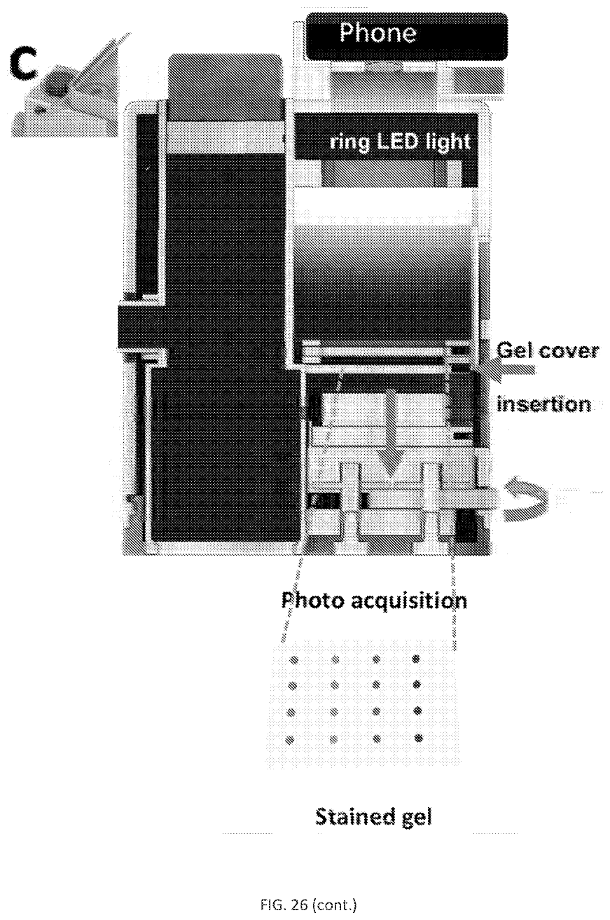

For MNA-inked gel assay, the imaging section also includes a gel holder case, a detachable gel holder, and a substrate-saturated gel (FIG. 24b). A gel holder case consists of a polymeric mesh to support the substrate-saturated gel and a handle (FIG. 26a, lower panel). The polymeric mesh can include, but is not limited to polypropylene (PP), polyethylene (PE), nylon etc. The transparent gel can be made of natural polymer such as gelatin, alginate, chitosan, collagen, elastin, fibrin, hyaluronic acid, silk fibroin, their derivatives, biocompatible synthetic polymer and/or co-polymer, biocompatible synthetic polymer/natural polymer composites, and the like. The substrates can be enzyme substrates or fluorescent amplifiers such as plasmonic nanostructures, and so on, aiming at great enhancement of the binding signals. The gel holder is inserted to the gel holder case in the miniDia and protected from the possible impact of washing and biomarker binding during the immunoassay by a gel cover placed beneath. After immunoassay in the MNA housing chamber, the gel cover is pulled out and the stained MNA is lifted to insert into the gel for 15 min (FIG. 26b), after which the LED light turns on for imaging with smartphone (FIG. 24b and FIG. 2c). Because MNA-inked signals in the 2D gel can be conveniently and efficiently imaged (FIG. 26c), a simple micro lens is sufficient to aid smartphone camera for gel imaging.

### Direct Imaging of Individual Microneedles in an MNA-Immunosensor

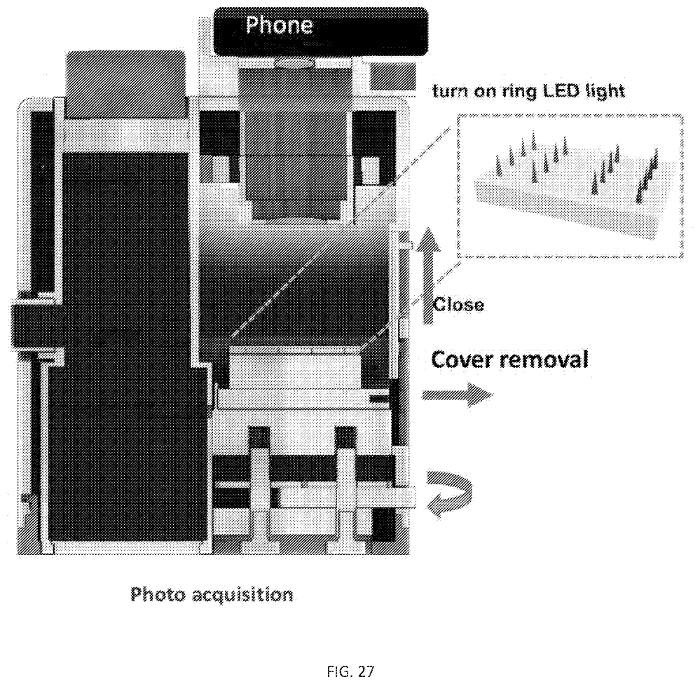

Slightly different from the MNA-inked assay described above, direct imaging of individual microneedles in the MNA by smartphone doesn't involve a gel in the imaging station. Due to the 3D structure of the microneedles, the image of each microneedle is preferably captured one-by-one in the array. A high magnifier is required to achieve a clear image of a stained microneedle with a high quality or an array of micro-lenses with high magnification may also work (FIG. 24c and FIG. 27).

### Smartphone App

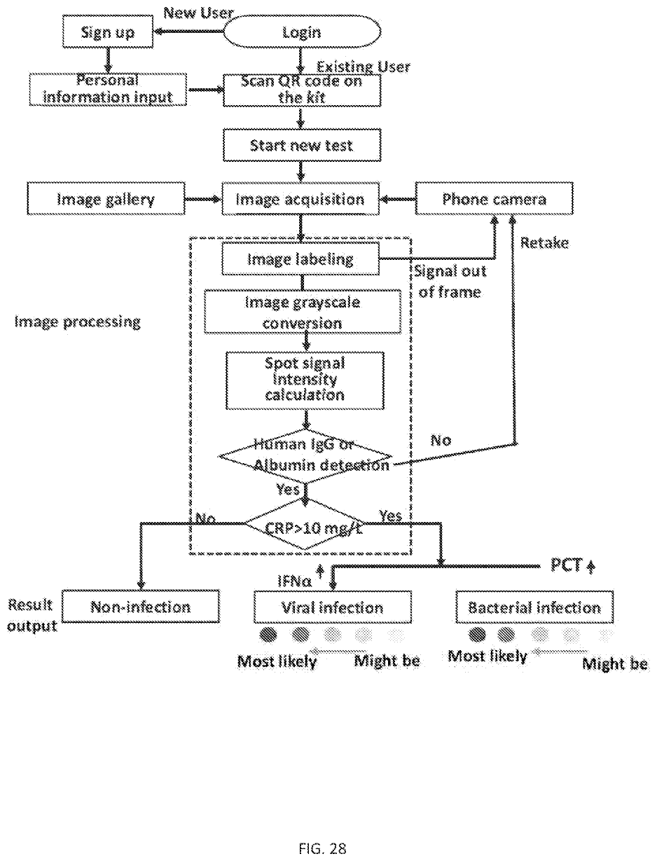

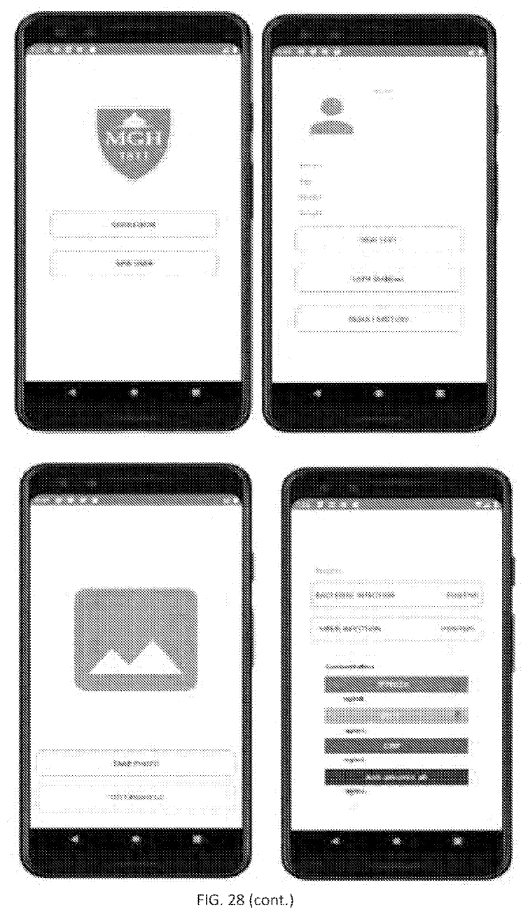

We employed Android studio (4.0.1) to develop an App using Kotlin as coding language. The virtual device is Google Pixel 3 (API 30), and the system is Android 10.0+. As shown in FIG. 28, we build a user-interface-friendly and easy-to-use app, which also keeps user's personal information private.

A user can create an account by inputting some basic information including name, gender, age, weight, email, and password. If the user has a doctor, the user can input his doctor's contact information. Since different MNA-immunosensors can measure different panels of biomarkers for different disorders, the user can scan the QR code on the back of the reagent-pre-filled microfluidic array of the kit and download the specific data package associated with a specific App following the step-by-step pictorial instructions.

After capturing an image or a photo with high resolution via the smartphone, the App can convert the color mode to “Grayscale” and quantify the intensity of the images. In the background, we generate an array of circles aligning with the corresponding biomarkers in a specific MNA-immunosensor. The intensity of each spot in the circle is quantified to determine a concentration of the biomarker by a linear equation of the corresponding biomarker that is programmed in the App. The result is reported as “positive” or “negative” based on the concentrations of these biomarkers either higher or lower than a preset cutoff value for each biomarker, respectively. It may also display “Might be” to “most likely” in the event that the imaging pattern is not well defined, which can be further modified and improved based on the clinical studies or with additional biomarkers integrated in the immunosensor. Moreover, the result can be uploaded and stored in commercial Cloud storage or directly sent to the user's doctor for analysis and diagnosis. As the time goes, clinical data can be collected in a vast number to aid diagnosis more precisely. Such data are critically lacking today and urgently needed for future eCare and digital health system.

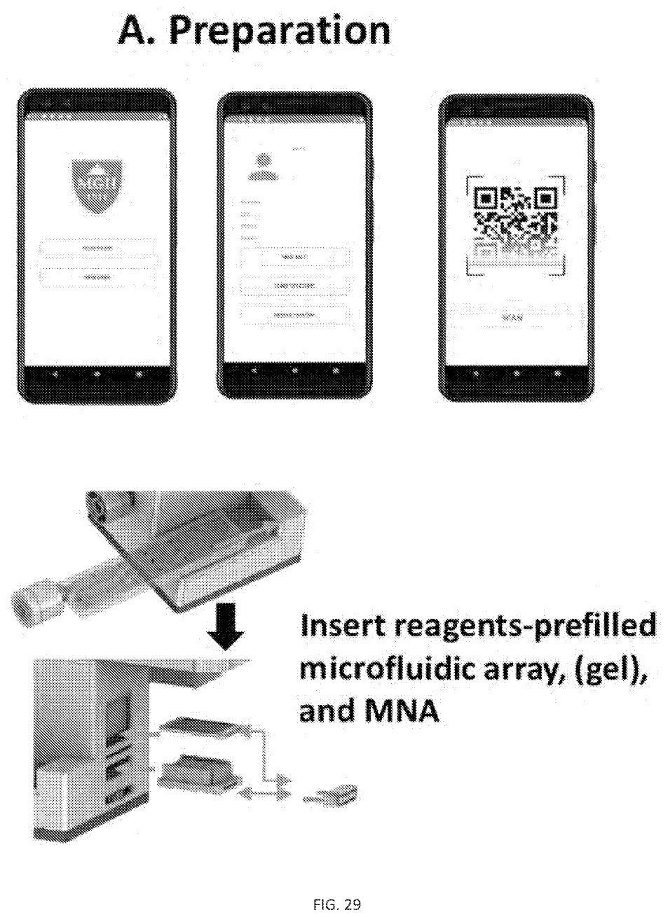

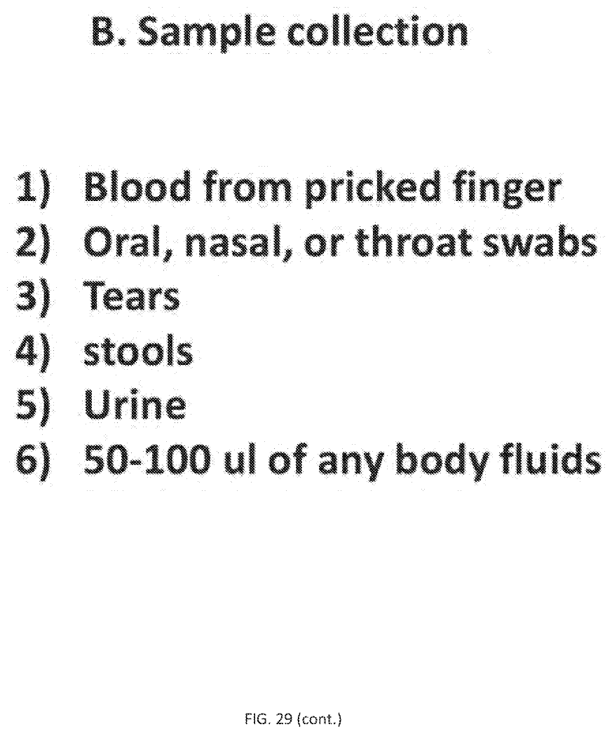

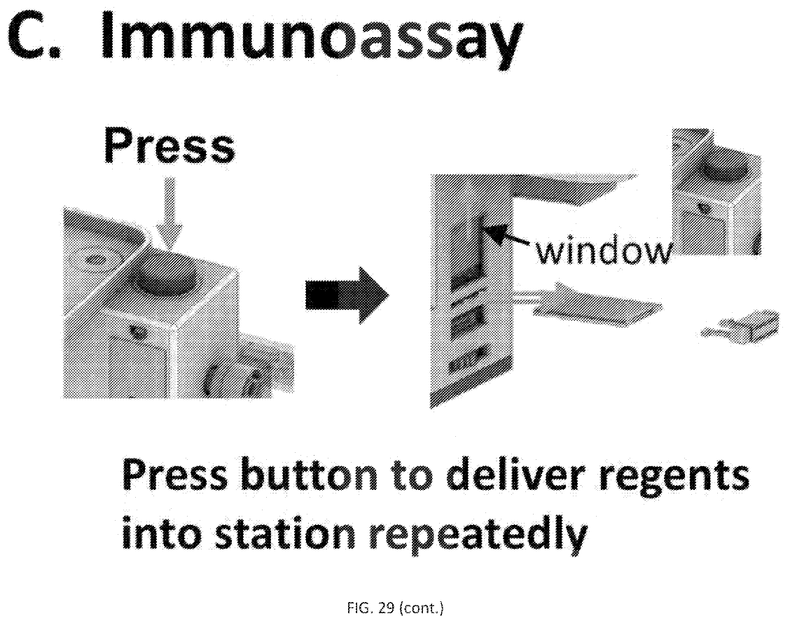

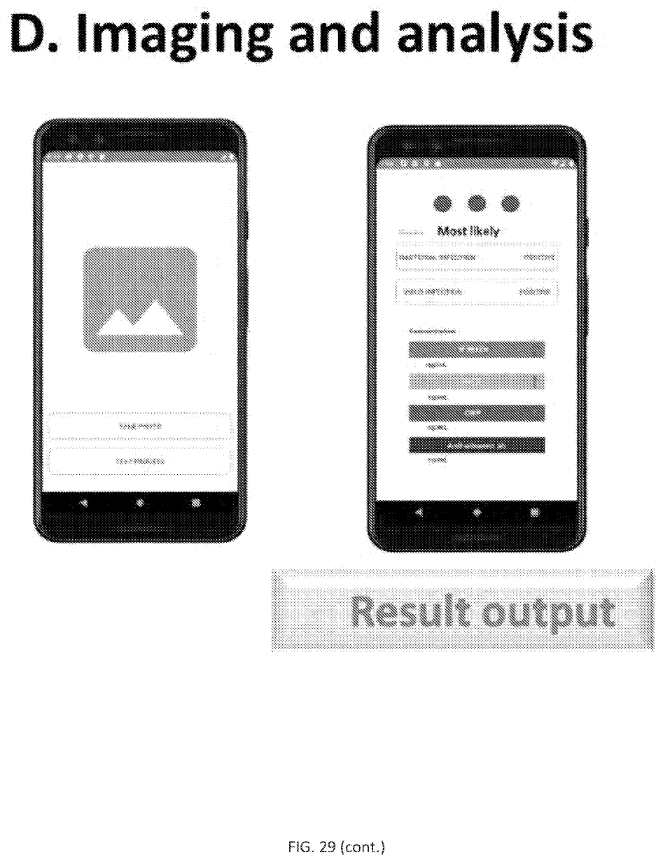

In summary, a user can download, register, and login into the APP, followed by scanning the QR code printed on the back of a specific reagents-prefilled microfluidic array (FIG. 29A, upper). A specific MNA and gel are inserted into the miniDia and reagents-prefilled microfluidic array is connected to the immunoassay station (FIG. 29A, low panel). After the preparation, the user has three different ways to collect and transfer the blood sample into the sample chamber of the microfluidic array (FIG. 29B). In addition to blood, any fluid samples can be processed similarly and loaded into the sample chamber (FIG. 29B). The immunoassay is processed by pressing the button of MiniDia several times following the introduction on the APP (FIG. 29C). Finally, the user takes photo with smartphone and the APP would analyze and show the result instantly or send the data to his/her physicians for comparison with his/her previous data for diagnosis and treatment decision (FIG. 29D). Alternatively, the data can be sent to the cloud database worldwide, especially at the event of pandemics.

A Portable Device for Real Time Measurement of Biomarkers with MNA-Based Sensors

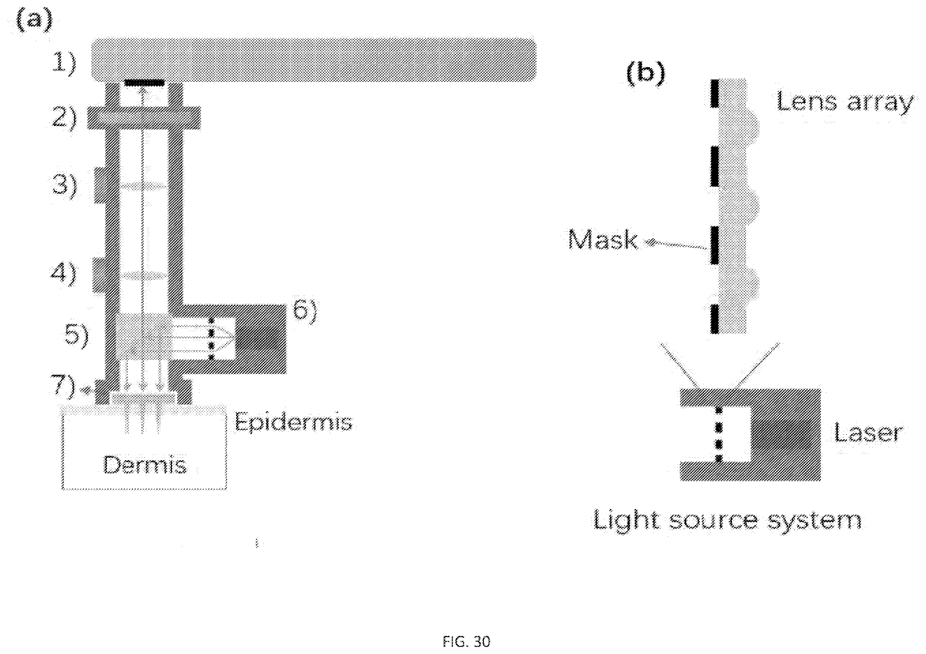

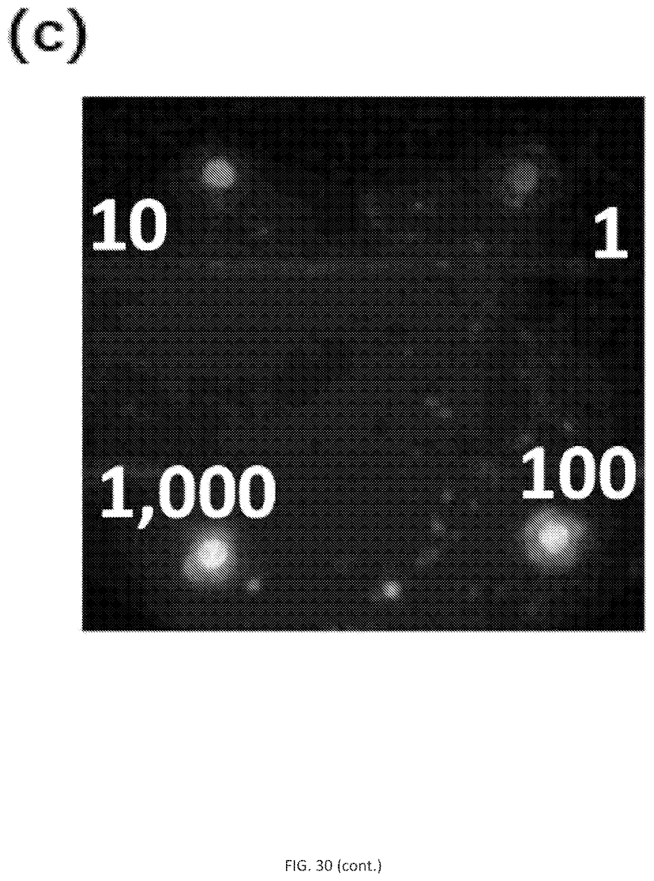

A portable prototype is engineered for MNA-aptasensor to measure analytes in real time. The portable prototype is depicted in FIG. 30 and includes 1) CCD camera connected with smartphone; 2) long-pass filter; 3) zoom lens; 4) objective lens; 5) cube beam splitter; 6) multi-beams source system; 7) MNA-aptasensors that are wearable and direct light into the upper dermis. The light source system consists of a microlens array that is placed in the front of a laser source (FIG. 30b). The microlens array is covered with a mask to provide multi-beams of the lasers. A representative fluorescent photograph of the four microneedles, excited under 532 nm laser, was captured by a smartphone. The four microneedles were modified by Cy3.5-labeled cocaine-specific aptamer at a concentration of 1, 10, 100, or 1,000 nM.

### Multi-Beam Laser

A semiconductor laser unit can be used and integrated to the device due to their low cost and small size. The emitting light from laser source is collimated by a low-cost plastic micro-lens array which is covered by a mask (FIG. 30b). The purpose of the mask with circular hole array is to only allow the collimated light passing through, so that the total power projected on the skin is reduced. By integrating the mask and the microlens array, the laser beam array can be obtained. A 3×3 microlens array was fabricated by using polymethyl methacrylate (PMMA), a material with excellent transparency. The PMMA solution was casted on a poly(dimethyl siloxane) (PDMS) mold with a negative pattern of the microlens. After removing the bubble in PMMA solution by centrifugation, the mold was heated on a hot plate at 85° C. for 24 h. The solidified PMMA microlens array was then exfoliated from the mold. The laser light with a central wavelength of 532 nm was collimated by the microlens, and projects onto the microneedle base. Considering the size of 3×3 microneedle (36 mm² area), a compact laser unit with a power up to 150 mW was used to provide each microneedle with a beam of 0.5 mm diameter and 0.9 W/cm² light intensity, which was required to induce leakage of blood vessels within the dermis and obtain bright fluorescent of the microneedles for CCD imaging.

### Optical Excitation and Collection System

To excite Cy3.5 and collect the fluorescence, a cube beam splitter was used to reflect the laser beams onto the base of the microneedle patch and, in the meantime, allow the excited fluorescent to be collected by the magnifying adapter (FIG. 30a). To clearly capture the fluorescence from individual microneedles, a long-pass filter was placed between the microneedles and the adapter to remove the excitation laser while allowing the fluorescent passing through. The adapter mounted on the mobile phone consisted of objective lens and magnifying lens, offering a magnification in the range of 60-100×. The objective lens in the adapter was to collect light from microneedles to form real image of the luminescent microneedles, and the right lens magnified the image so that the camera of mobile could take a clear magnified image of the microneedles. An APP for the mobile phone can be programed to image and quantify the color intensities on each microneedle. FIG. 30c shows the top-view of the Cy3.5 decorated microneedles under laser irradiation. This image was taken by Smartphone with a magnifying adapter and a filter. The image clearly demonstrates different fluorescence intensities among the four microneedles, corresponding to the concentrations of aptamer immobilized on the individual microneedles. The results demonstrate the feasibility of the optical diagnosis system.

The portable device can be engineered for real time diagnosis and monitor of a variety of disorders. The microneedles in the array can be conjugated with a specific aptamer in a one aptamer-one microneedle fashion and multiple analytes like a panel of illicit drugs can be measured in a finger in real time. Alternatively, the microneedles in the array can be illuminated individually or as a panel (like 3-4 together) at different time points like every 2, 4, 6, or 10 hrs to measure several analytes continuously via desirable time points of individual microneedles. The biomarker or analyte extravasation would take place only if the laser is administered via the targeted microneedle.

### Sample Free MNA Immunoassay

The optical MNA platform makes it possible to detect single or multi-biomarkers in a sample free manner. Specifically, we can insert the transparent, MNA-immunosensor directly into the skin and illuminate the skin with green light through the MNA to induce biomarker extravasation from skin capillary so that the capturing antibodies on the MNA can bind to the specific biomarkers leaked from the circulation. Then, the MNA is removed and inserted into the MNA housing chamber in the miniDia for immunostaining as detailed above (FIGS. 23-28). By a combination of an optically transparent MNA-immunosensors or MNA-aptasensors, multiplex detection of circulating biomarkers can be conducted miniDia without drawing any blood.

### EXAMPLES

The following provide non-limiting examples of embodiments of the disclosure:

Long-term disease activity monitoring at home is of importance and highly demanded for many diseases. Since diseases usually involve complicated biological activities and cannot be determined by merely one biomarker, multiplex biomarkers measurement has shown considerable promise in improvement of disease diagnosis accuracy. However, multiplex biomarkers detection at point-of-care (PoC) remains a great challenge. Herein, we develop a microneedle array (MNA) based assay capable of simultaneously qualifying a panel of biomarkers on a single microneedle in one-microneedle-one biomarker fashion. Upon embedment of MNA—“ink” in a gel saturated with color substrate, this assay, which we named MNA gel assay, enables transfer of colorimetric signals from a 3D microneedle to a 2D gel where the immunoassay sandwich formation on MN catalyzes color substrate in gel, inducing the accumulation of the blue oxidation product surrounding the sites of MNs to mimic “inking” process of printing for convenient, precise and efficient acquisition of signals at PoC. To demonstrate the proof of concept of this assay in multi-biomarkers quantification to improve disease diagnosis accuracy, five biomarkers were selected for systemic lupus erythematosus (SLE) diagnosis because antinuclear antibodies (ANA) test, the current commercial lab blood test for SLE diagnosis (2019 European League Against Rheumatism/American College of Rheumatology Classification Criteria for SLE), has a very poor specificity (57%). The MNAs exhibited high sensitivity and selectivity, and the limit of detection (LoD) of the assays were at least 10-fold better than the commercial enzyme linked immunosorbent assay (ELISA). The MNAs and corresponding ELISA kits are used to conduct side-by-side measurement of five serum biomarkers in SLE patients (n=42) and healthy controls (n=34). The results obtained by MNAs were well validated by traditional ELISA kits and the selected biomarkers panel can effectively discriminate lupus patients from healthy controls. In particular, the specificity of our assay for detection of a selected biomarker panel was 97%, representing 70% improvement in comparison with ANA test, and the sensitivity of our assay was competitive with that of ANA test. These results demonstrate that MNAs provide a platform with ability for accurate measurement of multiplex biomarkers that can accommodate as many biomarkers as needed, which has promising potential for disease diagnosis, health monitoring and tracking progress in treatment at PoC.

In the past two decades, point-of-care (PoC) technologies for measuring blood glucose in management of diabetes, urine chorionic gonadotropin (HCG) in pregnancy testing, and recently COVID-19 viral spike protein in COVID rapid tests demonstrate a great success and benefit millions of patients. However, these tests are limited by only one biomarker measurement, and the technologies are difficult to extend to other diseases or healthcare needs. Scientists have devoted enormous efforts to replicate the success of these home-based tests for measuring multi-biomarkers in the past two decades. But, as of today, there are no widely used commercial point-of-care (PoC) diagnostic tools that can simultaneously measure a panel of biomarkers remotely without a laboratory. While the golden standard enzyme-linked immunoassay (ELISA) is a routine assay for multi-biomarker detection in clinics, the assay depends on sophisticated instruments and trained technicians in fully equipped laboratories. Other technologies capable of detecting multi-biomarkers, such as protein microarray, rely on expensive equipment and extensively trained staffs and hardly become a lab-free routine.

Microneedle array (MNA) or micro-projector array (MPA) which has numerous microneedles or micro-projectors with various shapes in 3D structure arranging in arrays on a base, allows one microneedle or micro-projector to be immobilized with one specific capture element such as antibody, ligand, antigen, or aptamer covalently, enabling detection of multiplex biomarkers. Given a large surface area of microneedles, more capture elements can be immobilized, resulting in significant improvement of sensitivity, compared to protein microarray with a flat surface of a glass slide. Colorimetric assay via imaging and analysis by smartphone is a popular approach for PoC test, yet it is difficult to achieve the same perspective of directly stained microneedles or projectors in one image due to the space perspective of microneedles or projectors arrangement and 3D structure of microneedles or projectors, resulting in inaccurate quantification of biomarkers concentration. Since MNA with cone-shaped microneedles can insert gel with minimization of gel damage, and the catalyzed colorimetric substrate can accumulate on the site of a gel where the microneedles insert, MNA was used in this study. Last decade has witnessed that MNA has attracted increasing interesting in building various MNA-based biosensors because of its unique promising properties.

Although there have been considerable efforts in MNA-based biosensors, most of previous reported MNAs are only single analyte detection because the micron size of microneedle makes it difficult to individual MNA surface modification for multi-biomarker detection, and performance of MNA is only investigated in animal model studies with intravenously injection of target molecule without clinical samples or on-body studies. There are only a few emerging MNAs for multiplex biomarkers detection, but further improvement is required to address their clear limitations. Furthermore, all of these seniors rely on expensive instruments for signal detection such as confocal or fluorescent microscopy, which makes them difficult to convert to PoC applications. Recently, a wearable microneedle array that was used for real-time monitoring of multiple metabolites was reported and on-body studied was carried out, but it is limited without investigation of a panel of protein biomarkers detection and its application in a disease model. There is an increasing demand to develop a method that can acquire signals conveniently, precisely and efficiently from for MNA assays with ability to detect multi-biomarkers at home.

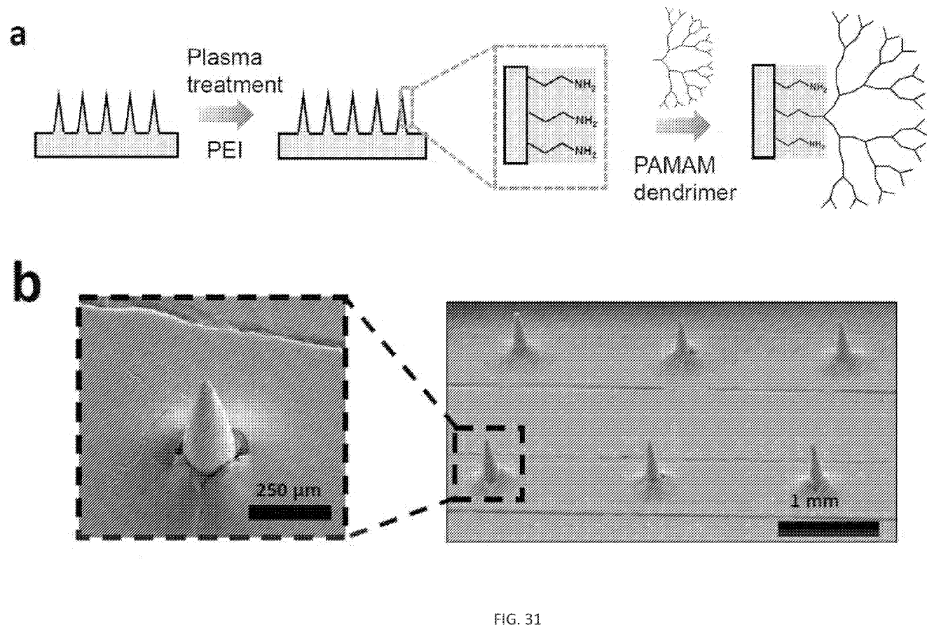

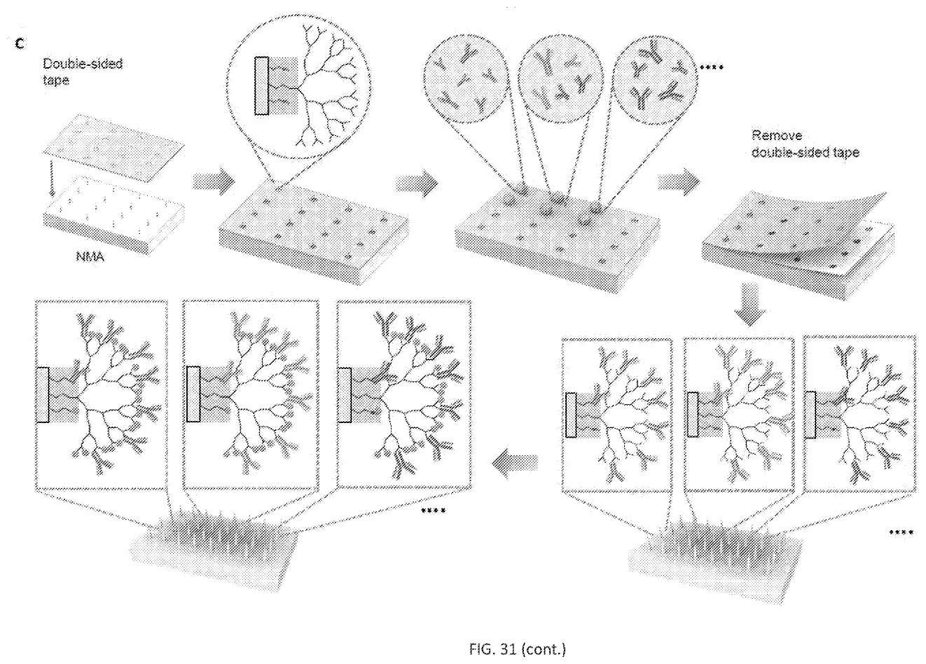

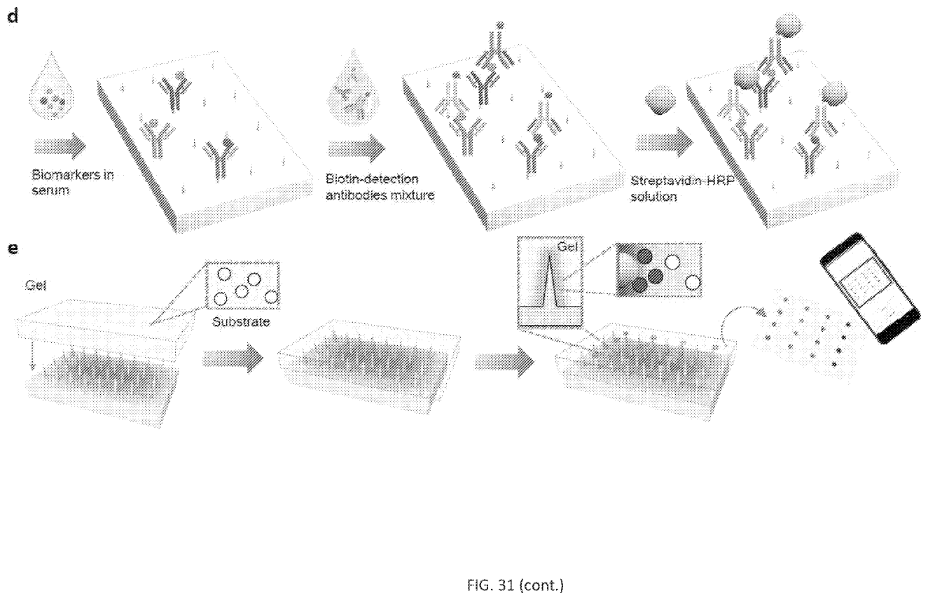

As shown in FIG. 31, inspired by ancient printing technologies, an extraordinary technological revolution in the history of printing, we developed a unique colorimetric assay, this study describes for the first time to develop an MNA gel assay for multi-biomarkers detection based on immunoassay sandwich formation through a flexible and efficient signal transfer from a 3D microneedle to a 2D gel, facilitating image acquisition and analysis by a smartphone in place of expensive laboratory instruments for PoC diagnosis and health monitoring. As movable type printing has the matrix consisted of molds used to cast letters which can be arranged flexibly, the different microneedles on MNA can be modified with a different panel of capture elements to expand to the diagnosis of many different diseases. MNA with surface modification containing abundant amount of amine groups enables covalent immobilization of capture antibodies, and sandwich immunoassay can be established after capturing the biomarkers in the patient's serum samples, followed by the addition of detection antibodies. The horseradish peroxidase (HRP) of a sandwich immune complex on MNA—the “matrix” can catalyze colorimetric substrate—the “ink”—on paper, thereby the colorimetric signal can be “stamped” in a gel, resulting in color spots with different intensity. Therefore, we can measure the biomarker by analyzing the intensity of color spots. In this study, we fabricated and characterized developed MNA, and investigated the performance of this assay including sensitivity, specificity and LoD to detect biomarkers (from high abundance to low abundance biomarkers). To demonstrate its clinical application potential to address the problem that existing blood test with low specificity, we carried out our assay to measure 5 selected biomarkers either single or combinations in serum samples from SLE patients (n=42) and healthy controls (n=34) and utilized ELISA to validate the results from this assay.

### Materials and Methods

### Materials

Poly(methyl methacrylate) (PMMA) (Mw˜120000), poly(ethyleneimine) (PEI) (Mn˜60000; Mw˜750000), ethyl acetate, PAMAM dendrimer (ethylenediamine core, generation 0, 4.0, 5.0, and 6.0 solution), 1-Ethyl-3-(3-dimethylaminopropyl)carbodiimide (EDC), N-hydroxysuccinimide (NHS), suberic acid bis(3-sulfo-N-hydroxysuccinimide ester), sodium hydroxide (NaOH), isopropanol, double-stranded DNA (dsDNA), albumin methylated from bovine serum (mBSA), and bovine serum albumin (BSA) were purchased from Sigma-Aldrich (Waltham, MA, USA). SeramunBlau spot dark 3,3′,5,5′-Tetramethylbenzidine (TMB) substrate was obtained from Seramun Diagnostica GmbH (Heidesee, Germany). Biotin-Goat Anti-Human IgG was purchased from Jackson ImmunoResearch (West Grove, PA, USA). Streptavidin-horseradish peroxidase (HRP) was purchased from Abcam (Waltham, MA, USA). Polydimethylsiloxane silicone (PDMS) elastomers base and curing agent (SYLGAR 184 Silicone Elastomer Kit) were obtained from Dow (Midland, MI, USA). PBS was purchased from Life Technologies (Carlsbad, CA, USA). Human C-reactive protein (CRP) (DY1707), Human insulin-like growth factor binding protein-2 (IGFBP-2) (DY674), Human sTNF RII/TNFRSF lB (DY726), and Human VCAM-1/CD106 DuoSet ELISA kits were purchased from R&D System (DY809) (R&D Systems, Minneapolis, MN, USA). Distilled water was obtained by using a Millipore Milli-Q ultrapure water purification system (Burlington, MA, USA).

### Clinical Samples

Serum samples from lupus patients (N=42) and healthy controls (N=34) were provided by the University of Houston, Houston, Texas, USA. The samples were collected under an institutional approved IRB protocol. All samples were aliquoted and stored at −80° C. until use.

### Fabrication of PDMS-MNA Mold

An MNA was fabricated according to previous study with modification. Briefly, an original MNA mold fabricated in our previous study was used for fabrication of PDMS-MNA mold. PDMS elastomer base solution mixed with curing agent at a 10:1 ratio was poured into a well of a 6 well plate, and mixed well, followed by centrifuge at 2,000 rpm for 10 min to remove bubbles. The original MNA mold was then placed into the mixture, and bubbles were removed under vacuum. The mixture was then heated at 85° C. for 3 hours. The original MNA mold was removed after cooling to obtain a female PDMS-MNA mold. 1 mL of PMMA solution was added into the female PDMS-MNA mold, followed by centrifugation at 4,000 rpm for 15 min.

### Fabrication of MNA and Surface Modifications

The PMMA solution (20% w/v) was prepared by dissolving at ethyl acetate and stirring at 78° C. for overnight prior to its addition to the female PDMS-MNA mold. The casting process occurred at 80° C. for 4 h to remove ethyl acetate, followed by adding 1 mL of PMMA solution to cover the first layer of dried PMMA at 85° C. overnight. The process was repeated once.

After PMMA was dried and cooled, the resultant MNA was carefully peeled from the female PDMS-MNA mold and was washed with 2-propanol for three times, followed by air-dry and oxygen plasma treatment with a Technics 500-II Plasma etcher (300 W) for 3 min. The plasma treated MNA was then immediately immersed in PEI solution (10% v/v, pH 11) at 60° C. and stirring for 6 hours. PAMAM dendrimers with ethylenediamine core (2 μM) were conjugated onto the PEI-modified surface of each microneedle in the MNA through using suberic acid bis(3-sulfo-N-hydroxysuccinimide ester) sodium salt (2 μM) as a cross-linking reagent.

### Immobilization of Multiplex Capture Elements on a Single MNA in One Capture Element on One MN Fashion

We use a laser to punch an array of tiny holes precisely aligning with the microneedles in the MNA on a double-side tape with a hydrophobic surface. The tape was then used to covered MNA base, and each hole formed by the tape and MNA base surface served as microcontainer for the specific capture antibody immobilization on the corresponding microneedles on MNA. Next, EDC and NHS were dissolved in MES buffer (10 nM), followed by addition of capture antibody. The mixture solution then was filled into to targeted coupling reaction microcontainer to covalently conjugate it onto the targeted microneedles via EDC/NHS coupling reaction, allowing reaction for 5 hours. After reaction was done, the tape was removed carefully. A washing buffer (PBS contained 0.05% Tween-20) was used to wash the MNA every 5 min for a total of three times to remove unreacted reagents. Finally, non-specific binding on the MNA was blocked by 2% skimmed milk at 36° C. for 1 hour and then washing every 5 min for a total of three times. For the detection of anti-ds-DNA antibody, the targeted microneedles were incubated mBSA solution, followed by addition of ds-DNA solution. The steps of washing and blocking non-specific binding were similar to that was described above.

### MNA Gel Assay Evaluations

To evaluate the sensitivity and selectivity of MNA assay, various concentrations of human CRP, mouse CRP, IGFBP-2, TNFRII, VCAM-1, or bovine serum albumin (BSA) solutions were prepared in PBS solution containing 2% BSA, and then added onto MNs of MNA as described above, followed by 2-hour incubation at room temperature (RT). Mice CRP and BSA were used as negative controls to examine the selectivity of assay. Next, the MNA was rinsed with washing buffer every 5 minutes for a total of three times to remove the non-specific binding. The biotinylated detection antibodies for CRP, IGFBP-2, TNFRII, or VCAM-1, which were added to the MNA sequentially and incubated for I hour, followed by washing with washing buffer every 5 minutes for a total of three times and incubation with streptavidin-HRP for 20 minutes. The MNA was then washed every 5 minutes for a total of three times. Gel was tailored to desired size, and air dried. The dried gel was then immersed in colorimetric substrate solution until it was saturated. The MNA was inserted into the gel and pressed firmly for 10 minutes, after which the gel was removed and washed using distilled water and carefully transferred to a plastic dish for imaging. The images of stained gel were captured by a microscope and analyzed by ImageJ. For single biomarker measurement, the serum samples were diluted 1000 times for measurement of IGFBP-2, TNFRII, and VCAM-1 levels, while serum samples were diluted 20000 times and 40000 times were employed for CRP detection. For multi-biomarkers measurement in a single MNA, serum samples at a dilution of 1:1000 in reagent diluent, were added into the MNs with 2-hour incubation at RT.

### ELISA Assay Validation

Serum CRP, IGFBP-2, TNF-RII, VCAM-1 levels of the patients with lupus and healthy controls were measured using commercial ELISA kits. The ELISA assays were conducted according to the manufacturer's instructions. For dsDNA ELISA assay, each well of a 96 well plate is pre-coated with 0.1 mg/mL of mBSA and incubated for 30 minutes at 37° C. After the plate was washed with PBS for two times, 200 ug/mL of dsDNA was added and incubated for 30 minutes at 37° C. The washing step was repeated to remove unbinding reagents. The serum samples were diluted 100 times and incubated for 2 hours. After incubation with biotinylated human IgG antibody (2 hours) and streptavidin-IRP (20 minutes) at RT, TMB substrate was added for 20 minutes. The reaction was stopped by addition of stop solution. The results were read on a UV spectrophotometer (Epoch, Biotek, Winooski, VT, USA). The data were analyzed by Graphpad Prism 9. The sample samples at a dilution of 1:100 in reagent diluent were used for IGFBP2, TNFRII, ds DNA, while for VCAM-1 detection, the samples were diluted 1600 times. The samples at different dilutions of 1:500, 1:10000, and 1:50000 in reagent diluent were utilized to measure the CRP level.

### Statistical Analysis

We used mean signal intensity of blank plus 3 times the standard deviation of the blank (36) to calculate corresponding biomarker concentration as the LoD. The statistical difference between two groups was analyzed by a two-tailed unpaired t-test. All graphs were plotted by GraphPad Prism version 9.0. All statistical tests were conducted using GraphPad Prism version 9.0. a P value<0.05 was considered statistically significant for all statistical tests.

To assess the diagnostic ability of selected biomarkers and their combinations, a logistic regression model was employed to classify SLE patients from healthy controls using single or combinations of up to five biomarkers. To prevent overfitting and ensure generalizability, a five-fold cross-validation method was implemented using the scikit-learn package in Python 3.9. Biomarker values were normalized based on their mean and standard deviation. 5-fold cross validation was used by randomly dividing the samples into five groups, selecting four groups of the samples as a training set to establish a model, while utilizing the remaining samples as a training set. This process was repeated one time, and classification performance metrics, including area under the curve (AUC), accuracy, sensitivity, and specificity, were reported as the average of all five folds during cross-validation for both training and testing sets (Table 4). Subsequently, a final logistic regression model was built using all the data to generate receiver operating characteristic (ROC) curves, as well as to calculate the AUC and optimal cutoff values which was defined as maximization of the sum of sensitivity and specificity.

### Results and Discussion

### MNA Fabrication and Surface Modification

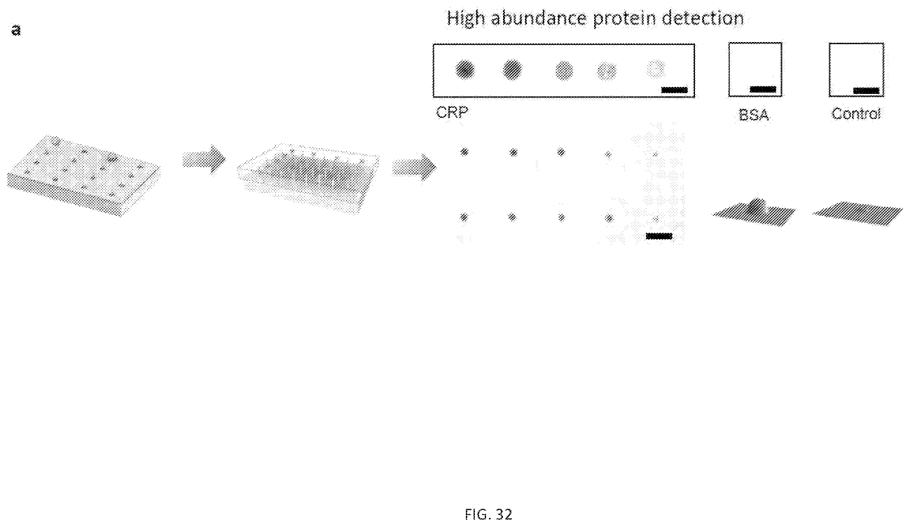

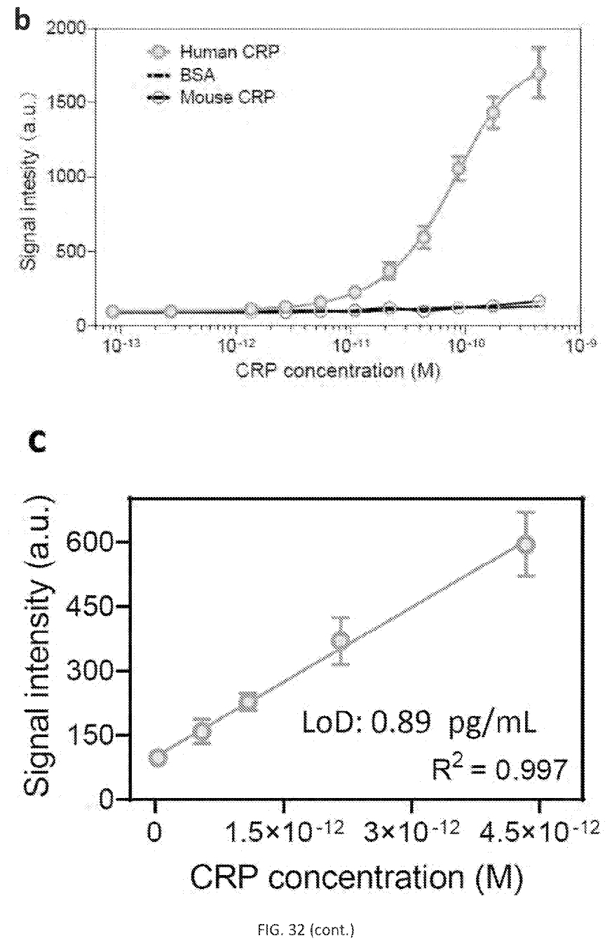

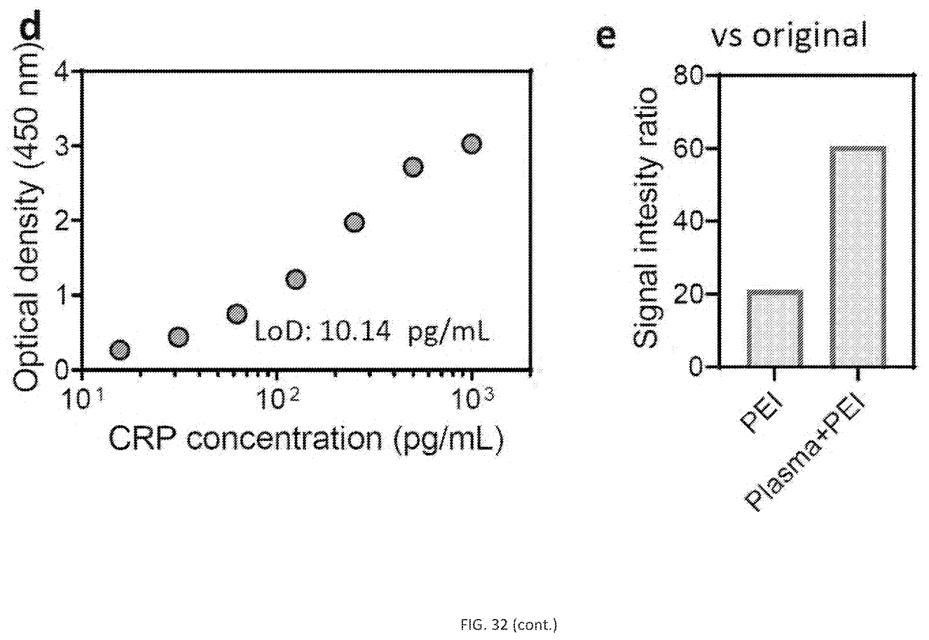

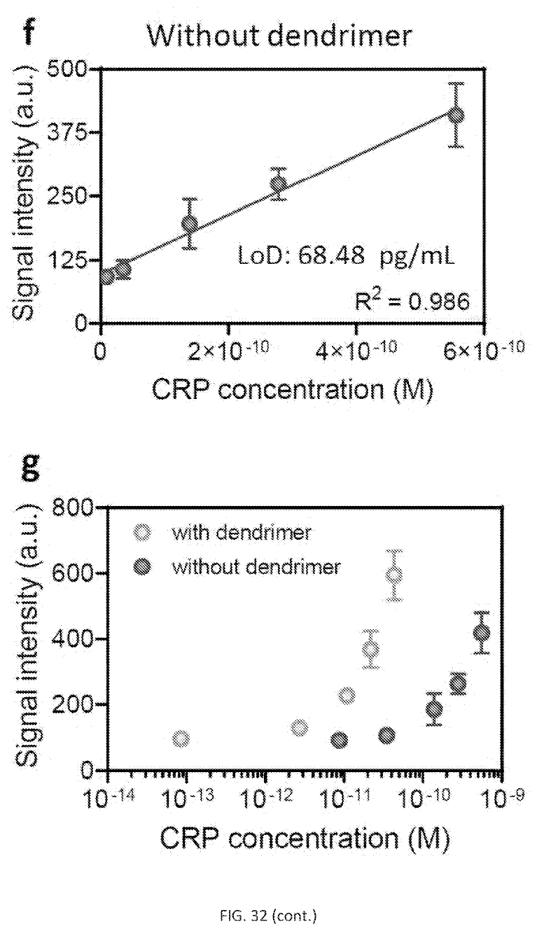

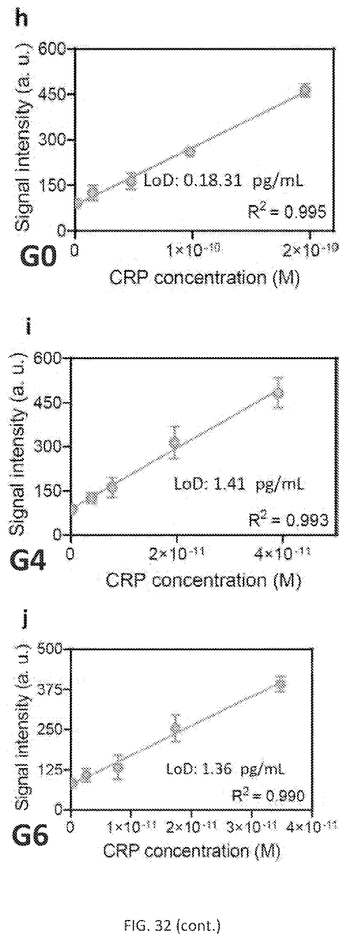

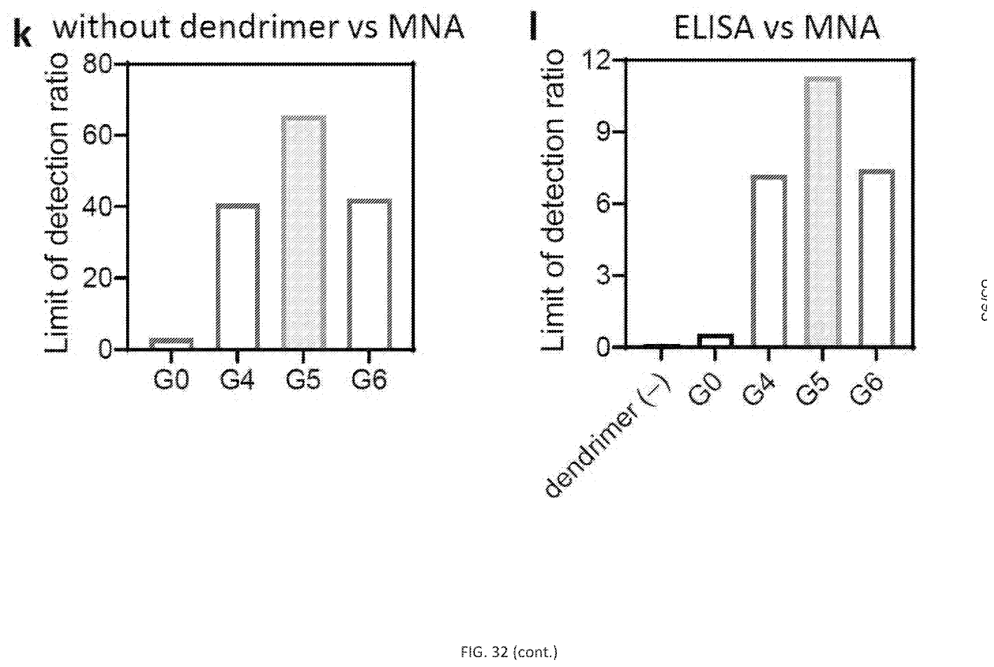

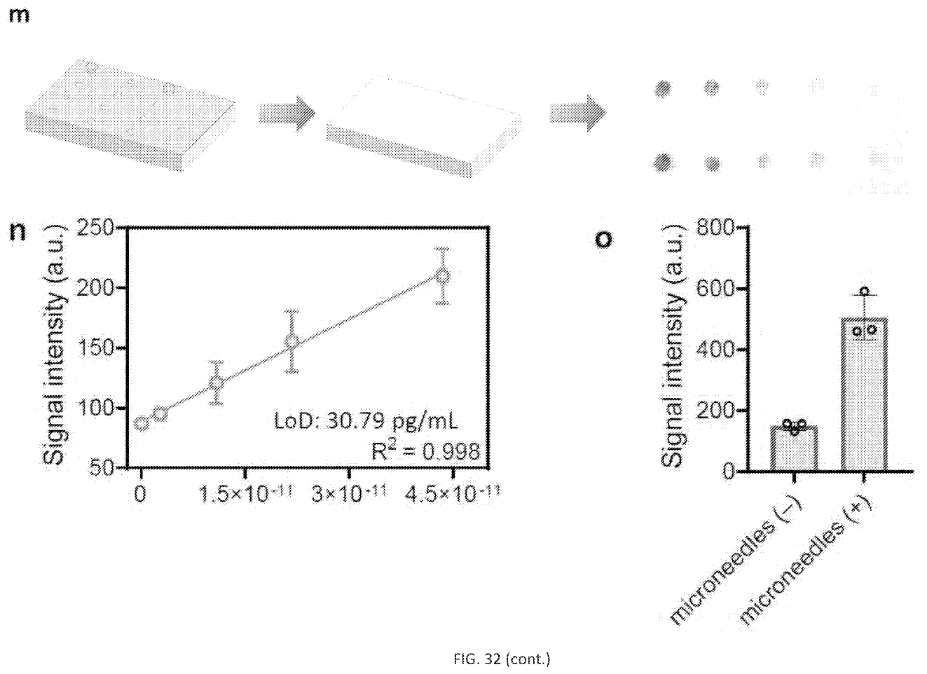

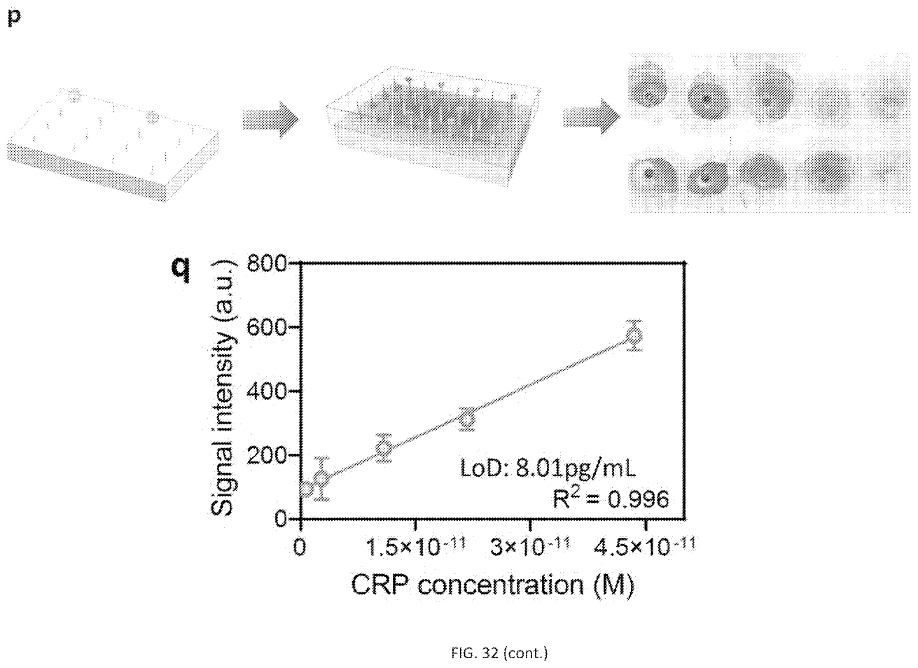

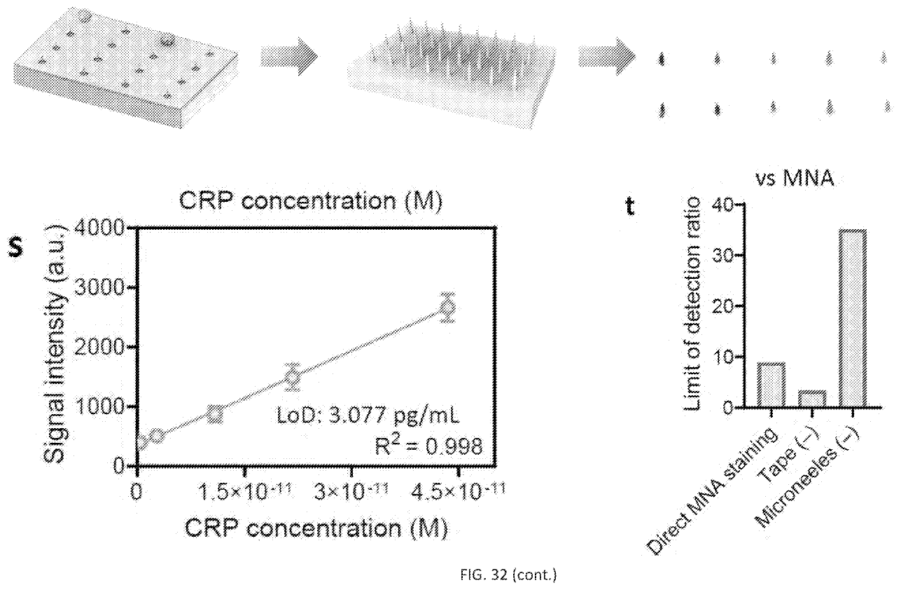

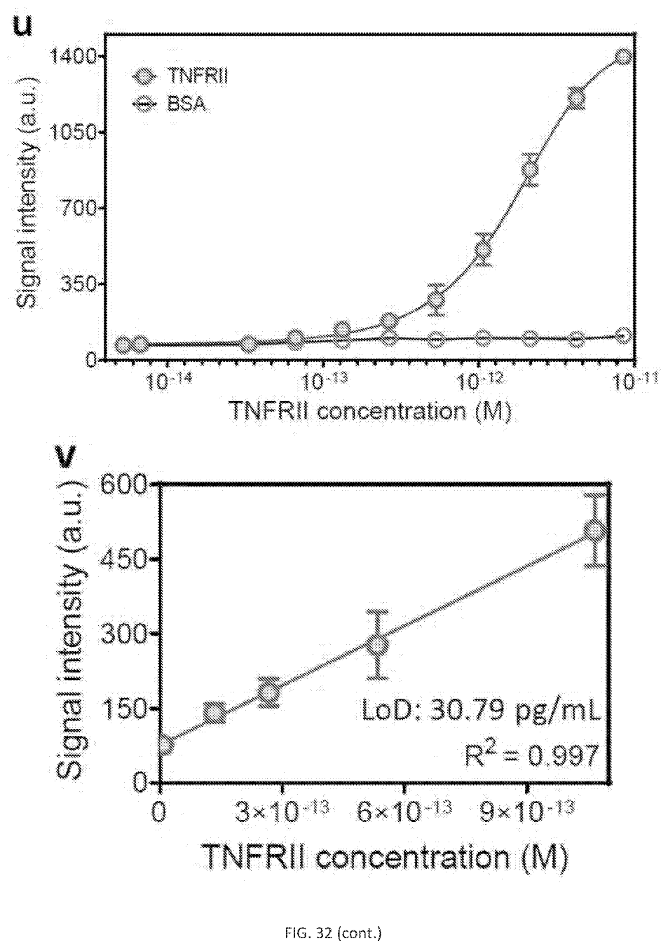

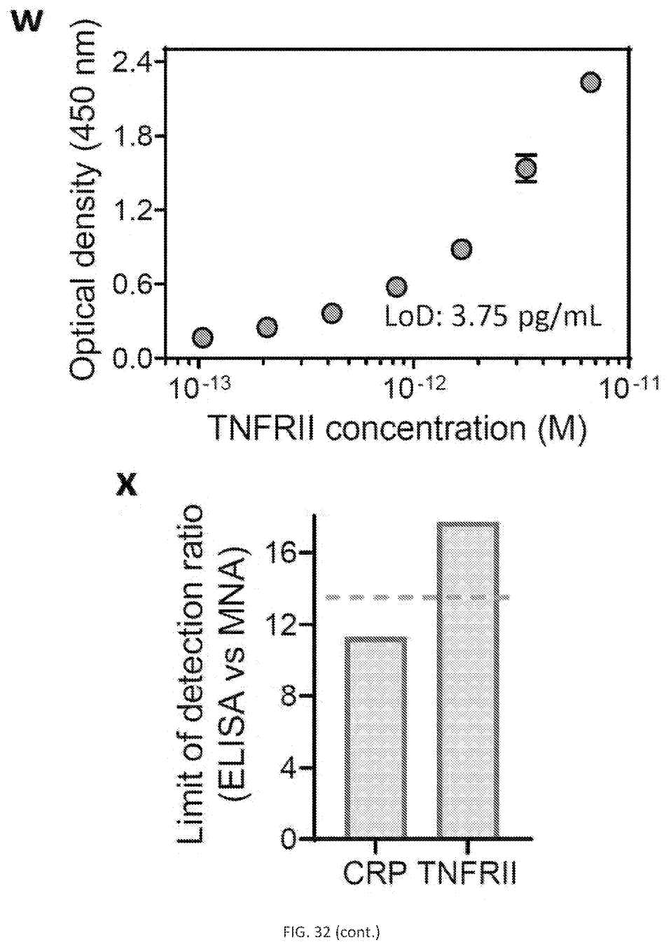

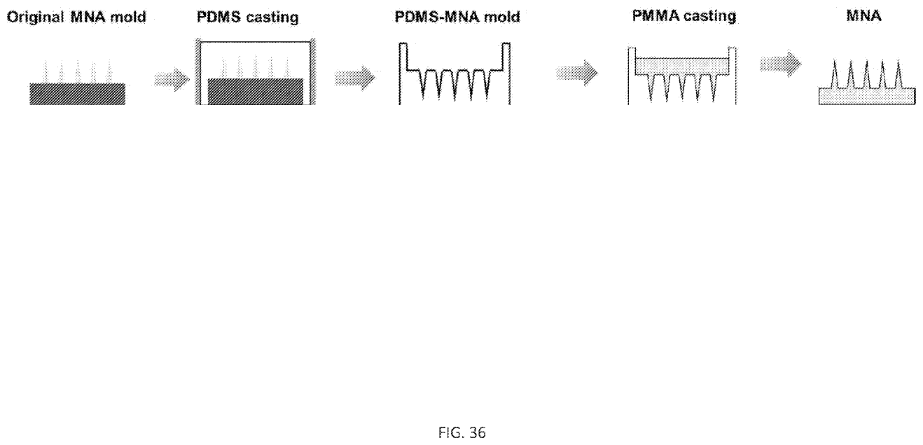

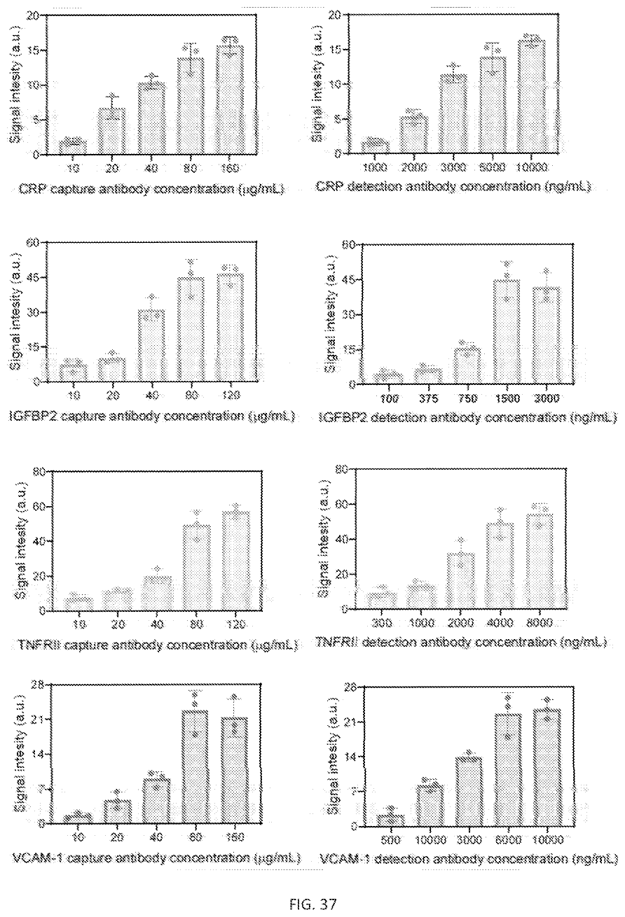

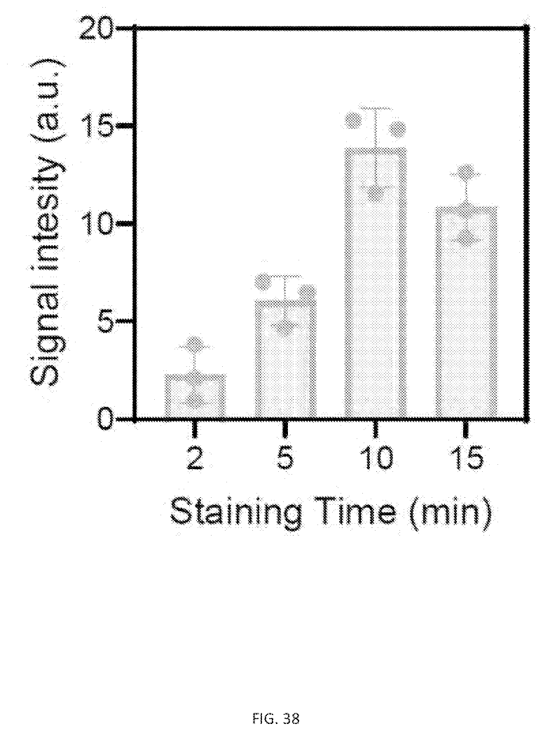

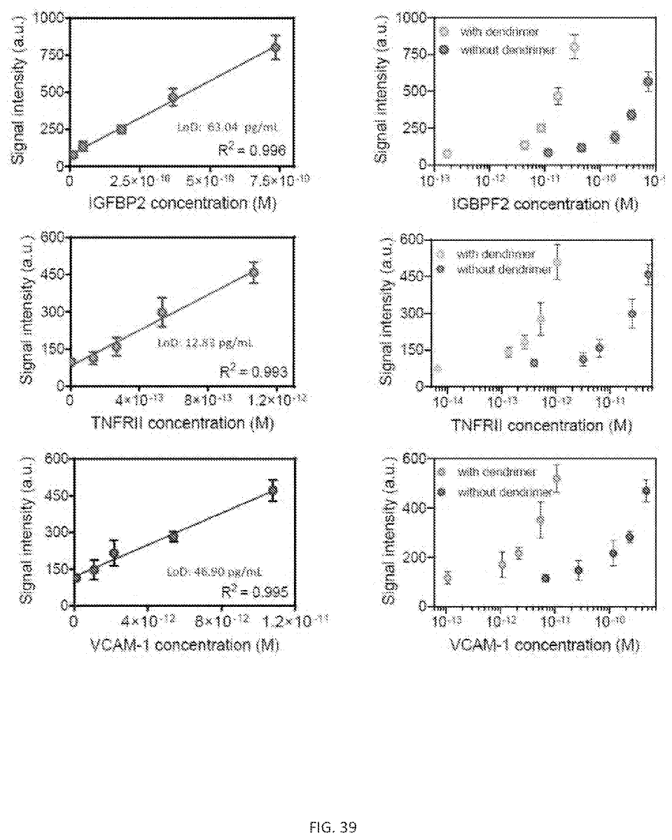

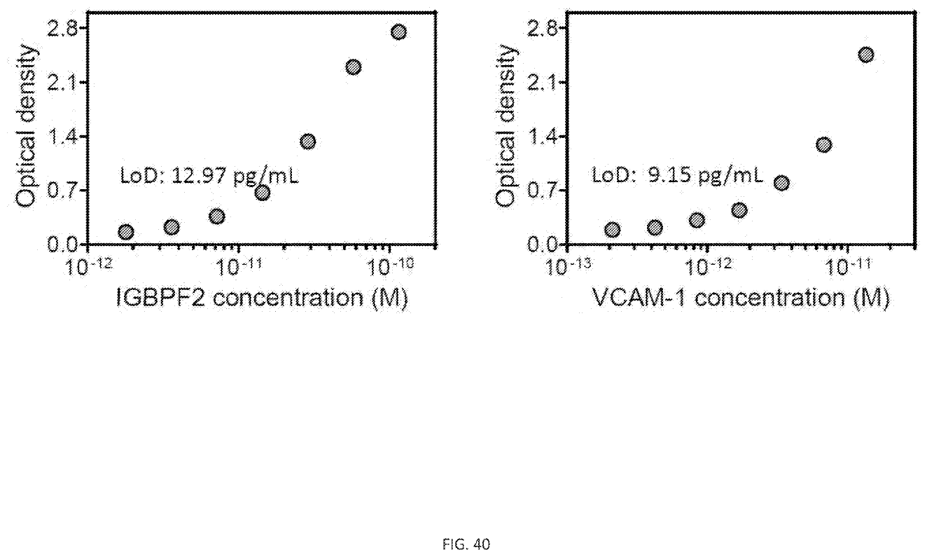

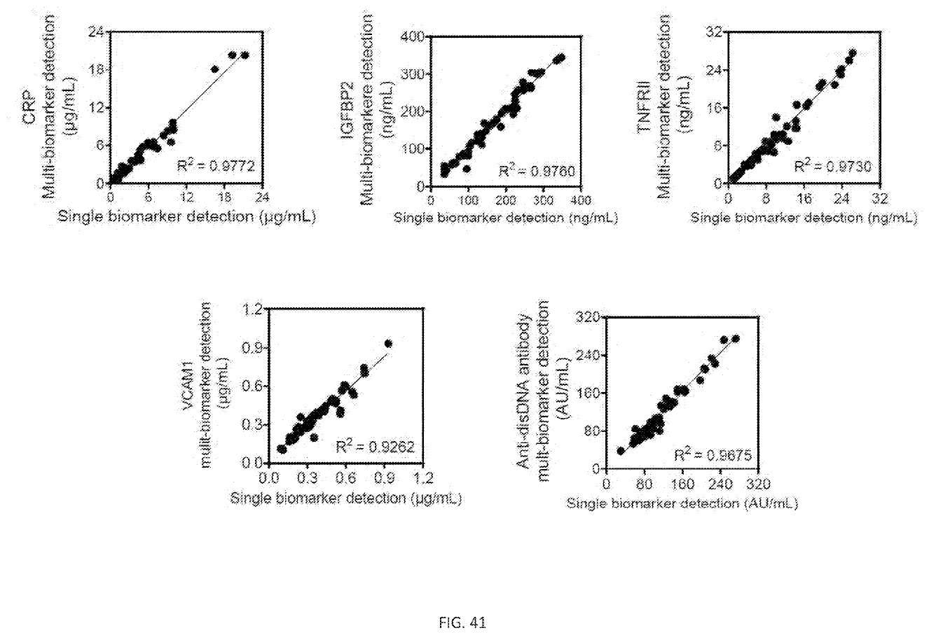

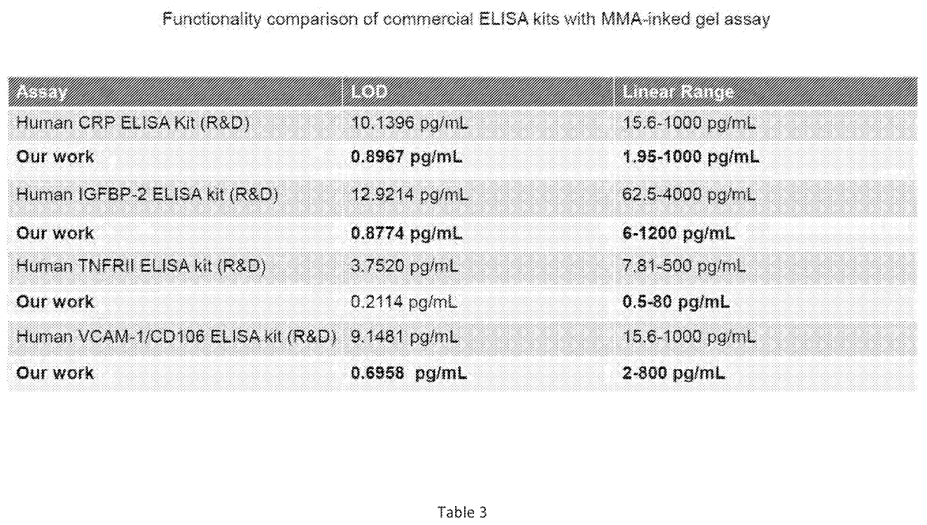

The design, fabrication, and surface modification strategy of MNAs is shown in FIG. 36 and FIG. 31a. Considering that MNA is needed to be inserted into a gel in MNA gel assay and future application at PoC, poly(methyl methacrylate) (PMMA) which is a synthetic polymer with sufficient mechanical strength and related low cost, was used in fabrication of MNA. MNA was constructed via casting PMMA solution in a female PDMS-MNA mold that was made by a male MNA mold FIG. 31. We can find that the MNA formed by microneedles with cone-shape was successfully fabricated as shown in scanning electron microscope (SEM) image (FIG. 31b), and the average length of microneedle was 500.0±16.27 μm, while the diameter of microneedle top and base were 10.2±0.69 μm and 181.0±6.78 μm, respectively. Since there are no functional groups for covalent conjugation with biomolecules, we employed branched polyethyleneimine (PEI), a cationic polymer containing numerous amine functional groups, to modify the surface of MNA, providing amine-reactive chemical groups for conjugation with amine groups or carboxyl groups of targeted molecules via different coupling reactions. However, the amount of PEI that is physically adsorbed onto PMMA surface via electrostatic force is limited. Due to the difficulty of PMMA surface modification resulted by low surface energy and hydrophobicity, oxygen plasma treatment, a well-established approach for increase in surface hydrophilicity and energy, was conducted to produce more active functional groups on PMMA surface for increase of PEI modification efficiency. To examine the effect of plasma treatment, we selected a extensively investigated inflammatory biomarker C-reactive protein (CRP) that is produced by hepatocytes as a template, and carried out CRP detection on three MNAs with difficult surface medications through immobilization of the same amount of CRP protein followed by addition of detection antibody. As shown in FIG. 32e, compared to the original MNA which CRP was physically absorbed on surface, it is notable that the MNA with plasma treatment and PEI modification exhibits 60-fold higher in signal intensity. While the signal intensity of MNA with plasma treatment and PEI modification was also observed 3-fold higher than that of MNA with only PEI modification. However, some research groups have explored CRP detection using microfluidic chip made by PEI-modified PMMA, yet its performance was not competitive to ELISA, which might be contributed to the insufficient reaction activated groups for capture antibody conjugation. Therefore, the available amount of reaction activated groups on surface is of importance, requiring further modify the PEI coated PMMA.

To maximize reactive sites for immobilization of capture elements, we further decorated the surface of PEI-coated MMA with dendrimers (FIG. 31a). Dendrimer is a highly symmetric and stable polymer with a spherical shape surrounded by a high density of functional groups at multiple branch ends, providing a large area for immobilization of biomolecules, thereby it has been explored in the fabrication of various biosensor including microarray, electrochemical sensors, and surface Plasmon resonance sensor. There are different types of generation of dendrimer, according to generations of polymer branching structure by the successive reaction steps. Dendrimer generation 4 has been exploited to modify ELISA plate coating by polyethylene glycol (PEG) for measurement of tumor necrosis factor-alpha (TNF-α) and IL-6, and they were reported to have LoD superior to commercial ELISA kits. In this study, we performed the CRP MNA gel assay on MNAs which were modified with different generations of dendrimers (G0, G4, G5, and G6) and immobilized CRP capture antibody to determine the best candidate. The result can be seen in FIGS. 32k and 32l, which displays that the MNA decorated with dendrimer G5 had the best performance. Notably, the LoD of MNAs with dendrimer G5 and without dendrimers are 0.89 pg/mL and 68.48 pg/mL, respectively, indicating MNA with dendrimer G5 showed more than 75-fold lower LoD compared to MNA without dendrimers (FIG. 32k). Both showed good linear of 1.95 to 1000 pg/mL and 200 to 12800 pg/ML for MNA with and without dendrimer G5, respectively. Of note, the clinical cut-off level for is 0.25 ng/mL, indicating that the sensitivity of MNA assay meets the clinical standard. As expected, all the MNA shows an average 67-fold improvement by in LoD in comparison to that of MNA without decoration of dendrimer, especially for dendrimer G4, G5 and G6 (FIG. 32k). Interestingly, the improvement of LoD of MNA increased with the increased dendrimer generations, while it decreased for MNA decorated with dendrimer G6, which might because the large size of dendrimer results in the limited number of decorated dendrimers on the surface of microneedles and the steric hindrance for subsequent molecular binding.

One Microneedle Immobilized with One Capture Element

Taking advantage of the large surface area of microneedle, on-needle detection of biomarker via individual immobilization of capture element is expected to have high sensitivity. However, most reported MNA-based biosensors typically can only detect one biomarker. For instance, some have reported an MNA assay by using a gold nanorod enhanced fluorescent label for on-needle detection of one specific biomarker. Although it showed the ability to detect a low level of biomarker, the signal capture and analysis relied on confocal microscope. To achieve on-needle detection for multiplex biomarkers in one MNA, each microneedle should be immobilized with one specific capture antibody, which is extremely difficult due to its tiny-size. Despite a few studies recent reported MNAs for multiplex biomarker detection by using MNA, to date, none of these studies reported one specific biomarker capture element modified one microneedle. For example, in an MNA for multiple biomarker detection, microneedle-shaped polymeric “shell” might act as a barrier for biomarker capture and recognition by detection antibody because the capture antibodies were immobilized photonic crystals (PhCs) which were used as barcodes and loading inside the microneedles. Besides, the construction process of microneedle array might also affect the capture antibody. A more recent study reported fabrication of a wearable microneedle array for real-time monitoring of multiple metabolites, but the multi-analytes detection was achieved by building microneedles in different modules of 3-electrode electrochemical system for detection of different kind of biomarkers instead of a single needle modification. The high cost of complicated and construction process limits its application at PoC. In this study, immobilization of multiplex capture elements on a single MNA in one capture element on one MN fashion represents the first instance of surface modification of MNA for multiplex biomarkers detection. A double-side tape with a hydrophobic surface was treated with laser to punch an array of tiny holes precisely aligning with the microneedles in the MNA. MNA base was covered by the tape, and each hole serves as microcontainer for the corresponding microneedle on MNA, filling by a coupling reaction mixture of EDC/NHS and capture antibody for specific capture antibody immobilization on the corresponding microneedles (FIG. 31c). This novel approach might open a new avenue for on-needle detection of multiple biomarkers.

Compared to the microarray that capture elements are immobilized on a glass slide by injecting a drop of capture element solution on a flat spot, a larger surface area of microneedles allows for the immobilization of more capture element molecules, resulting in a significant improvement of sensitivity. The use of a tape in the method of one microneedle immobilized with one capture element, allows us to compare the MNA assay with and without microneedles on a PMMA base surface. As shown in FIG. 32m, we prepared a PMMA base without microneedles with surface modification by PEI and dendrimer decoration, and punched an array of tiny holes with the similar diameter of base of microneedles on a double-side tape with a hydrophobic surface. The PMMA base without microneedles was covered with prepared a double-side tape and immobilized CRP capture antibody, followed by addition of CRP solution with various concentration, biotin-detection antibody, Streptavidin-HRP, and the colorimetric substrate. The kinetics and standard curve were plotted (FIG. 32m, right), and LoD was determined as 30.79 pg/mL. Although the surface of individual MNs is only 5.6-fold larger than that of a microneedle base, MNA gel assay detecting 0.89 pg/mL has a 35-fold higher sensitivity than that of MNA gel assay without microneedles (a flat base vs. a 3D MN).

Moreover, a double-sided tape was used to cover the MNA base surface during the process of microneedles coated with a capture element, preventing the background signal, and increasing the sensitivity of MNA gel assay. As shown in FIG. 32p, we observed that the background signal in the gel was obvious if the MNA gel assay was conducted by using an MNA without double-side tape protection, resulting in a 9-fold lower sensitivity, in comparison with the MNA gel assay. Besides, direct imaging of microneedles for quantification of biomarkers is the predominate method in previous studies based on MNA. We also performed direct imaging of microneedles assay on the MNA to detect CRP. As expected, direct imaging of microneedles assay had a higher LoD level (3.08 pg/mL) (FIG. 32r).

MNA gel assay for low abundance biomarker detections.

To demonstrate the ability of MNA gel assay for the detection of molecules with a broad range from high abundance biomarkers to low abundance biomarkers, tumor necrosis factor receptor type II (TNFRII) was selected as a representative of low abundance biomarkers for detection. The biomarker is expressed by T lymphocytes with a serum level lower than 100 ng/mL in healthy controls and can inhibit activations of tumor necrosis factor α (TNFα). FIG. 32 u,v displays the kinetic of MNA gel assay in the presence of various concentrations of human TNFRII or BSA in PBS, and its standard curve was highly linear. An LoD was found to be 18-fold lower than that of ELISA, suggesting the high sensitivity of MNA gel assay for low abundance biomarker detection.

### MNA Gel Assay for Multi-Biomarker Detection

Antinuclear antibodies (ANA) test, the current European League Against Rheumatism/American College of Rheumatology classification criteria for systemic lupus erythematosus (SLE), has a limited specificity (57%), representing an example that the use of a single biomarker is insufficient for disease diagnostics. SLE, a chronic and inflammatory autoimmune disease presenting various abnormalities in a wide range of organs for different patients, might eventually lead to tissue and organ damage caused by the long-term attack of the patient's own immune system if the disease activities are not well-controlled. A strong association is found between organ damage and elevated risk of SLE mortality, especially cardiovascular and renal damage. For instance, lupus nephritis is the leading cause of death of SLE. SLE predominantly affects women and has been recognized as the 6th leading cause of death in young women aged between 25 to 34 years in the United States. Therefore, continuous disease activity monitoring during the treatment, which can guide further effective therapy and disease management, is critical in minimizing the risk of organ damage and reducing mortality. To demonstrate the proof of concept of MNA gel assay in multi-biomarkers quantification to improve disease diagnosis accuracy and potential clinical application, we rationally selected five biomarkers, and investigated single and combinations of the five biomarkers for SLE diagnosis.

Immune disorders cause a large number of varying types of autoantibodies capable of attacking body's normal tissues or organs, such as anti-double-stranded DNA antibody (anti-dsDNA), in patients with active lupus, leading to the formation of immune deposits and vascular inflammation in many organs. During this process, inflammatory molecules stimulate endothelial cells to express E-selectin that binds to carbohydrate groups of leucocytes. As a result, leucocytes adhere to endothelium and up-regulate adhesion molecules such as vascular cell adhesion molecule-1 (VCAM-1) for facilitating transmigration of inflammatory cells. Certain researchers have reported that the VCAM-1 levels in patients with active lupus were elevated. Although the exact mechanisms underlying the increased human insulin-like growth factor binding protein-2 (IGFBP-2) in lupus are unknown. Some groups have found significantly higher levels of IGFBP-2 and tumor necrosis factor receptor type II (TNF-RII) in patients with active lupus over the control groups, suggesting that they could be the biomarkers of active lupus. IGFBPs might be also involved in IGF-1-dependent or IGF-1-independent signaling pathways to regulate immune cell proliferation. TNF-RII, which is expressed by T lymphocytes, can inhibit activations of tumor necrosis factor α (TNFα) that is regarded as a protective role in lupus. It participates in regulation of B-cell activation and related production of autoantibodies, a hallmark for the development of lupus. Therefore, these five biomarkers (human CRP, IGFBP-2, TNF-RII, VCAM-1, and anti-dsDNA antibody) were selected for the investigation to demonstrate the proof of concept of MNA-inked gel assay in multi-biomarkers quantification.

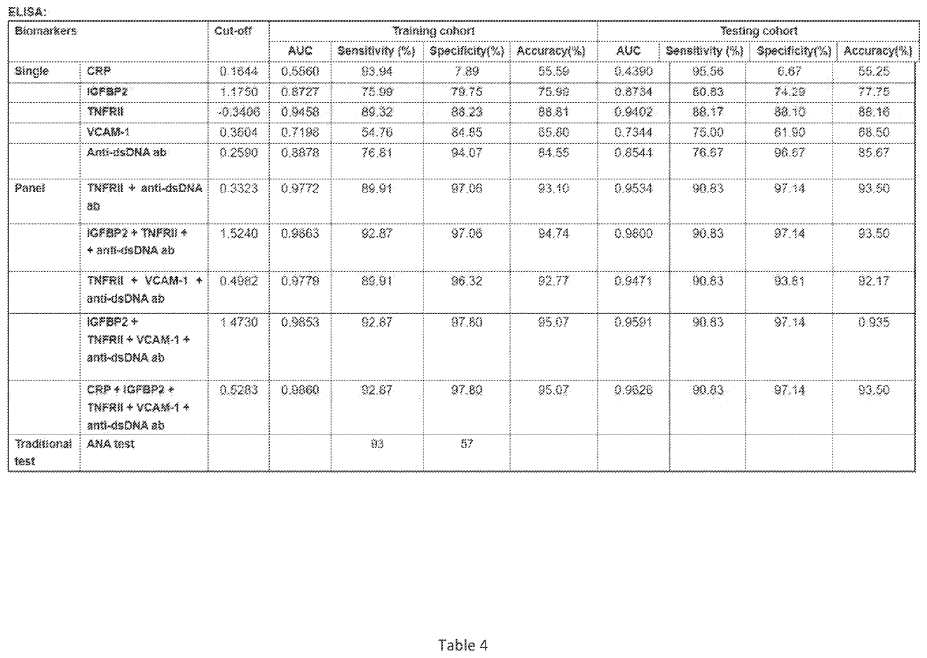

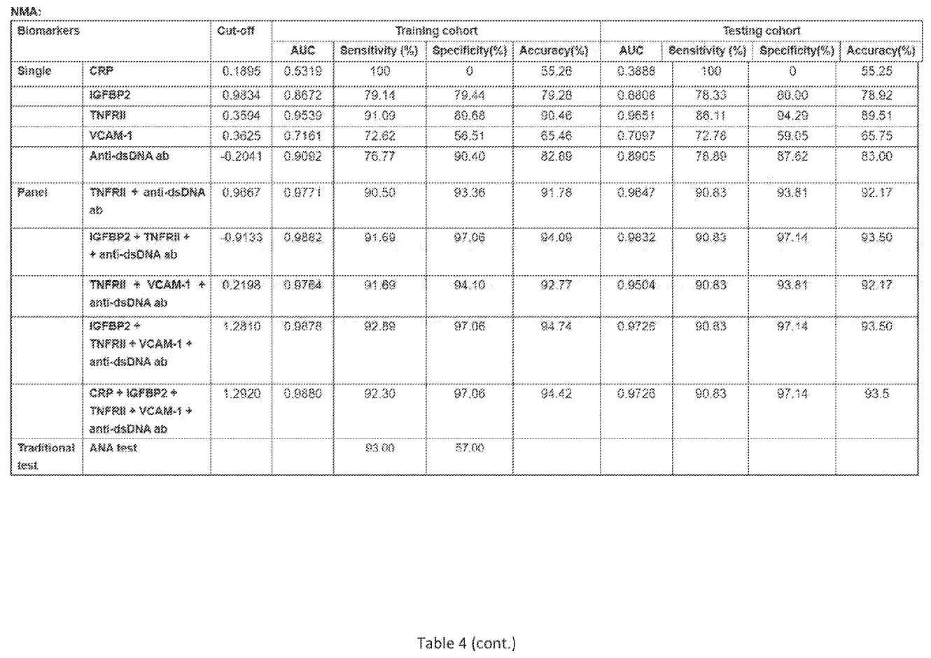

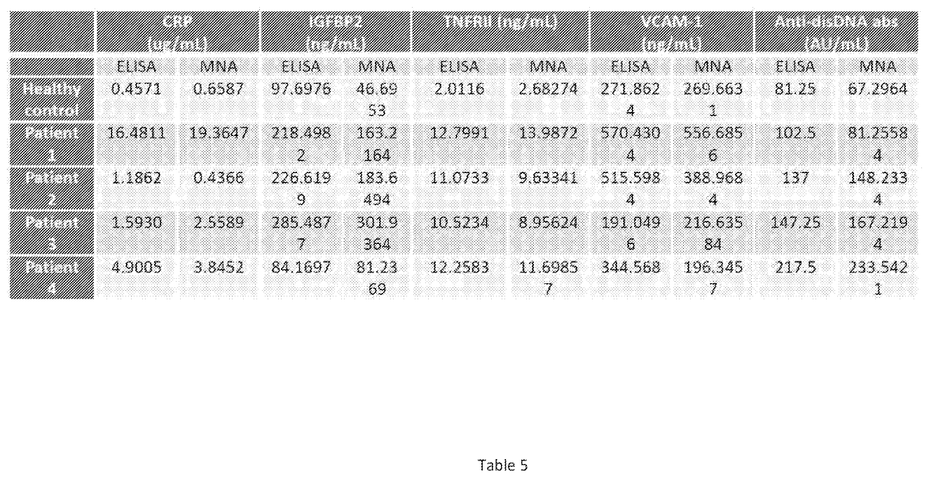

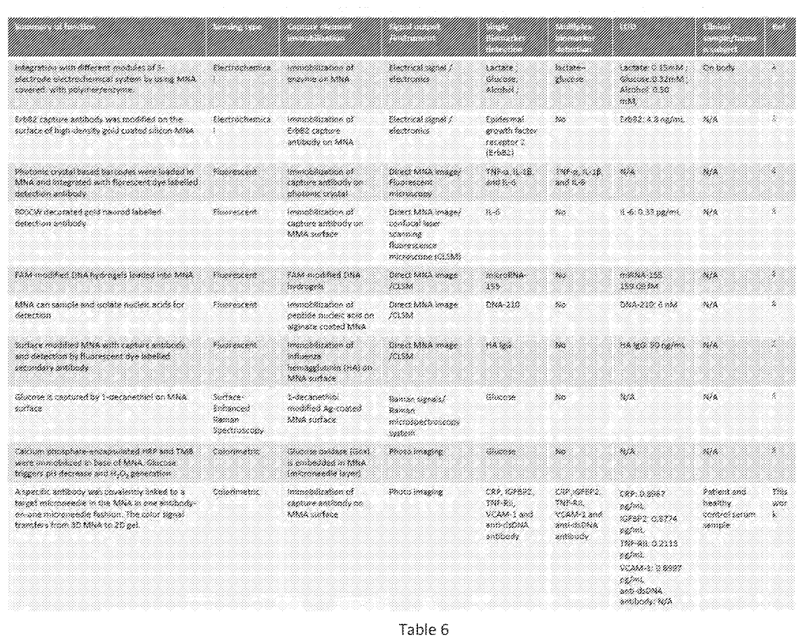

We first evaluated a pair of capture and detection antibodies for each of these biomarkers and compared the results between the MNA-inked gel assay and commercially available ELISA kit. To this end, we constructed MNAs for single biomarker detection. To achieve the best performance, we determined optimal conditions after carefully examining how various concentration of capture antibodies, detection antibodies, the color substrate incubation time impact the functionality of the of MNA-inked assay, and the results are shown in FIGS. 40-41.

In FIG. 32a, a representative gel image of one MN row was given in a one microneedle row, and the spots on the gel corresponded to human CRP in a range from 80 to 1,000 pg/mL in PBS compared to negative controls 500 pg/mL of BSA and PBS. FIG. 32a, which is the image is enlarged from FIG. 32a, displays duplicate samples of each concentration of CRP in two rows of the MNA, indicating its reproducibility and specificity. It is notable that the background is clear without unspecific binding, which is largely attributed to our unique immobilization of multiplex capture elements on a single MNA in one capture element on one microneedle fashion, thus the capture elements were only in presence on surface of microneedles. The cone shape of the microneedles results in round shape spots with an approximate diameter of 207 μm, and their color intensities and sizes are proportional to the human CRP concentrations in the test region, in good agreement with the results of 3D images of stained spots on gel in FIG. 32c. The size of spots is closely similar to the base of microneedle, revealing that the blue color products resulting from catalyzed color substrate by HRP did not diffuse in gel, and well surrounding the sites where microneedles were inserted.

To evaluate the sensitivity and specificity of MNAs for single detection of these five biomarkers, various concentrations of target biomarker, and control solutions in PBS were employed to establish the binding kinetics. FIGS. 32a-32d (left) shows the dose-dependent responses of MNA-inked gel assays to indicate concentrations of human CRP, IGFBP-2, TNF-RII, and VCAM-1 in PBS. FIGS. 32a-32d (right) exhibits the calibration curves of IGFBP-2, TNF-RII, and VCAM-1 in PBS of assay, which are used to identify the LoD. The calibration curves of human CRP, IGFBP-2, TNF-RII, and VCAM-1 offer the LOD of the corresponding biomarkers 0.8967 pg/mL (3.887×10⁻¹⁴ M), 0.8774 pg/mL (2.5204×10⁻¹⁴ M), 0.2118 pg/mL (2.8246×10⁻¹⁵ M), and 0.6997 pg/mL (9.4438×10⁻¹⁵ M), respectively, and the linear ranges from 1.95 to 1000 gp/mL, 6 to 1200 pg/mL, 0.5 to 80 pg/mL, or 2 to 800 pg/mL, respectively, (FIG. 32e). Table 3 summarizes the comparison of commercial ELISA assays of human CRP, IGFBP-2, TNF-RII, and VCAM-1 with the results of our MNA-inked gel assay. Clearly, in comparison with the commercial ELISA kits for human CRP, IGFBP-2, TNF-RII, and VCAM-1, MNA-inked gel assay increased the detection sensitivity by 11.30-fold, 14.78-fold, 17.74-fold, and 13.07-fold, respectively (FIG. 32f). The LoD values of our colorimetric assays are an order of magnitude lower than those of ELISA kits, suggesting an average of 14.22× higher sensitivity than ELISA in addition to a 50× less amount of reagents used for cost-effective. MNAs with decoration of dendrimers were found to increase the sensitivity of the assay by about 76.36-fold, 71.85-fold, 60.70-fold, and 67.025 fold for human CRP, IGFBP-2, TNF-RII, and VCAM-1, respectively, compared to MNAs without decoration of dendrimers (FIG. 32g). Furthermore, the high specificity of the assay was demonstrated by negligible signal on the gel upon the addition of negative controls buffer solution containing mouse CRP or BSA.

Serum samples collected from patients with lupus (N=42) and healthy controls (N=34) were next evaluated by the MNAs for single biomarker detection to validate the clinical potential of MNA-inked gel assays. ELISA was run in parallel for comparisons. The data were shown in FIG. 32h, highly correlation of the two assays and similarity of the two results. Thus, the same conclusion can be drawn from the two assays: i.e. in comparison with healthy controls, levels of IGFBP2, TNF-RII, VCAM-1 and anti-dsDNA antibody rose significantly in the serum of patients with lupus and especially, the level of TNF-RII and anti-dsDNA antibody were significantly higher in patients with lupus than that in healthy controls. However, there was no significant difference in the level of CRP between SLE patients and healthy controls.

We performed a paired correlation analysis of the results between ELISA and MNA-inked gel assays, the data obtained from the same patient using MNA assay or ELISA were used for pair test of correlation. As shown in FIG. 32h, the high coefficient of determination R² values of 0.9503, 0.9301, 0.9548, 0.9119, and 0.9212, for CRP, IGFBP2, TNF-RII, VCAM-1, and anti-dsDNA antibody, respectively, was found between ELISA and the MNAs, suggesting that the reliability, sensitivity, and accuracy of the MNAs for single biomarker detection are comparable to clinically used ELISA kits. It is noteworthy that the levels of these five biomarkers between the patient group and healthy controls overlapped, leading to the inaccuracy in discriminating patients from healthy controls. Apparently, multi-biomarker detection is required for more accurate diagnosis.

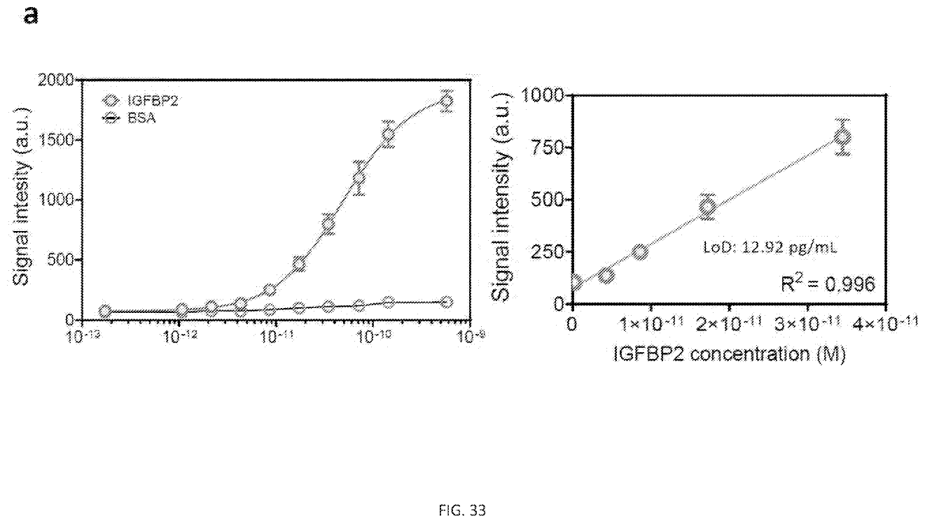

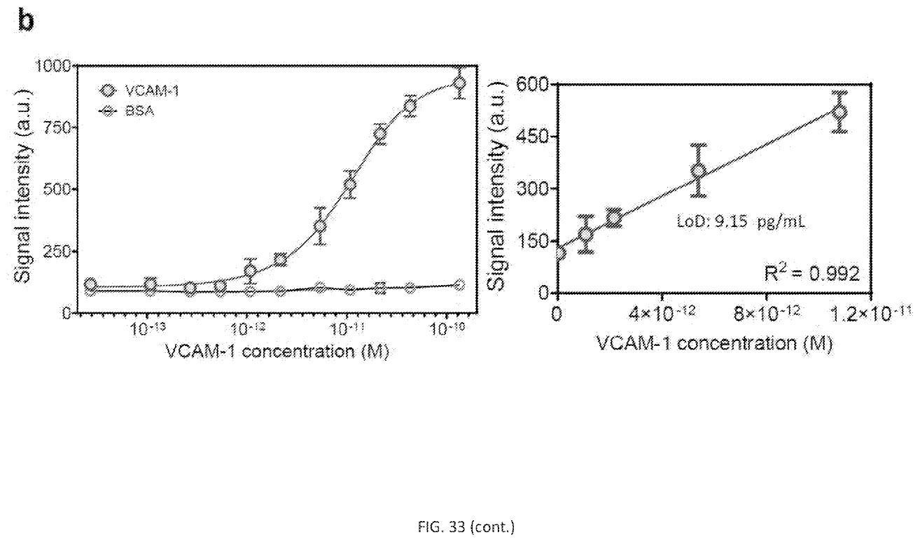

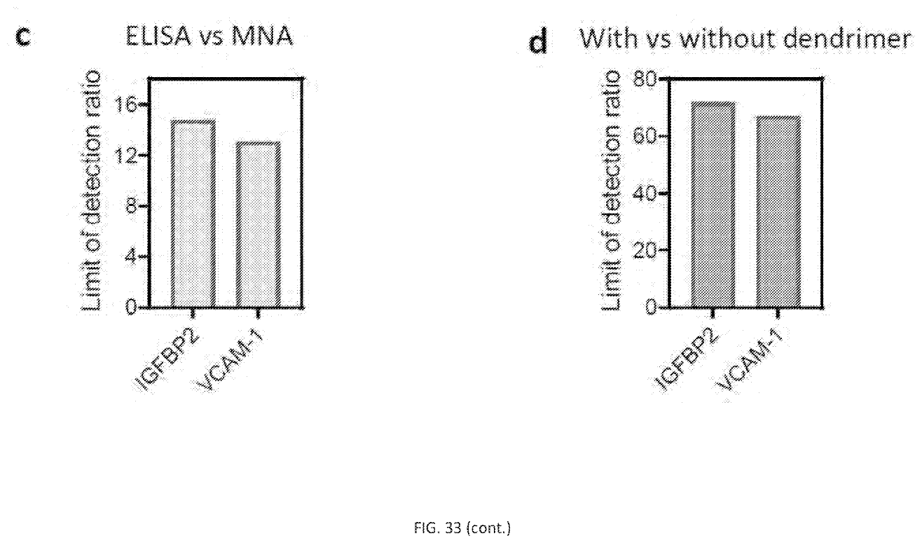

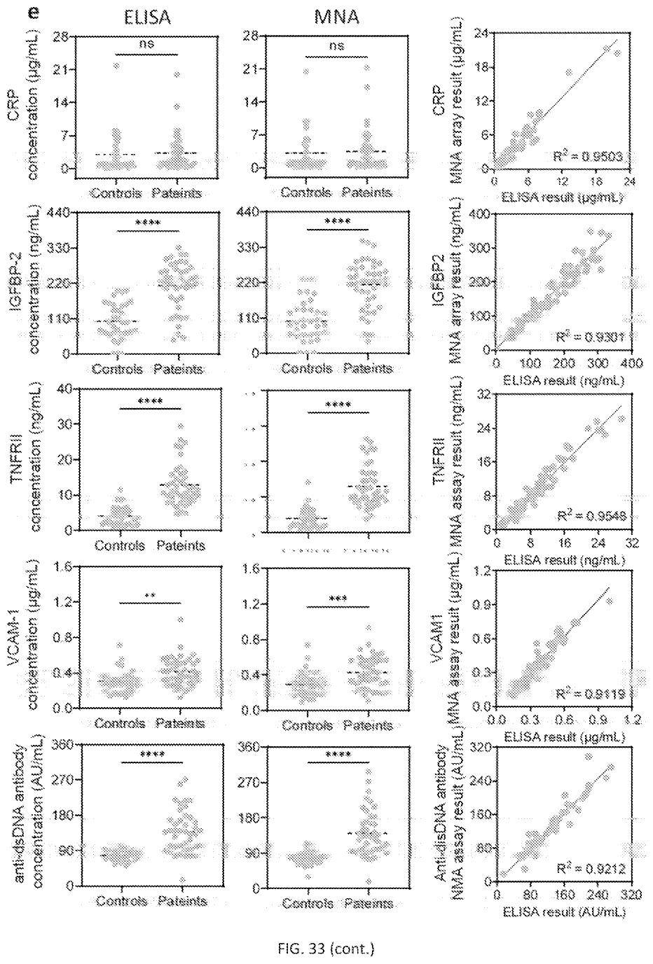

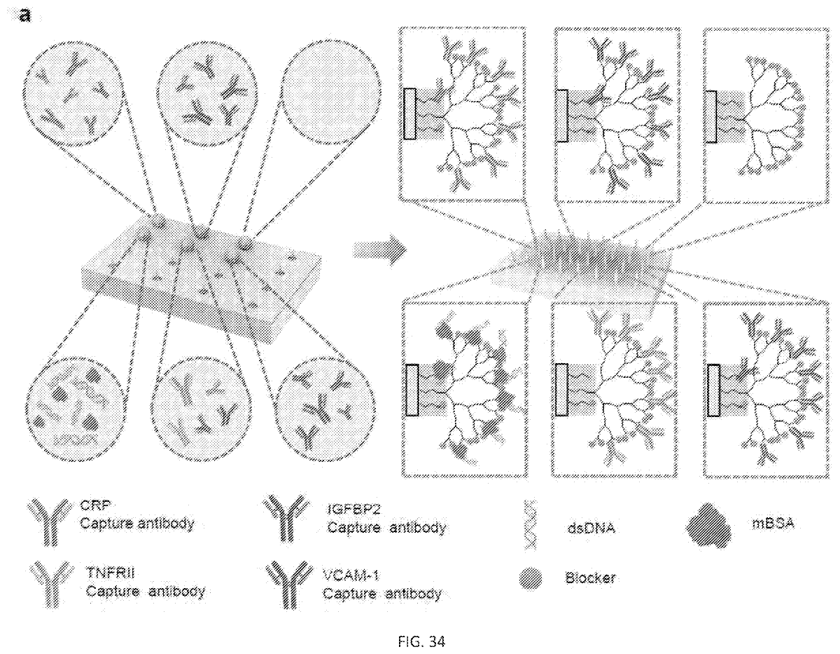

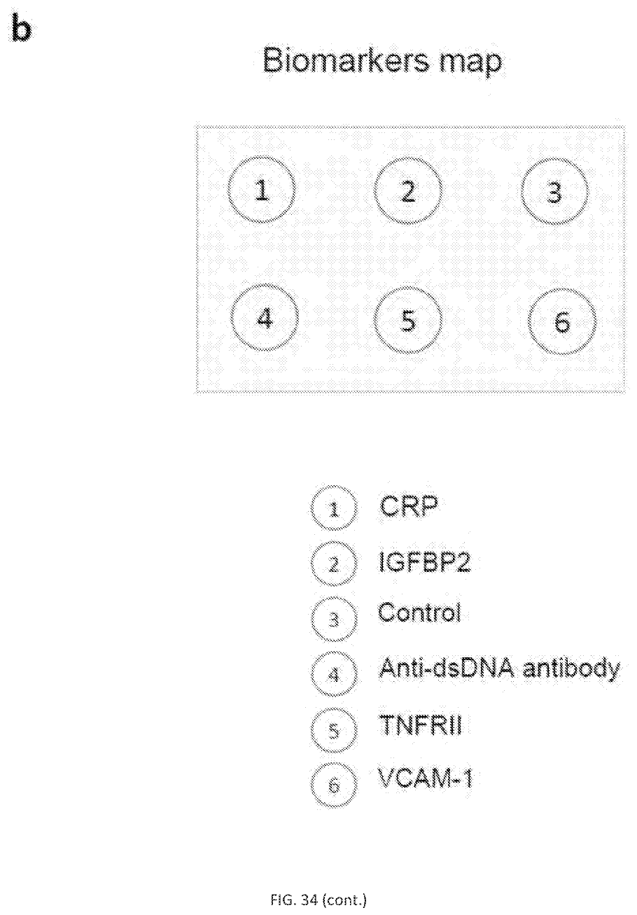

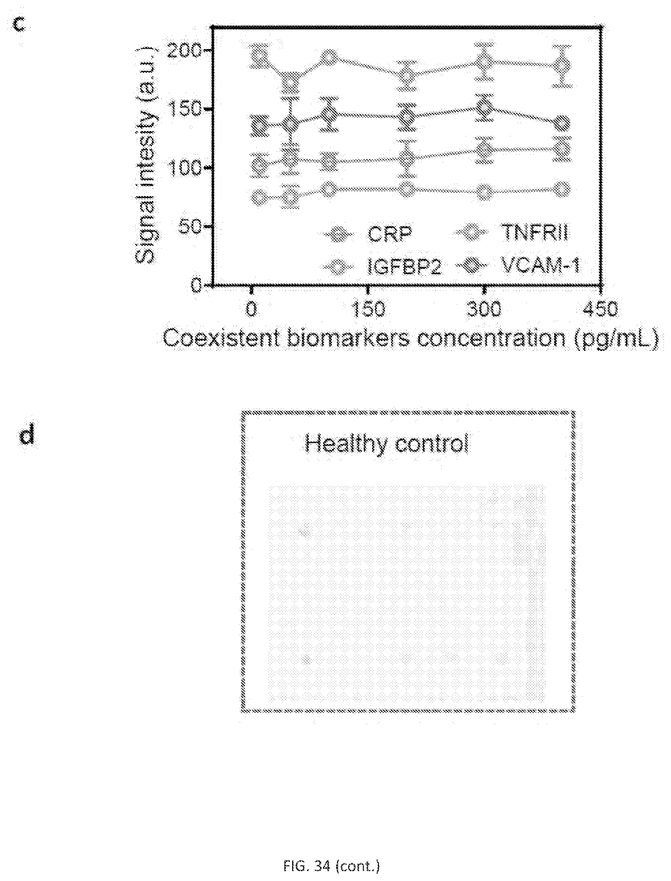

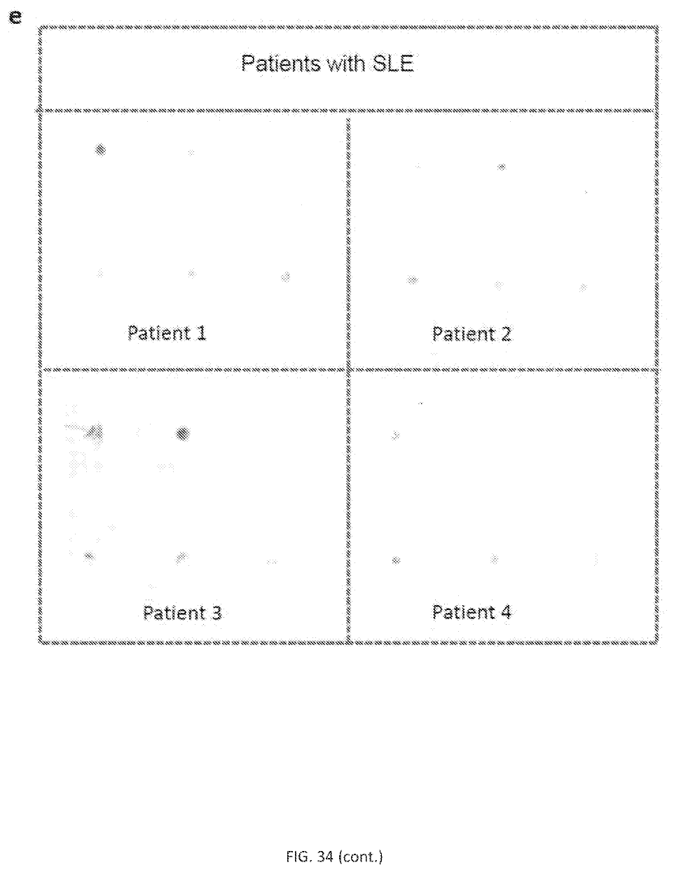

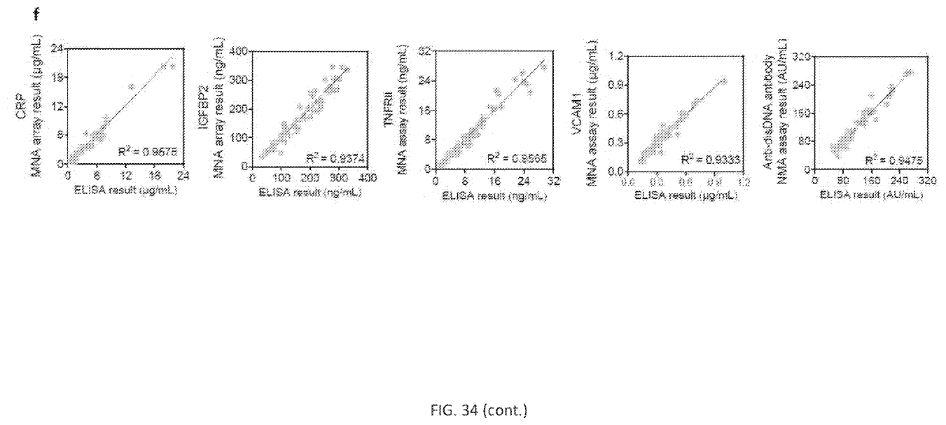

To demonstrate the multiplex biomarker detection ability of MNA, we fabricated an MNA by simultaneous immobilization of CRP, IGFBP2, TNF-RII, VCAM-1, and dsDNA and mBSA, and the location of biomarkers are presented in FIG. 33b. Cross-reactivity is a critical issue in multiplex biomarkers detection assays based on sandwich assay. To evaluate the cross-reactivity of the assay, we prepared a series of solutions containing one targeted biomarker at a fixed concentration while the other four biomarkers with increased concentrations and performed assay on MNA for multiplex biomarkers detection. The results are shown in FIG. 41. For all five biomarkers, when the targeted biomarker was fixed at 20 pg/mL, the signal intensity remained stable, and no significant signal intensity changes were observed with the increased concentration of other four co-existed biomarkers, demonstrating ignorable cross-reactivity of MNA-inked gel assay for multiplex biomarkers detection.

For further demonstration of clinical potential of MNA-inked gel assays for multiplex biomarkers detection, we measured the serum levels of five biomarkers on the same serum samples collected from patients with lupus (N=26) and healthy controls (N=33). According to the results obtained in previous experiments, when the serum samples were diluted 1000 times, all the measurement capable of falling into the linear range of the standard curve of IGFBP2, TNF-RII, and VCAM-1, as established in FIGS. 32a-32d. However, even when we diluted the patient serum by 1,000 times, the concentration of CRP in serum of patients and healthy controls were still much higher than that of the other four biomarkers, thus we optimized the concentration of capture antibody and detection antibody. FIG. 33b displays the representative gel images of healthy controls and SLE patients, and result is presented in Table 6. It is notable that a clear background was observed in all samples, which reveals the assay's high specificity with ignorable influence of other molecules in serum. Similar to the result of MNA-inked gel assays for signal biomarker detection, FIG. 33c exhibits that the paired correlation analysis of the results between ELISA and the MNAs for multiplex biomarker detection showed strong corrections with R² values of 0.9575, 0.9374, 0.9565, 0.9333, and 0.9475 for CRP, IGFBP2, TNF-RII, VCAM-1, and anti-dsDNA antibody, respectively. It is clear that the MNA-inked gel assay is able to provide results comparable to gold-standard test-ELISA reliably and accurately in multiplex biomarker detection. We also compared the MNA results for single biomarker detection and multiplex biomarker detection, and the correlation between them was also strong, with R² values of 0.9772, 0.9760, 0.9730, 0.9262, and 0.9675 for CRP, IGFBP2, TNF-RII, VCAM-1, and anti-dsDNA antibody, respectively, demonstrating that the MNA-inked gel assay has excellent stability of measurement between multi-biomarkers detection and its corresponding single biomarker detection.

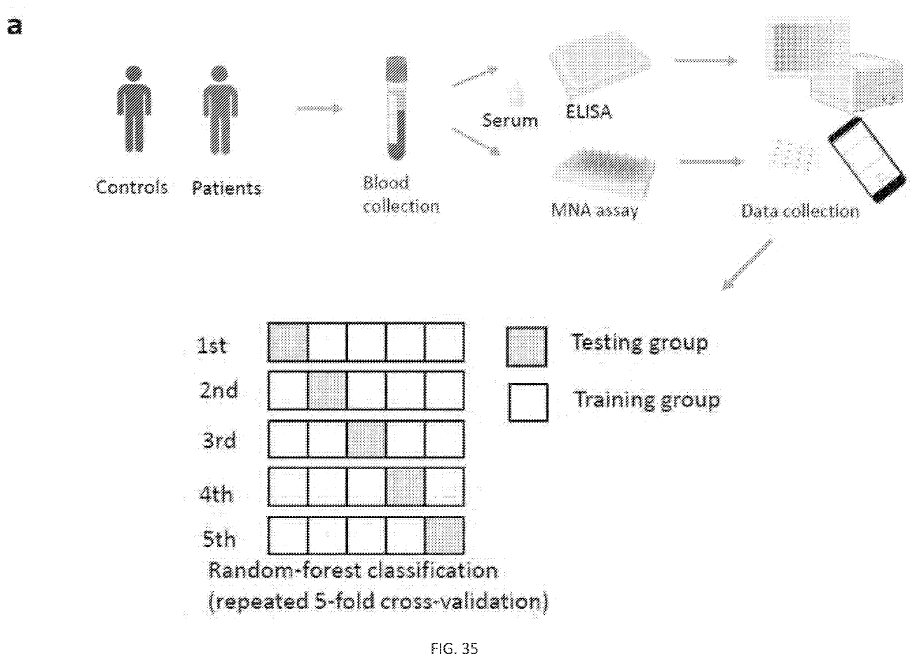

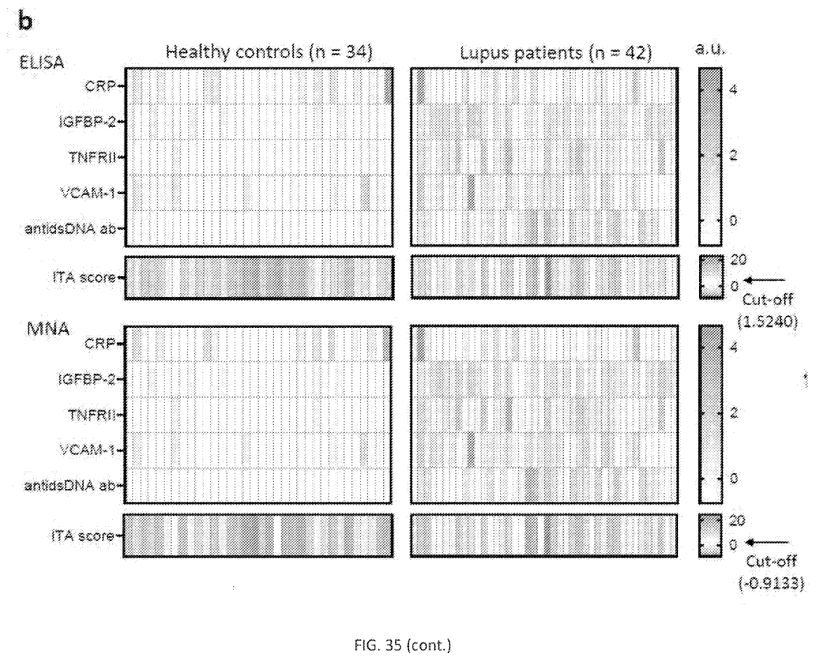

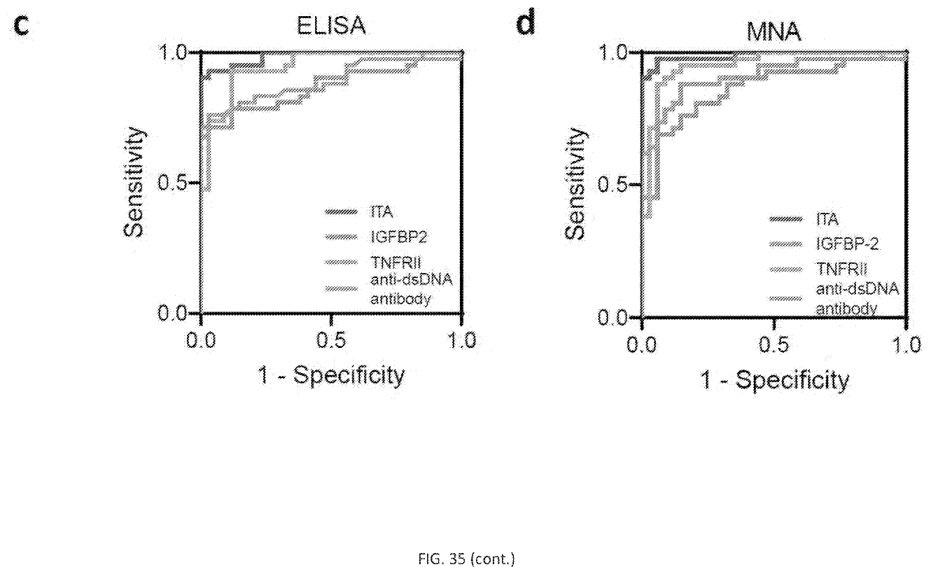

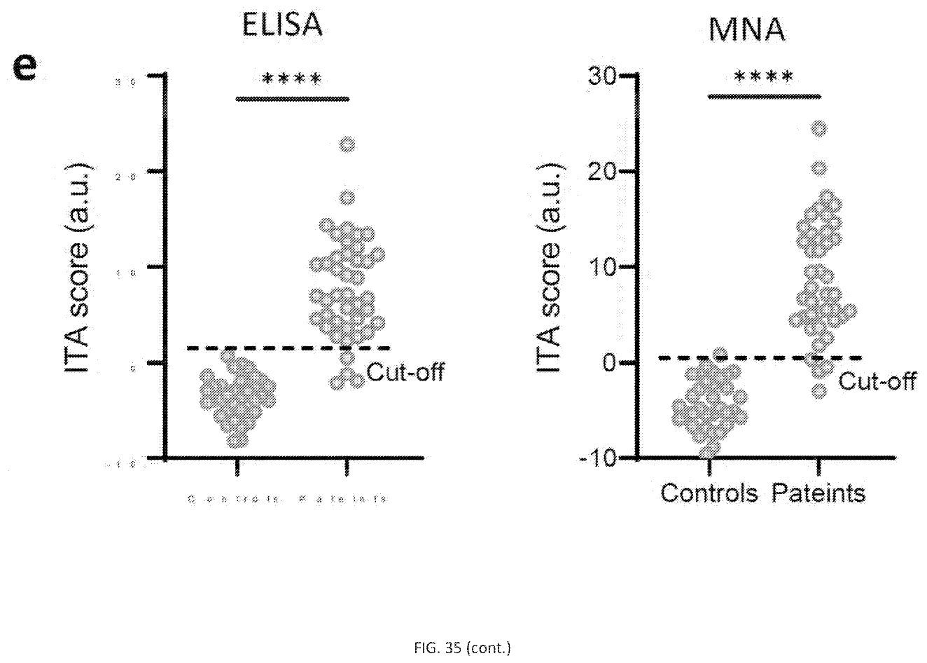

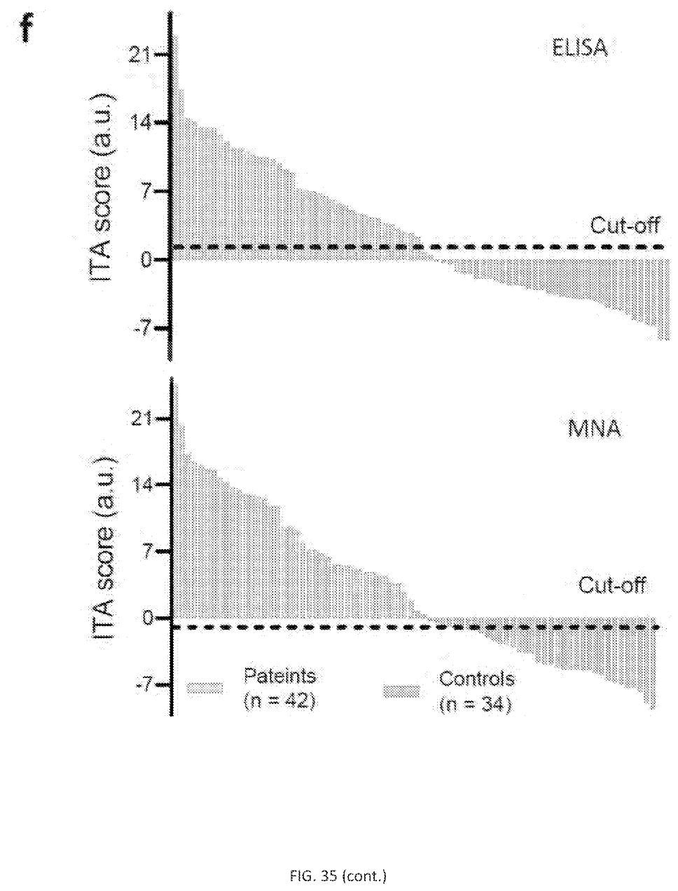

Moreover, we developed a logistic regression model to determine the ability of the selected biomarkers to distinguish SLE patients from healthy controls using levels of biomarker either single or combinations of up to five biomarkers measured by ELISA and MNA gel assay. A five-fold cross-validation method was implemented to prevent overfitting and ensure generalizability (FIG. 35a). To evaluate the performance of the biomarker combinations, weighted sums of selected biomarkers were employed according to the corresponding logistic regression model. Next, receiver operating characteristic (ROC) curves were also generated. Table 4 summarizes the SLE diagnostic performance metrics of biomarkers detected by ELISA and MNA gel assay, including area under the curve (AUC), accuracy, sensitivity, and specificity, which were reported as the average of all 5-folds during cross-validation for both training and testing sets. A final logistic regression model was established using all the data to generate ROC curves, as well as to calculate the AUC. The optimal cutoff values were determined by maximizing the sum of sensitivity and specificity obtained from ROC curves. The results showed that in both assays, the AUC values of IGFBP2, TNFRII, and anti-dsDNA antibodies were greater than 0.8, yet the AUC value of CRP was relatively low (Table 4), which was in agreement with the finding in FIG. 33e. Of note, a biomarker panel with a combination of IGFBP2, TNFRII, and anti-dsDNA antibodies exhibited the best performance, which was defined as an SLE diagnostic index—ITA. Compared with the single biomarker, ITA had a higher AUC value (FIG. 35c,d). Among the testing sets of all the biomarkers and combinations, ITA showed the highest AUC value. It is clear that all AUC values, accuracy, sensitivity, and specificity of biomarkers and combinations obtained from MNA gel assay were close to those of ELISA, further indicating that the results of the MNA gel assay are significantly correlated to those of ELISA. AUC values of ITA determined by MNA gel assay and ELISA in the testing set were 0.9832 and 0.9800, respectively. The ITA scores with a cut-off value of 1.5240 and −0.9133 for ELISA and MNA gel assay between SLE patients and healthy controls had a significant difference, and only a few cases overlayed, demonstrating that the ITA can be used to distinguish SLE patients from healthy controls effectively, and the performance of MNA gel assay for multi-biomarker detection is competitive to ELISA kits (FIG. 35e,f).

Importantly, the specificity of ITA for both ELISA and MNA gel assay in the testing set was found to be 97.14%, representing a 70% improvement in comparison with that of the ANA test, while the sensitivity of ITA was comparable with that of the ANA test. In contrast, the specificity of ANA test is only 57%, which means a higher probability of obtaining false positive results. The low specificity and high sensitivity of ANA test might lead to overdiagnosis and unnecessary treatments. Therefore, the measurement of ITA with high sensitivity and specificity can remarkably improve the diagnostic ability, compared with the ANA test.

Built upon our previous success in the MNA-based sampling of blood biomarkers via laser-pretreated skin, we continuously developed functionalized MNAs aimed at the measurement of a panel of biomarkers, rather than a single biomarker, for onsite diagnosis and monitoring in this study. These functionalized MNAs generated a new platform that can be customized and applied to a variety of analytes or biomarkers for diagnosis, monitoring, and prognosis of various diseases.

The major modification of the MNA surface was decoration of dendrimers that possesses a myriad of primary amine groups on the surface of each microneedle, with which the activated conjugation sites on each microneedle for capture antibody were dramatically increased. The interfacial layer construed by PEI and dendrimer with surface corrugated profile, flexibility of highly branched architecture, and distribution of available functionalized groups for subsequent molecule binding. The modification increased the detection sensitivity by an average of 14.22-fold for five selected biomarkers, compared to ELISA kits. The capture antibody was subsequently linked to the branches of the dendrimer via a novel approach so that a specific antibody could be covalently linked to a target microneedle in the MNA in one antibody-on-one microneedle fashion. A PDMS MNA female mold was slightly modified by replacing the microneedle shape with a cylinder shape by 3D printing. Each of the cylinders functions as a reaction micro-container and can be filled by a specific antibody solution. This approach not only makes it possible to mount many different capture elements in a single MNA in a one-antibody-on-one-microneedle fashion, but also effectively minimize non-specific background signals on the MNA base, which is highly significant for enhancing the specificity and accuracy of the assay. These innovations are critical for multiplex detection of biomarkers, in contrast to the MNA-based assays under development or clinical or preclinical studies that can detect only a single biomarker on one MNA.

After conjugation of specific antibodies in individual microneedles in the MNA, the MNA could be processed similarly as traditional ELISA but with 50× less reagent and sample solutions. Imaging capture and analysis by using MNA has been a major barrier for PoC application, which is larger attributed to difficulty to achieve the same perspective of directly stained microneedles in one image due to the space perspective of microneedles arrangement and 3D structure. Thus, it requires sophisticated equipment for direct imaging. Microneedle not only increase the surface area for immobilization of capture element, but also can transfer color signal with minimization of gel damage. Therefore, “MNA-inked gel assay” is developed, which is based on embedding the colorimetric amplifier in a gel. When the MNA bearing HRP-immunoassay sandwich formation is inserted into the substrate-saturated gel, the signal on a 3D microneedle is converted into a 2D gel for much more convenient and efficient acquisition of signals by smartphone. This MNA-inked gel assay can accommodate as many biomarkers as needed, offering more consistent analysis of the binding signals on each microneedle in the MNA.

The major challenge of multi-biomarker detection is to ensure all the measurement of the samples should be fall in the linear range of biomarkers, after sample dilution. The MNA-inked gel assay that we have developed, has a wide range linear range for CRP detection with low LoD, compared to traditional ELISA kit. Importantly, our assays for single biomarker detection can detect all the samples by just one dilution due to the assays' broader linear ranges with lower LoDs, while CRP ELISA kit required different dilutions to measure different samples due to the broad range of CRP concentration in serum. For multiplex biomarker detection, it is necessary to take CRP and other biomarkers levels into consideration after serum dilution. After estimation, we had to compromise the performance of CRP MNA assay, yet there are still a few samples are out of linear range of the assy. Interestingly, the result of CRP showed no significant difference between SLE patients and healthy controls, thus, in the future application, we can remove CRP from our selected biomarker panel.

There are three innovations in this new platform: First, we are the first to conjugate a specific antibody in a single microneedle in the array, enabling many antibodies to be conjugated on an MNA in a one-antibody-on-one microneedle fashion; Second, greatly amplifying the binding signals by dendrimer modification of the surface of each microneedle; and third, the MNA-inked gel assay allows conversion of the signal on a 3D microneedle into a 2D gel for much more convenient and efficient acquisition of signals by smartphone.

Systemic lupus erythematosus (SLE), a chronic and inflammatory autoimmune disease presenting various abnormalities in a wide range of organs for different patients, might eventually lead to tissue and organ damage caused by the long-term attack of patient's own immune system if the disease activities are not well controlled. Strong association is found between organ damage and elevated risk of SLE mortality, especially cardiovascular and renal damage. For instance, lupus nephritis is the leading cause of death of SLE. SLE predominantly effects women, and has been recognized as the 6^{th} leading cause of death in young women who are at the age between 25 to 34 years in the United States. Therefore, continuous disease activities monitoring during the treatment which can guide further effective therapy and disease management, is critical in minimizing the risk of organ damage and reduce the mortality. However, SLE involves complicated pathological processes, it cannot be determined by merely one biomarker, and a panel of biomarkers measurement should be taken into consideration. Traditionally, detection of biomarkers panel relies on sophisticated and expensive instruments in laboratory settings and is conducted by skilled medical staff, especially for some biomarkers at a low abundance in blood or in some body fluids due to a low detection limit of the instruments. Moreover, long-term and frequent disease activities monitoring increase economic burden of public healthcare and patient. Thus, lupus monitoring at home is highly demanded. Systemic lupus erythematosus (SLE), a severe autoimmune disorder with life-threatening risks, involves complicated pathological processes and can occur fetal complications, being recognized as the leading cause of death in young women.

Collectively, the MNA-inked gel assay developed herein provide an innovated platform for accurate and highly sensitive measurement of a panel of biomarker in a small blood sample volume. We successfully immobilized five capture antibodies in a single MNA in a one-antibody-on-one-microneedle fashion and obtained results with minimum non-specific background signals. The results also show that the assay has higher sensitivity, and specificity with a broader linear range, compared to ELISA kits. We selected a biomarker panel containing five biomarkers in an attempt to distinguish SLE patients from healthy controls. The study measured serum levels of the five biomarkers in SEL patients and healthy controls by using MNA-inked assay for both single and multi-biomarker detection and clinical ELISA kits run side-by-side, so as to provide a proof of concept of our novel MNA-inked assay. The assay and panel biomarkers exhibited discriminative capability in distinguishing SLE patient from healthy control, indicating its future PoC application at home diagnosis and monitoring of SLE, but prospective studies should be further carried to validate the results. Besides, since this assay allows for simple, precise, and efficient image acquisition and analysis of this assay, which can be easily translated to a clinical PoC setting. Finally, the flexibility of the assay enables customization and application for a variety of analytes or biomarkers for diagnosis, monitoring, and prognosis of various diseases in addition to SLE.

Thus, while the invention has been described above in connection with particular embodiments and examples, the invention is not necessarily so limited, and that numerous other embodiments, examples, uses, modifications and departures from the embodiments, examples and uses are intended to be encompassed by the claims attached hereto.

1. A system for identifying a plurality of biomarkers in a sample, comprising:
a substrate comprising a plurality of microneedles projecting therefrom,
each of the plurality of microneedles comprising a plurality of biomarker recognition molecules attached thereto, and
the plurality of microneedles including a first microneedle and a second microneedle,
the first microneedle comprising a first plurality of biomarker recognition molecules configured to recognize a first biomarker, and
the second microneedle comprising a second plurality of biomarker recognition molecules configured to recognize a second biomarker different from the first biomarker.

2. The system of claim 1, wherein each of the plurality of biomarker recognition molecules is coupled to a respective microneedle of the plurality of microneedles using a plurality of dendritic linking molecules,
wherein each of the plurality of dendritic linking molecules couples multiple biomarker recognition molecules to the respective microneedle.

3. The system of claim 2, wherein the plurality of biomarker recognition molecules comprise at least one of antibodies, aptamers, or ligands.

4. The system of claim 3, wherein, upon exposure to a sample comprising a plurality of sample biomarkers, each of the plurality of biomarker recognition molecules associated with each of the respective plurality of microneedles is configured to recognize and couple to a respective biomarker of the plurality of sample biomarkers.

5. The system of claim 4, wherein, after exposure to the sample, each of the plurality of microneedles is processed to include a labeling compound to identify each of the plurality of sample biomarkers.

6. The system of claim 5, further comprising a gel overlay contacting the plurality of microneedles,
wherein the gel overlay comprises a labeling substrate configured to form a precipitate within the gel overlay when contacted by the labeling compound.

7. (canceled)

8. The system of claim 6, wherein the labeling compound comprises horseradish peroxidase (HRP), and
wherein the labeling substrate comprises an HRP substrate.

9. The system of claim 1, further comprising an imaging adapter configured to collect an image from at least one of the plurality of microneedles or a gel overlay that has contacted the plurality of microneedles.

10. The system of claim 9, wherein the imaging adapter comprises a phone holder configured to align a camera of a phone with an imaging system,
wherein the imaging system comprises at least one lens, a light source, and a specimen holder, and
wherein the specimen holder is configured to hold at least one of the gel overlay or the plurality of microneedles.

11-12. (canceled)

13. The system of claim 10, wherein the specimen holder comprises the gel overlay, and
wherein the camera of the phone is configured to obtain an image of the gel overlay using the imaging system.

14. The system of claim 10, wherein the specimen holder comprises a mechanical stage configured to adjust a position of the at least one of the gel overlay or the plurality of microneedles.

15. The system of claim 14, wherein the specimen holder comprises the plurality of microneedles,
wherein the at least one lens comprises a micro lens and a magnifying lens configured to obtain an enlarged image of a microneedle of the plurality of microneedles, and
wherein the mechanical stage is configured to adjust the position of the plurality of microneedles in three dimensions such that the camera of the phone obtains an enlarged image of each of the plurality of microneedles.

16-18. (canceled)

19. The system of claim 10, wherein the phone holder further comprises a disk centrifuge configured to process the sample using centrifugal force to separate a test portion of the sample from a remaining portion of the sample,
wherein the disk centrifuge comprises a circular disk including a sample holder attached thereto in a radial configuration.

20-21. (canceled)

22. The system of claim 1, further comprising a biomarker recognition molecule preparation chamber including a plurality of microwells,
wherein each of the plurality of microwells is configured to accommodate a microneedle of the plurality of microneedles to separately attach each of the plurality of biomarker recognition molecules to each of the plurality of microneedles.

23. The system of claim 22, wherein the biomarker recognition molecule preparation chamber comprises an overlay,
wherein the plurality of microwells comprise a plurality of openings extending through the overlay, and
wherein the overlay is placed over the plurality of microneedles and attached to the substrate such that each of the plurality of openings provides a microcontainer for attaching a biomarker recognition molecules to a microneedle.

24. The system of claim 1, further comprising an immunoassay station comprising a microfluidic array configured to prepare the sample,
wherein the microfluidic array comprises at least one of a sample dilution fluid, a washing buffer, a detection antibody mixture, or a substrate solution disposed therein.

25. (canceled)

26. The system of claim 24, wherein the immunoassay station further comprises a manual vacuum system configured to draw at least one of the sample dilution fluid, the washing buffer, the detection antibody mixture, or the substrate solution from the microfluidic array to contact the plurality of microneedles.

27. The system of claim 1, wherein each of the plurality of microneedles comprises a tapered end,
wherein each of the plurality of microneedles comprises an optically transparent material, and
wherein the tapered end of each of the plurality of microneedles is configured to penetrate at least one of an epidermis or a dermis of a subject.

28. The system of claim 27, further comprising a light source configured to deliver light to the subject using each of the plurality of microneedles,
wherein the light source comprises a laser including a microlens array configured to create a plurality of beams to deliver light to the subject using each of the plurality of microneedles.

29. (canceled)

30. The system of claim 28, wherein each of the plurality of beams is transmitted to each of the plurality of microneedles using a respective plurality of fiber bundles,
wherein each of the plurality of fiber bundles comprises a central fiber for delivering excitation light surrounded by a plurality of peripheral fibers for collecting emission light.

31-63. (canceled)
